(19) United States
(12) Patent Application Publication
(10) Pub. No.: US 2024/0209379 A1
(43) Pub. Date: Jun. 27, 2024

(54) COMPOSITIONS AND METHODS FOR ENHANCING GENE EXPRESSION

(71) Applicant: Janssen Pharmaceuticals, Inc., Titusville, NJ (US)

(72) Inventors: Kurt Iver KAMRUD, San Diego, CA (US); Maung Nyan WIN, San Diego, CA (US); Nathaniel Stephen WANG, San Diego, CA (US); Jason L. DEHART, San Diego, CA (US)

(21) Appl. No.: 18/498,448

(22) Filed: Oct. 31, 2023

Related U.S. Application Data
(62) Division of application No. 15/831,230, filed on Dec. 4, 2017, now Pat. No. 11,845,939.
(60) Provisional application No. 62/587,954, filed on Nov. 17, 2017.
(60) Provisional application No. 62/486,361, filed on Apr. 17, 2017.
(60) Provisional application No. 62/430,250, filed on Dec. 5, 2016.

(51) Int. Cl. C12N 15/63; A61K 48/00; C12N 15/113; C12N 15/85; C12N 15/86

(52) U.S. Cl. CPC C12N 15/63; A61K 48/005; C12N 15/113; C12N 15/86; C07K 2319/50; C12N 2015/8518; C12N 2310/531; C12N 2770/00042; C12N 2770/36062; C12N 2770/36111; C12N 2770/36143; C12N 2830/30; C12N 2840/105

(57) ABSTRACT

The present disclosure generally relates to nucleic acid molecules for use in regulating gene expression. Disclosed herein include nucleic acid molecules containing one or more structural elements of the viral capsid enhancer operably linked to a coding sequence of a gene of interest. In some embodiments, the viral capsid enhancer comprises a Downstream Loop (DLP) from a viral capsid protein, or a variant of the DLP.

### RELATED APPLICATIONS

The present application claims priority to U.S. Provisional Application Ser. No. 62/430,250, filed on Dec. 5, 2016; U.S. Provisional Application Ser. No. 62/486,361, filed on Apr. 17, 2017; and U.S. Provisional Application Ser. No. 62/587,954, filed on Nov. 17, 2017. The contents of the above-referenced applications are hereby expressly incorporated by reference in their entireties.

### INCORPORATION OF THE SEQUENCE LISTING

The material in the accompanying sequence listing is hereby incorporated by reference into this application. The accompanying sequence listing text file, named SGI012A_SeqListing.txt, was created on Dec. 4, 2017 and is 169 KB.

### FIELD

The present disclosure relates to the field of molecular biology and genetic engineering, including nucleic acid molecules useful for regulating gene expression, and the use of the nucleic acid molecules for, for example, production of desired products in suitable host cells in cell culture or in a subject, and for conferring beneficial characteristics to the host cells or subjects.

### BACKGROUND

Advances in biotechnology and molecular biology have offered many opportunities to develop recombinant cells and organisms with commercially desirable characteristics or traits. In particular, modern genetic engineering techniques have greatly accelerated the introduction of genes and hence new traits into recombinant cells and organisms. Proper expression level of a desirable gene in, for example, a host cell or a transgenic organism is helpful to achieve this goal.

However, despite the availability of many molecular tools, genetic modifications of host cells and organisms are often constrained by insufficient expression level or uncontrolled expression of the gene of interest. Thus, there is still a need for regulatory elements capable of enhancing transgene expression in host cells and organisms. The identification of novel molecular tools including regulatory elements, expression vector, and expression systems that function in various types of organisms can be useful in developing genetically enhanced cells and organisms.

### SUMMARY

This section provides a general summary of the present application, and is not comprehensive of its full scope or all of its features.

The present disclosure relates generally to methods and compositions useful for regulating, for example increasing, gene expression in vitro, ex vivo, or in vivo. The gene expression can be, for example, in animal cells and other eukaryotic cells. The gene can be, for example, a heterologous gene encoding a protein of interest.

In one aspect, some embodiments disclosed herein relate to a nucleic acid molecule, including (i) a first nucleic acid sequence encoding one or more RNA stem-loops of a viral capsid enhancer or a variant thereof; and (ii) a second nucleic acid sequence operably linked to the first nucleic acid sequence, wherein the second nucleic acid sequence comprises a coding sequence for a gene of interest (GOI).

Implementations of embodiments of the nucleic acid molecule according to the present disclosure can include one or more of the following features. In some embodiments, the first nucleic acid sequence is operably linked upstream to the coding sequence for the GOI. In some embodiments, the nucleic acid molecule further includes a promoter operably linked upstream to the first nucleic acid sequence. In some embodiments, the nucleic acid molecule further includes a 5′ UTR sequence operably linked upstream to the first nucleic acid sequence. In some embodiments, the 5′ UTR sequence is operably linked downstream to the promoter and upstream to the first nucleic acid sequence. In some embodiments, the nucleic acid molecule further includes a coding sequence for an autoprotease peptide operably linked upstream to the second nucleic acid sequence. In some embodiments, the coding sequence for the autoprotease peptide is operably linked downstream to the first nucleic acid sequence and upstream to the second nucleic acid sequence. In some embodiments, the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the nucleic acid molecule further includes a 3′ UTR sequence operably linked downstream to the second sequence nucleic acid sequence.

In some embodiments, the viral capsid enhancer is derived from a capsid gene of a virus species belonging to the Togaviridae family. In some embodiments, the virus species belongs to the Alphavirus genus of the Togaviridae family. In some embodiments, the alphavirus species is Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), Salmonid alphavirus (SAV), or Buggy Creek virus. In some embodiments, the viral capsid enhancer comprises a downstream loop (DLP) motif of the virus species, and wherein the DLP motif comprises at least one of the one or more RNA stem-loops. In some embodiments, the viral capsid enhancer comprises a nucleic acid sequence exhibiting at least 80% sequence identity to at least one of SEQ ID NOs: 1 and 46-52. In some embodiments, the nucleic acid sequence exhibits at least 95% sequence identity to at least one of SEQ ID NOs: 1 and 46-52.

In some embodiments, the coding sequence for the GOI encodes a polypeptide. In some embodiments, the polypeptide is a therapeutic polypeptide, a prophylactic polypeptide, a diagnostic polypeptide, a nutraceutical polypeptide, an industrial enzyme, a reporter polypeptide, or a combination thereof. In some embodiments, the polypeptide is an antibody, an antigen, an immune modulator, a cytokine, an enzyme, or a combination thereof.

In some embodiments, the nucleic acid molecule of the disclosure further includes a third nucleic acid sequence encoding one or more RNA stem-loops of a second viral capsid enhancer or a variant thereof; and a fourth nucleic acid sequence operably linked to the third nucleic acid sequence, wherein the fourth nucleic acid sequence comprises a coding sequence for a second gene of interest (GOI). In some embodiments, the nucleic acid molecule further includes a coding sequence for a second autoprotease peptide operably linked downstream to the third nucleic acid sequence and upstream to the fourth nucleic acid sequence.

In some embodiments, the nucleic acid molecule of the disclosure is an mRNA molecule or an RNA replicon. In some embodiments, the nucleic acid molecule is an expression vector or a transcription vector. In some embodiments, the expression vector or a transcription vector further includes one or more additional transcription regulatory sequences. In some embodiments, the expression vector or a transcription vector further includes one or more additional transcription regulatory sequences. In some embodiments, the expression vector or a transcription vector further includes one or more additional translation regulatory sequences. In some embodiments, the nucleic acid molecule is a plasmid, a bacteriophage vector, a cosmid, a fosmid, a viral replicon, a shuttle vector, or a combination thereof. In some embodiments, the nucleic acid molecule is a prokaryotic vector or a eukaryotic vector. In some embodiments, the nucleic acid molecule is produced via de novo synthesis.

Also disclosed in some embodiments include a method for producing a polypeptide of interest in a cell, which includes introducing a nucleic acid molecule of according to the present disclosure into a cell, thereby producing a polypeptide encoded by the GOI in the cell. In yet another related aspect, some embodiments disclosed herein related to a method for producing a polypeptide of interest in a cell, which includes introducing a RNA molecule into the cell, wherein the RNA molecule comprises one or more RNA stem-loops of a viral capsid enhancer or a variant thereof, and a coding sequence for the polypeptide of interest, thereby producing the polypeptide of interest in the cell.

In some embodiments, the RNA molecule is a messenger RNA (mRNA) molecule or a replicon RNA molecule. In some embodiments, the RNA molecule is produced via de novo synthesis and/or in vitro transcription before being introduced into the cell. In some embodiments, the RNA molecule comprises a downstream loop (DLP) motif of a virus species, and wherein the DLP motif comprises at least one of the one or more RNA stem-loops of the viral capsid enhancer. In some embodiments, the RNA molecule further comprises a coding sequence for an autoprotease peptide downstream to at least one of the one or more RNA stem-loops and upstream to the coding sequence for the polypeptide of interest. In some embodiments, the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the polypeptide is a therapeutic polypeptide, a prophylactic polypeptide, a diagnostic polypeptide, a nutraceutical polypeptide, an industrial enzyme, a reporter polypeptide, or a combination thereof. In some embodiments, the polypeptide is an antibody, an antigen, an immune modulator, a cytokine, an enzyme, or a combination thereof. In some embodiments, the cell is present in a tissue, an organ, or a subject. In some embodiments, the subject is human, horse, pig, primate, mouse, ferret, rat, cotton rat, cattle, swine, sheep, rabbit, cat, dog, bird, fish, goat, donkey, hamster, or buffalo.

Some embodiments disclose a method for producing a messenger RNA (mRNA) in a cell. The method, in some embodiments, includes administering to the cell a nucleic acid molecule comprising a first nucleic acid sequence encoding one or more RNA stem-loops of a viral capsid enhancer or a variant thereof, and a second nucleic acid sequence operably linked to the first nucleic acid sequence, wherein the second nucleic acid sequence comprises a coding sequence for a gene of interest (GOI), thereby producing a mRNA of the GOI.

In some embodiments, the first nucleic acid sequence is operably linked upstream to the coding sequence for the GOI. In some embodiments, the nucleic acid molecule further includes a promoter operably linked upstream to the first nucleic acid sequence. In some embodiments, the nucleic acid molecule further includes a 5′ UTR sequence operably linked upstream to the first nucleic acid sequence. In some embodiments, the 5′ UTR sequence is operably linked downstream to the promoter and upstream to the first nucleic acid sequence. In some embodiments, the nucleic acid molecule further includes a coding sequence for an autoprotease peptide operably linked upstream to the second nucleic acid sequence. In some embodiments, the coding sequence for the autoprotease peptide is operably linked downstream to the first nucleic acid sequence and upstream to the second nucleic acid sequence. In some embodiments, the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the nucleic acid molecule further includes a 3′ UTR sequence operably linked downstream to the second sequence nucleic acid sequence.

In some embodiments disclosed herein, the viral capsid enhancer is derived from a capsid gene of a virus species belonging to the Togaviridae family. In some embodiments, the virus species belongs to the Alphavirus genus of the Togaviridae family. In some embodiments, the alphavirus species is Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), Salmonid alphavirus (SAV), or Buggy Creek virus. In some embodiments, the viral capsid enhancer comprises a downstream loop (DLP) motif of the virus species, and wherein the DLP motif comprises at least one of the one or more RNA stem-loops. In some embodiments, the viral capsid enhancer comprises a nucleic acid sequence exhibiting at least 80% sequence identity to at least one of SEQ ID NOs: 1 and 46-52. In some embodiments, the nucleic acid sequence exhibits at least 95% sequence identity to at least one of SEQ ID NOs: 1 and 46-52.

In some embodiments disclosed herein, the coding sequence for the GOI encodes a polypeptide. In some embodiments, the polypeptide is selected from the group consisting of a therapeutic polypeptide, a prophylactic polypeptide, a diagnostic polypeptide, a nutraceutical polypeptide, an industrial enzyme, a reporter polypeptide, and a combination thereof. In some embodiments, the polypeptide is an antibody, an antigen, an immune modulator, a cytokine, an enzyme, or a combination thereof. In some embodiments of the method for producing a messenger RNA (mRNA) according to the present disclosure, the nucleic acid molecule further includes a third nucleic acid sequence encoding one or more RNA stem-loops of a second viral capsid enhancer or a variant thereof; and a fourth nucleic acid sequence operably linked to the third nucleic acid sequence, wherein the fourth nucleic acid sequence comprises a coding sequence for a second gene of interest (GOI). In some embodiments, the nucleic acid molecule further includes a coding sequence for a second autoprotease peptide operably linked downstream to the third nucleic acid sequence and upstream to the fourth nucleic acid sequence.

The nucleic acid molecule of the present disclosure can be, in some embodiments, an RNA replicon. In some embodiments, the nucleic acid molecule is an expression vector or a transcription vector. In some embodiments, the nucleic acid molecule further comprises one or more additional transcription regulatory sequences. In some embodiments, the nucleic acid molecule further comprises. In some embodiments, one or more additional translation regulatory sequences. In some embodiments, the nucleic acid molecule is an expression vector selected from the group consisting of a plasmid, a bacteriophage vector, a cosmid, a fosmid, a viral replicon, a shuttle vector, and a combination thereof. In some embodiments, the nucleic acid molecule is a prokaryotic expression vector or a eukaryotic expression vector. In some embodiments, the cell is present in a tissue, an organ, or a subject. In some embodiments, the subject is human, horse, pig, primate, mouse, ferret, rat, cotton rat, cattle, swine, sheep, rabbit, cat, dog, bird, fish, goat, donkey, hamster, or buffalo. In some embodiments of the method for producing a messenger RNA (mRNA) according to the present disclosure further includes producing a polypeptide encoded by the mRNA of the GOI in the cell. In some embodiments, the method further includes obtaining the produced mRNA of the GOI and introducing the obtained mRNA into a second cell for expression of a polypeptide encoded by the mRNA of the GOI in the second cell.

In one aspect, some embodiments of the disclosure relate to nucleic acid molecule comprising a nucleic acid sequence encoding a modified viral RNA replicon, wherein the modified viral RNA replicon comprises (i) a first nucleic acid sequence encoding one or more structural elements of a viral capsid enhancer or a variant thereof, wherein the viral capsid enhancer is heterologous to the viral RNA replicon, and (ii) a second nucleic acid sequence encoding at least one nonstructural viral protein or a portion thereof, wherein the first nucleic acid sequence is operably linked upstream to the second nucleic acid sequence.

In some embodiments, at least one of the one or more structural elements of the viral capsid enhancer comprises one or more RNA stem-loops. In some embodiments, the viral capsid enhancer is derived from a capsid gene of a virus species belonging to the Togaviridae family. In some embodiments, the virus species belongs to the Alphavirus genus of the Togaviridae family. In some embodiments, the alphavirus species Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), or Buggy Creek virus. In some embodiments, the viral capsid enhancer comprises a downstream loop (DLP) motif of the virus species, and wherein the DLP motif comprises at least one of the one or more RNA stem-loops. In some embodiments, the viral capsid enhancer comprises a nucleic acid sequence exhibiting at least 80% sequence identity to at least one of SEQ ID NOs: 1 and 46-52. In some embodiments, the nucleic acid sequence exhibits at least 95% sequence identity to at least one of SEQ ID NOs: 1 and 46-52.

In some embodiments, the nucleic acid sequence encoding the modified viral RNA replicon further comprising a coding sequence for an autoprotease peptide operably linked downstream to the first nucleic acid sequence and upstream to the second nucleic acid sequence. In some embodiments, the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof. In some embodiments, the first nucleic acid sequence is operably positioned within a region of about 1 to 1000 nucleotides downstream of the 5′-terminus of the modified viral RNA replicon. the second nucleic acid sequence comprises substantially all the coding sequence for the native viral nonstructural proteins of the corresponding unmodified viral RNA replicon.

In some embodiments disclosed herein, the modified viral RNA replicon comprises a modified RNA replicon derived from a virus species belonging to the Alphavirus genus of the Togaviridae family or to the Arterivirus genus of the Arteriviridae family.

In some embodiments, the arterivirus virus species is Equine arteritis virus (EAV), Porcine respiratory and reproductive syndrome virus (PRRSV), Lactate dehydrogenase elevating virus (LDV), or Simian hemorrhagic fever virus (SHFV). In some embodiments, the first nucleic acid sequence is operably positioned upstream to a second nucleic acid sequence encoding a portion or the entire pp1ab nonstructural protein of the modified arterivirus RNA replicon. In some embodiments, the nucleic acid sequence encoding the modified arterivirus RNA replicon further comprising one or more expression cassettes, wherein at least one of the one or more expression cassettes comprises a promoter operably linked to a coding sequence for a gene of interest (GOI). In some embodiments, the modified arterivirus RNA replicon comprises at least two, three, four, five, or six expression cassettes. In some embodiments, at least one of the one or more expression cassettes is operably linked downstream of the second nucleic acid sequence encoding a portion or the entire pp1ab nonstructural protein of the modified arterivirus RNA replicon. In some embodiments, at least one of the one or more expression cassettes is operably positioned downstream to a transcriptional regulatory sequence (TRS) of the modified arterivirus RNA replicon, wherein the TRS is TRS1, TRS2, TRS3, TRS4, TRS5, TRS6, or TRS7. In some embodiments, at least one of the one or more expression cassettes further comprises a third nucleic acid sequence encoding one or more structural elements of a viral capsid enhancer, wherein the third nucleic acid sequence is operably linked upstream to the coding sequence for the GOI.

In some embodiments, the nucleic acid sequence encoding the modified arterivirus RNA replicon further comprises a coding sequence for an autoprotease peptide operably linked downstream to the third nucleic acid sequence and upstream to the coding sequence for the GOI. In some embodiments, the coding sequence for the GOI encodes a therapeutic polypeptide, a prophylactic polypeptide, a diagnostic polypeptide, a nutraceutical polypeptide, an industrial enzyme, a reporter polypeptide, or any combination thereof. In some embodiments, the coding sequence for the GOI encodes an antibody, an antigen, an immune modulator, a cytokine, an enzyme, or any combination thereof.

In some embodiments, the modified viral RNA replicon comprises a modified RNA replicon derived from an alphavirus virus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), Salmonid alphavirus (SAV), and Buggy Creek virus. In some embodiments, the first nucleic acid sequence is operably positioned upstream to a second nucleic acid sequence encoding one or more nonstructural proteins nsp1-4 or a portion thereof of the modified alphavirus RNA replicon. In some embodiments, the nucleic acid sequence encoding the modified alphavirus RNA replicon further comprises one or more expression cassettes, wherein each of the expression cassettes comprises a promoter operably linked to a coding sequence for a gene of interest (GOI). In some embodiments, the modified alphavirus RNA replicon comprises at least two, three, four, five, or six expression cassettes. In some embodiments, t least one of the one or more expression cassettes is operably linked downstream of a nucleic acid sequence encoding one or more nonstructural proteins nsp1-4 or a portion thereof of the modified alphavirus RNA replicon. In some embodiments, at least one of the one or more expression cassettes further comprises a third nucleic acid sequence encoding one or more structural elements of a viral capsid enhancer, wherein the third nucleic acid sequence is operably linked upstream of the coding sequence for the GOI. In some embodiments, the nucleic acid sequence encoding the modified alphavirus RNA replicon further comprises a coding sequence for an autoprotease peptide operably linked downstream to the third nucleic acid sequence and upstream to the coding sequence for the GOI. In some embodiments, the coding sequence for the GOI encodes a therapeutic polypeptide, a prophylactic polypeptide, a diagnostic polypeptide, a nutraceutical polypeptide, an industrial enzyme, a reporter polypeptide, or a combination thereof. In some embodiments, the coding sequence for the GOI encodes an antibody, an antigen, an immune modulator, an enzyme, a cytokine, or a combination thereof.

In one aspect, some embodiments of the disclosure relate to nucleic acid molecule comprising a nucleic acid sequence encoding a modified non-alphavirus RNA replicon, wherein the modified non-alphavirus RNA replicon comprising a first nucleic acid sequence encoding one or more structural elements of a viral capsid enhancer or a variant thereof. In some embodiments, the nucleic acid sequence encoding the modified non-alphavirus RNA replicon further comprises a second nucleic acid sequence encoding at least one nonstructural viral protein or a portion thereof, wherein the first nucleic acid sequence is operably linked upstream to the second nucleic acid sequence. In some embodiments, nucleic acid sequence encoding the modified non-alphavirus RNA replicon further comprises a coding sequence for an autoprotease peptide operably linked downstream to the first nucleic acid sequence and upstream to the second nucleic acid sequence. In some embodiments, the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof. In some embodiments, the nucleic acid sequence encoding the modified non-alphavirus RNA replicon comprises a modified RNA replicon derived from a positive-strand RNA virus. In some embodiments, the positive-strand RNA virus is a virus species belonging to a family selected from the group consisting of Togaviridae family, Flaviviridae family, Orthomyxoviridae family, Rhabdoviridae family, and Paramyxoviridae family. In some embodiments, the positive-strand RNA virus is a virus species belonging to the Arterivirus genus of the Arteriviridae family.

In some embodiments disclosed herein, the nucleic acid sequence encoding the modified non-alphavirus RNA replicon further comprising one or more expression cassettes, wherein each of the expression cassettes comprises a promoter operably linked to a coding sequence for a gene of interest (GOI). In some embodiments, the modified non-alphavirus RNA replicon comprises at least two, three, four, five, or six expression cassettes. In some embodiments, at least one of the one or more expression cassettes is operably linked downstream of the second nucleic acid sequence encoding the at least one nonstructural viral protein or a portion thereof. In some embodiments, at least one of the one or more expression cassettes further comprises a third nucleic acid sequence encoding one or more structural elements of a viral capsid enhancer, wherein the third nucleic acid sequence is operably linked upstream to the coding sequence for the GOI. In some embodiments, the nucleic acid sequence encoding the modified non-alphavirus RNA replicon further comprising a coding sequence for an autoprotease peptide operably linked downstream to the third nucleic acid sequence and upstream to the coding sequence for the GOI. In some embodiments, the nucleic acid molecule is produced via de novo synthesis.

In one aspect, some embodiments disclosed herein relate to a recombinant cell including a nucleic acid molecule as disclosed herein. In some embodiments, the recombinant cell is a prokaryotic cell or a eukaryotic cell. In some embodiments, the recombinant cell is an animal cell. In some embodiments, the nucleic acid molecule comprises a nucleic acid sequence encoding a modified RNA replicon, and wherein expression of the modified replicon RNA confers a resistance to innate immune response in the recombinant cell. In a related aspect, some embodiments disclosed herein relate to a cell culture which includes at least one recombinant cell as disclosed herein.

In some aspects, some embodiments disclosed herein relate to a method for conferring a resistance to the innate immune system in a subject which includes administering to the subject a nucleic acid molecule comprising a nucleic acid sequence which encodes a modified viral RNA replicon, wherein the modified viral RNA replicon comprises (i) a first nucleic acid sequence encoding one or more structural elements of a viral capsid enhancer or a variant thereof, wherein the viral capsid enhancer is heterologous to the viral RNA replicon, and (ii) a second nucleic acid sequence encoding at least one nonstructural protein or a portion thereof, wherein the first nucleic acid sequence is operably linked upstream to the second nucleic acid sequence, and wherein expression of the modified replicon RNA encoded by the nucleic acid molecule confers a resistance to innate immune response in the subject. In some embodiments, the subject is selected from the group consisting of human, horse, pig, primate, mouse, ferret, rat, cotton rat, cattle, swine, sheep, rabbit, cat, dog, bird, fish, goat, donkey, hamster, and buffalo

In some aspect, some embodiments disclosed herein relate to a method for producing a polypeptide of interest in a subject which includes administering to the subject a nucleic acid molecule comprising a nucleic acid sequence which encodes a modified viral RNA replicon, wherein the modified viral RNA replicon comprises (i) a first nucleic acid sequence encoding one or more structural elements of a viral capsid enhancer or a variant thereof, wherein the viral capsid enhancer is heterologous to the viral RNA replicon, and (ii) a second nucleic acid sequence encoding at least one nonstructural protein or a portion thereof, wherein the first nucleic acid sequence is operably linked upstream to the second nucleic acid sequence. In some embodiments, the subject is human, horse, pig, primate, mouse, ferret, rat, cotton rat, cattle, swine, sheep, rabbit, cat, dog, bird, fish, goat, donkey, hamster, or buffalo.

In some aspect, some embodiments disclosed herein relate to a method for producing a polypeptide of interest, which includes ulturing a host cell comprising a nucleic acid molecule which comprises a nucleic acid sequence encoding a modified viral RNA replicon, wherein the modified viral RNA replicon comprises (i) a first nucleic acid sequence encoding one or more structural elements of a viral capsid enhancer or a variant thereof, wherein the viral capsid enhancer is heterologous to the viral RNA replicon, and (ii) a second nucleic acid sequence encoding at least one nonstructural protein or a portion thereof, wherein the first nucleic acid sequence is operably linked upstream to the second nucleic acid sequence.

In some embodiments of the method for producing a polypeptide of interest according to the present disclosure, the subject is selected from the group consisting of human, horse, pig, primate, mouse, ferret, rat, cotton rat, cattle, swine, sheep, rabbit, cat, dog, bird, fish, goat, donkey, hamster, and buffalo. In some embodiments, at least one of the one or more structural elements of the viral capsid enhancer comprises one or more RNA stem-loops. In some embodiments, the viral capsid enhancer is derived from a capsid gene of a virus species belonging to the Togaviridae family. In some embodiments, the virus species belongs to the Alphavirus genus of the Togaviridae family. In some embodiments, the alphavirus species is Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), or Buggy Creek virus. In some embodiments, the viral capsid enhancer comprises a downstream loop (DLP) motif of the virus species, and wherein the DLP motif comprises at least one of the one or more RNA stem-loops. In some embodiments, the viral capsid enhancer comprises a nucleic acid sequence exhibiting at least 80% sequence identity to at least one of SEQ ID NOs: 1 and 46-52. In some embodiments, the nucleic acid sequence exhibits at least 95% sequence identity to at least one of SEQ ID NOs: 1 and 46-52.

In some embodiments disclosed herein, the nucleic acid sequence encoding the modified viral RNA replicon further comprising a coding sequence for an autoprotease peptide operably linked downstream to the first nucleic acid sequence and upstream to the second nucleic acid sequence. In some embodiments, the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof. In some embodiments, the first nucleic acid sequence is operably positioned within a region of about 1 to 1000 nucleotides downstream of the 5′-terminus of the modified viral RNA replicon. the second nucleic acid sequence comprises substantially all the coding sequence for the native viral nonstructural proteins of the corresponding unmodified viral RNA replicon.

In some embodiments, the modified viral RNA replicon comprises a modified RNA replicon derived from a virus species belonging to the Alphavirus genus of the Togaviridae family or to the Arterivirus genus of the Arteriviridae family. In some embodiments, the arterivirus virus species is Equine arteritis virus (EAV), Porcine respiratory and reproductive syndrome virus (PRRSV), Lactate dehydrogenase elevating virus (LDV), or Simian hemorrhagic fever virus (SHFV).

In some embodiments disclosed herein, the nucleic acid sequence encoding the modified arterivirus RNA replicon further comprises one or more expression cassettes, and wherein at least one of the expression cassettes comprises a promoter operably linked to a coding sequence for a gene of interest (GOI). In some embodiments, the virus species is an arterivirus, and wherein the first nucleic acid sequence is operably positioned upstream to a nucleic acid sequence encoding a portion or the entire pp1ab nonstructural protein of the modified arterivirus RNA replicon. In some embodiments, the modified arterivirus RNA replicon further comprises at least two, three, four, five, or six expression cassettes. In some embodiments, at least one of the one or more expression cassettes is operably linked downstream of the second nucleic acid sequence encoding a portion or the entire pp1ab nonstructural protein of the modified arterivirus RNA replicon. In some embodiments, at least one of the one or more expression cassettes is operably positioned downstream to a transcriptional regulatory sequence (TRS) of the modified arterivirus RNA replicon, wherein the TRS is TRS1, TRS2, TRS3, TRS4, TRS5, TRS6, or TRS7. In some embodiments, at least one of the one or more expression cassettes further comprises a third nucleic acid sequence encoding one or more structural elements of a viral capsid enhancer, wherein the third nucleic acid sequence is operably linked upstream to the coding sequence for the GOI. In some embodiments, the nucleic acid sequence encoding the modified arterivirus RNA replicon further comprising a coding sequence for an autoprotease peptide operably linked downstream to the third nucleic acid sequence and upstream to the coding sequence for the GOI. In some embodiments, the coding sequence for the GOI encodes a therapeutic polypeptide, a prophylactic polypeptide, a diagnostic polypeptide, a nutraceutical polypeptide, an industrial enzyme, a reporter polypeptide, or any combination thereof. In some embodiments, the coding sequence for the GOI encodes an antibody, an antigen, an immune modulator, a cytokine, an enzyme, or any combination thereof.

In some embodiments, the modified viral RNA replicon comprises a modified RNA replicon derived from an alphavirus virus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), Salmonid alphavirus (SAV), and Buggy Creek virus. In some embodiments, the first nucleic acid sequence is operably positioned upstream to a nucleic acid sequence encoding one or more nonstructural proteins nsp1-4 or a portion thereof of the modified alphavirus RNA replicon.

In some embodiments, the nucleic acid sequence encoding the modified alphavirus RNA replicon further comprises one or more expression cassettes, wherein each of the expression cassettes comprises a promoter operably linked to a coding sequence for a gene of interest (GOI). In some embodiments, the modified alphavirus RNA replicon comprises at least two, three, four, five, or six expression cassettes. In some embodiments, at least one of the one or more expression cassettes is operably linked downstream of a nucleic acid sequence encoding one or more nonstructural proteins nsp1-4 or a portion thereof of the modified alphavirus RNA replicon. In some embodiments, at least one of the one or more expression cassettes further comprises a third nucleic acid sequence encoding one or more structural elements of a viral capsid enhancer, wherein the third nucleic acid sequence is operably linked upstream of the coding sequence for the GOI. In some embodiments, the modified alphavirus RNA replicon further comprising a coding sequence for an autoprotease peptide operably linked downstream to the third nucleic acid sequence and upstream to the coding sequence for the GOI. In some embodiments, the coding sequence for the GOI encodes a therapeutic polypeptide, a prophylactic polypeptide, a diagnostic polypeptide, a nutraceutical polypeptide, an industrial enzyme, a reporter polypeptide, or any combination thereof. In some embodiments, the coding sequence for the GOI encodes an antibody, an antigen, an immune modulator, a cytokine, an enzyme, or any combination thereof.

In another aspect, some embodiments disclosed herein relate to a method for conferring a resistance to the innate immune system in a subject, comprising administering to the subject a nucleic acid molecule comprising a nucleic acid sequence encoding a modified non-alphavirus RNA replicon, wherein the modified non-alphavirus RNA replicon comprises a first nucleic acid sequence encoding one or more structural elements of an alphavirus capsid enhancer and wherein expression of the modified non-alphavirus RNA replicon encoded by the nucleic acid molecule confers a resistance to innate immune response in the subject. In some embodiments, the subject is selected from the group consisting of human, horse, pig, primate, mouse, ferret, rat, cotton rat, cattle, swine, sheep, rabbit, cat, dog, bird, fish, goat, donkey, hamster, and buffalo.

Also disclosed herein include a method for producing a polypeptide of interest in a subject, where the method comprises administering to the subject a nucleic acid molecule comprising a nucleic acid sequence encoding a modified non-alphavirus RNA replicon, wherein the modified non-alphavirus RNA replicon comprises a first nucleic acid sequence encoding one or more structural elements of an alphavirus capsid enhancer. In some embodiments, the subject is human, horse, pig, primate, mouse, ferret, rat, cotton rat, cattle, swine, sheep, rabbit, cat, dog, bird, fish, goat, donkey, hamster, or buffalo.

Some embodiments disclosed herein relate to a method for producing a polypeptide of interest, where the method comprises culturing a host cell comprising a nucleic acid molecule which comprises a nucleic acid sequence encoding a modified non-alphavirus RNA replicon, wherein the modified non-alphavirus RNA replicon comprises a first nucleic acid sequence encoding one or more structural elements of an alphavirus capsid enhancer.

In some embodiments according to the above aspects of the disclosure, the modified non-alphavirus RNA replicon further comprising a second nucleic acid sequence encoding at least one nonstructural viral protein or a portion thereof, wherein the first nucleic acid sequence is operably linked upstream to the second nucleic acid sequence. In some embodiments, the modified non-alphavirus RNA replicon further comprises a coding sequence for an autoprotease peptide operably linked downstream to the first nucleic acid sequence and upstream to the second nucleic acid sequence. In some embodiments the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the modified non-alphavirus RNA replicon comprises a modified RNA replicon derived from a positive-strand RNA virus. In some embodiments, the modified non-alphavirus RNA replicon comprises a modified RNA replicon derived from a virus species belonging to Togaviridae family, Flaviviridae family, Orthomyxoviridae family, Rhabdoviridae family, or Paramyxoviridae family. In some embodiments, the modified non-alphavirus RNA replicon comprises a modified RNA replicon derived from a virus species belonging to the Arterivirus genus of the Arteriviridae family. In some embodiments, the sequence encoding the non-alphavirus modified RNA replicon further comprising one or more expression cassettes, wherein each of the expression cassettes comprises a promoter operably linked to a coding sequence for a gene of interest (GOI). In some embodiments, the modified non-alphavirus RNA replicon comprises at least two, three, four, five, or six expression cassettes. In some embodiments, at least one of the one or more expression cassettes is operably linked downstream of the second nucleic acid sequence encoding the at least one nonstructural viral protein or a portion thereof of the modified non-alphavirus RNA replicon. In some embodiments, at least one of the one or more expression cassettes further comprises a third nucleic acid sequence encoding one or more structural elements of an alphavirus capsid enhancer, wherein the third nucleic acid sequence is operably linked upstream to the coding sequence for the GOI. In some embodiments, the modified non-alphavirus RNA replicon further comprises a coding sequence for an autoprotease peptide operably linked downstream to the third nucleic acid sequence and upstream to the coding sequence for the GOI.

In some aspects, some embodiments disclosed herein relate to recombinant polypeptides produced by a method in accordance with one or more embodiments described herein.

Some embodiments disclosed herein relate to a composition including a recombinant polypeptide as described herein and a pharmaceutically acceptable carrier.

Some embodiments disclosed herein relate to a composition including a nucleic acid molecule as disclosed herein and a pharmaceutically acceptable carrier.

In some embodiments, one or more of the compositions and/or molecules of the present application, e.g. nucleic acid molecules, RNA replicons, and polypeptides, is further formulated into a pharmaceutical formulation. In some embodiments, one or more of the compositions and/or molecules of the present application is formulated into a pharmaceutical formulation with covalent compounds, non-covalent compounds, physical compositions, or pharmaceutically acceptable buffers.

In some embodiments disclosed herein, one or more of the compositions and/or molecules of the present application, e.g. nucleic acid molecules, RNA replicons, and polypeptides, is further formulated for use as a protective composition (e.g., vaccine) or therapeutic composition. In particular, protective compositions made in accordance with the present disclosure have a variety of uses including, but not limited to, use as vaccines and other therapeutic agents, use as diagnostic agents and use as antigens in the production of polyclonal or monoclonal antibodies.

The foregoing summary is illustrative only and is not intended to be in any way limiting. In addition to the illustrative embodiments and features described herein, further aspects, embodiments, objects and features of the application will become fully apparent from the drawings and the detailed description and the claims.

### BRIEF DESCRIPTION OF THE DRAWINGS

FIG. 1 is a graphical illustration of a non-limiting exemplary stem-loop RNA structure of an alphavirus capsid enhancer.

FIGS. 2A-2D are graphical representations of four non-limiting exemplary nucleic acid molecules of the present disclosure, where each of the nucleic acid molecules comprises a coding sequence for an alphavirus capsid enhancer (e.g., DLP motif) and a coding sequence for a gene of interest (GOI), e.g., a red Firefly (rFF) reporter gene. FIG. 2A: rEx-DLP-rFF; FIG. 2B: rEx-DLP-pp1ab-rFF; FIG. 2C: rEx-DLP-2A-pp1ab-rFF; and FIG. 2D: rEx-DLP-2A-pp1ab-DLP-rFF. DLP: Downstream Loop sequence; 2A: autoprotease peptide; pp1ab: nonstructural polypeptide sequence; and rFF: coding sequence for red Firefly reporter gene.

FIGS. 3A-3D are graphical illustrations of four non-limiting exemplary nucleic acid molecules of the present disclosure, where each of the nucleic acid molecules comprises a coding sequence for an alphavirus capsid enhancer (e.g., a DLP motif) and a coding sequence for a gene of interest (GOI), e.g., a red Firefly (rFF) reporter gene. FIG. 3A: Alpha-R-rFF; FIG. 3B: Alpha-R-DLP-rFF; FIG. 3C: Alpha-R-DLP-2A-nsp-rFF; and FIG. 3D: Alpha-R-DLP-2A-nsp-DLP-rFF. DLP: Downstream Loop sequence; 2A: autoprotease peptide; nsp1-4: nonstructural polypeptide sequence; and rFF: coding sequence for red Firefly reporter gene.

FIGS. 4A-4B are graphical illustrations of two other non-limiting exemplary nucleic acid molecules of the present disclosure, where each of the nucleic acid molecules comprises encoding coding sequence for an alphavirus capsid enhancer (e.g., a DLP motif) and a coding sequence for a gene of interest (GOI), e.g., a red Firefly (rFF) reporter gene. FIG. 4A: Alpha-R-DLP-2A-rFF; and FIG. 4B: Alpha-R-DLP-2A-nsp-DLP-2A-rFF. DLP: Downstream Loop sequence; 2A: autoprotease peptide; nsp1-4: nonstructural polypeptide sequence; and rFF: coding sequence for red Firefly reporter gene.

FIGS. 5A-5B graphically summarizes the results of flow cytometry analysis and bulk luciferase analyses performed to demonstrate that incorporating a DLP motif upstream of nucleic acid sequence encoding either EAV nonstructural protein genes or a gene of interest positioned in the subgenomic RNA, i.e. rFF reporter gene, did not negatively impact genomic RNA replication. In these experiments, FACS analysis (FIG. 5A) and bulk-cell luciferase assays (FIG. 5B) were carried out on electroporated cells.

FIGS. 6A-B graphically summarize the results of another exemplary flow cytometry analysis and bulk luciferase analysis performed to demonstrate that modified arterivirus replicon RNAs with a DLP motif incorporated upstream of the sequence encoding nonstructural protein genes can replicate and express efficiently in host cells that had been treated with IFN to induce the cellular innate immune system. In these experiments, FACS analysis (FIG. 6A) and bulk-cell luciferase assays (FIG. 6B) were carried out on electroporated cells. IFN was added to cell culture media five hours post electroporation. Samples were collected in triplicate eighteen hours post electroporation for analysis.

FIGS. 7A-C graphically summarizes the results of another exemplary bulk luciferase analysis performed to demonstrate that modified alphavirus replicon RNAs with a DLP motif incorporated upstream of the sequence encoding nonstructural protein genes can replicate and express efficiently in host cells that had been treated with IFN to induce the cellular innate immune system. In these experiments, bulk-cell luciferase assays were carried out on electroporated cells. IFN was added to cell culture media immediately after electroporation or three hours post electroporation. Samples were collected in triplicate eighteen hours post electroporation for analysis. FIG. 7A: α-rFF versus alpha-R-rFF construct; FIG. 7B: α-rFF versus α-DLP-2A-nsp-rFF; and FIG. 7C: α-rFF versus alpha-R-DLP-2A-nsp-rFF construct.

FIG. 8 graphically summarizes the results of exemplary in vivo experiments performed to demonstrate that modified alphavirus replicon RNAs with a DLP motif incorporated upstream of the sequence encoding nonstructural protein genes can replicate and express efficiently in Balb/c mice. In these experiments, whole body imaging of animals that had been injected with a modified alphavirus replicon RNA was conducted. Each animal received 7.5 μg of replicon RNA injected intramuscularly. Individual animals were imaged on day 1, day 3, and day 7. Original: mice injected with the alpha-R-rFF construct; DLP: mice injected with the alpha-R-DLP-2A-nsp-rFF construct.

FIG. 9 schematically depicts a non-limiting exemplary alphavirus genomic structure and genome expression (adapted from Strauss et al., Microbiological Reviews, pp. 491-562, September 1994). Genome organization of a Sindbis virus (SINV) is shown. The names of the nonstructural genes and structural protein genes are given. Referenced to the nomenclature of the genes and proteins can be found in Strauss et al., supra, 1994. The 49S genomic RNA is illustrated schematically in the center, with its translated ORF shown as an open box. Small black boxes are conserved sequence elements; the open diamond denotes the leaky opal termination codon. The nonstructural polyproteins and their processed products are shown above. Termination at the opal codon produces P123, whose major function in replication is believed to be as a proteinase that acts in trans to process the polyproteins in active RNA replicases; this proteinase domain is found in the nsP2 region. Read-through of the opal stop codon produces P1234, which can form an active replicase. The 26S subgenomic mRNA is expanded below to show the structural ORF and its translation products. Polypeptides present in the virion are shaded. vcRNA is the minus-strand complement of the genomic RNA.

FIG. 10 schematically depicts EAV genomic structure and genome expression strategy. The names of the replicase gene and structural protein genes are given (references to the nomenclature of genes and proteins can be found in Snijder et al., 2005). Below the genome organization, the structural relationships of the genome and sg mRNAs are depicted. The leader sequence and TRSs found at the 5′ end of the EAV mRNAs are indicated as blue and orange boxes, respectively. The ribosomal frameshifting element (RFS) found in the genome-length mRNA1 is indicated and the translated region of each mRNA is highlighted by a green line, whereas translationally silent regions are indicated by a red line. Only the translated open reading frames are indicated for each mRNA. The right-hand panels show a typical pattern of EAV mRNAs isolated from infected cells, visualized by hybridization to a probe complementary to the 3′ end of the genome and therefore recognizing all viral mRNA species.

FIGS. 11A-B schematically show the predicted stem-loop RNA structure of the 5′ CDS region of alphavirus mRNA 26S with a valley-peak topology. Two dimensional (2D) models of RNA structure based for the first 70-140 nucleotides of the CDS from seven representative Alphavirus mRNAs (SINV, SFV, RRV, SAGV, GETV, MIDV, UNAV, BEBV, MAYV and AURAV). The sequences were numbered from the initiation codon (AUGi), with A being the +1 position. The predicted structures are constructed based on SHAPE (selective 2′-hydroxyl acylation and primer extension) data (Toribio et al., 2016).

FIGS. 12A-C graphically summarize the results of exemplary in vivo experiments performed to demonstrate that modified alphavirus replicon RNAs with a DLP motif effect on immunogenicity in Balb/c mice. In this experiment, 6-8 week old BALB/c animals were primed at Days 0 and 42 using varying doses of the replicon RNA. Spleens and serum were collected on Day 56, and (a) flow cytometry for HA-specific T cell memory (CD8⁺CD44⁺CD62L^{Lo}KLRG-1^{Lo}IL-7Ra^{Hi}CXCR3^{Hi}) using Dextramers for detection (H-2 Kd [IYSTVASSL; SEQ ID NO: 44]) and (b,c) IFN-γ ELISpot to quantify CD8⁺ and CD4⁺ T cell effector responses. Statistics were one using multiple comparisons between matched doses using an ordinary one-way analysis of variance (ANOVA). FIG. 12A: A significant increase in memory precursor effector cells (MPECs) was observed in constructs containing the DLP motif compared with each comparable dose of unmodified replicon. FIG. 12B: Effector T cell responses were measured by the number of antigen-specific HA cells that were secreting IFN-γ following stimulation with a CD8+ T cell peptide. FIG. 12C: Effector T cell responses were measured by the number of antigen-specific HA cells that were secreting IFN-γ following stimulation with a CD4+ T cell peptide.

FIG. 13 graphically summarizes the results of exemplary in vivo experiments performed to demonstrate that modified alphavirus replicon RNAs with a DLP motif incorporated upstream of the sequence encoding nonstructural protein genes effectively prevent suppression of immune response upon pre-treatment with agents that simulate viral infection in Balb/c mic. 6-8 week old BALB/c animals were pre-treated with 20 μg of Poly(I:C) or saline administered via hydrodynamic tail vein injection 24 hours before vaccination to simulate an ongoing viral infection. Mice were then primed at Day 0 and boosted at Day 28 using a 1.5 μg dose of RNA replicon encoding HA. Serum was collected on Day 42, and HA-specific antibodies were measured in the serum. Serum antibody concentrations were calculated by interpolation of dilution versus optical density on a four-parametric logistic regression and using the 8D2 HA-specific monoclonal antibody as a standard. Statistics between individual groups were conducted using a Mann-Whitney (non-parametric) test.

FIGS. 14A-14C graphically summarize the results of in vivo experiments performed to demonstrate that the DLP-containing replicons according to the present disclosure are compatible with LNP (cationic lipid nanoparticle) formulations. In this experiment, 6-8 week old BALB/c animals were primed at Days 0 and boosted at Day 28 using varying doses of an RNA replicon encoding HA. Spleens and serum were collected on Day 42. FIG. 14A: HA-specific antibodies were measured in the serum. Serum antibody titer is the inverse of the EC20% and was calculated by interpolation of dilution versus optical density on a four-parametric logistic regression. FIG. 14B: IFN-γ ELISpot used to quantify CD8+ cell effector responses. For detection of antigen-specific CD8+ T cells, splenocytes were incubated with the H-2 Kd (IYSTVASSL; SEQ ID NO: 44) peptide. FIG. 14C: IFN-γ ELISpot used to quantify CD4+ T cell effector responses. For detection of antigen-specific CD4+ T cells, splenocytes were incubated with H2-D restricted CD4 T cell epitope KSSFFRNVVWLIKKN (SEQ ID NO: 45). Statistics between individual groups were conducted using a Mann-Whitney (non-parametric) test.

FIG. 15 graphically illustrates of a non-limiting exemplary configuration of DLP-containing mRNA, in which a Sindbis virus DLP element is placed upstream of a coding sequence for a gene of interest (GOI; dsGFP), and a 5′ UTR sequence is placed immediately downstream of a T7 promoter and upstream of the Sindbis virus DLP sequence. The coding sequence for dsGFP is linked to the DLP element via a P2A signal, which is an autocatalytic self-cleaving peptide (e.g., autoprotease peptide) derived from the porcine teschovirus-1. Also shown at the bottom portion of the figure is another non-limiting exemplary configuration of DLP-containing mRNA, in which a coding sequence for a destabilized form of EGFP reporter gene (dsGFP) used as a GOI is operably linked to the proteolytic PEST degradation signal derived from a mouse ornithine decarboxylase gene (MODC).

FIGS. 16A-D graphically summarize the results of experiments performed to demonstrate that DLP-containing modified mRNAs can confer interferon resistance. FIG. 16A: inclusion of DLP in mRNA results in a statistically significant increase in the frequency of GFP positive cells in the presence of IFN. Mean with 95% confidence intervals in Kruskai-Wallist test (non-parametric). FIG. 16B: unmodified mRNA is sensitive to IFN treatment (mean with 95% confidence intervals in 2-way ANOVA. Interaction: p=0.0083. Row: p=<0.0001. Column: p=0.0273. Sidak's multiple comparison test with * p=0.0217 and #p=<0.0241). FIG. 16C: DLP modified mRNA yields a statistically significant 30% increase in protein production per cell compared to unmodified mRNA in the presence of IFN (mean with 95% confidence intervals in 2-way ANOVA: p=<0.0001. Sidak's multiple comparison test with *** p=<0.0002 and **** p=<0.0001). FIG. 16D: DLP modified mRNA in the presence of IFN produces an equivalent amount of protein compared to unmodified mRNA in the absence of IFN treatment (mean with 95% confidence intervals in 2-way ANOVA. Interaction: p=<0.0001. Row: p=<0.0001. Column: p=0.0023. Sidak's multiple comparison test with **** p=<0.0001 and ** p=<0.0023).

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope; the disclosure will be described with additional specificity and detail through use of the accompanying drawings.

### DETAILED DESCRIPTION OF THE DISCLOSURE

The present disclosure generally relates to compositions and methods for use in regulating gene expression in cells. Some embodiments of the disclosure relate to expression systems, such as viral-based expression systems, with superior expression potential which are suitable for expressing heterologous molecules such as, for example, vaccines and therapeutic polypeptides, in recombinant cells. For example, some embodiments of the disclosure relate to nucleic acid molecules containing one or more structural elements of a viral capsid enhancer or a variant thereof. In some embodiments, at least one of the one or more structural elements comprises a RNA stem-loop. In some embodiments, at least one of the one or more structural elements is operably linked to a coding sequence of a gene of interest. Some embodiments of the disclosure relate to nucleic acid molecules such as transcription and/or expression constructs and vectors, containing a nucleic acid sequence encoding one or more structural elements of a viral capsid enhancer. Also disclosed herein in some embodiments are transcription vectors and expression vectors, such as viral-based vectors, comprising a coding sequence of a gene of interest. In some embodiments, the nucleic acid molecules of the present disclosure, e.g., messenger (mRNA) and RNA replicon, are generated via de novo synthesis and/or in vitro transcription. Recombinant cells that are genetically modified to include one or more of the nucleic acid molecules disclosed herein, as well as biomaterials and recombinant products derived from such cells are also within the scope of the application. Further provided herein are compositions and kits that include one or more of the nucleic acid molecules and/or recombinant cells disclosed herein, as well as methods for conferring a resistance to the innate immune system in a host cell.

In the following detailed description, reference is made to the accompanying drawings, which form a part hereof. In the drawings, similar symbols typically identify similar components, unless context dictates otherwise. The illustrative alternatives described in the detailed description, drawings, and claims are not meant to be limiting. Other alternatives may be used, and other changes may be made, without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of the present application.

Unless otherwise defined, all terms of art, notations and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Some Definitions

The singular form “a”, “an”, and “the” include plural references unless the context clearly dictates otherwise. For example, the term “a cell” includes one or more cells, comprising mixtures thereof.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms, “cells”, “cell cultures”, “cell line”, “recombinant host cells”, “recipient cells” and “host cells” as used herein, include the primary subject cells and any progeny thereof, without regard to the number of transfers. In some situations, a progeny is not exactly identical to the parental cell (due to deliberate or inadvertent mutations or differences in environment); however, such altered progeny is included in these terms, so long as the progeny retain the same or substantially similar functionality as that of the originally transformed cell.

As used herein, the term “construct” is intended to mean any recombinant nucleic acid molecule such as an expression cassette, plasmid, cosmid, fosmid, viral replicon, shuttle vector, autonomously replicating polynucleotide molecule, bacteriophage, or linear or circular, single-stranded or double-stranded, DNA or RNA polynucleotide molecule, derived from any source, capable of genomic integration or autonomous replication, comprising a nucleic acid molecule where nucleic acid sequences are linked in a functionally operative manner, e.g. operably linked.

The term “derived from” used herein refers to an origin or source, and may include naturally-occurring, recombinant, unpurified or purified molecules. The molecules of the present disclosure may be derived from viral or non-viral molecules. A protein or polypeptide derived from an original protein or polypeptide may include the original protein or polypeptide, in part or in whole, and may be a fragment or variant of the original protein or polypeptide.

The term “gene” is used broadly to refer to any segment of nucleic acid molecule that encodes a protein or that can be transcribed into a functional RNA. Genes may include sequences that are transcribed but are not part of a final, mature, and/or functional RNA transcript, and genes that encode proteins may further comprise sequences that are transcribed but not translated, for example, 5′ untranslated regions, 3′ untranslated regions, introns, etc. Further, genes may optionally further comprise regulatory sequences required for their expression, and such sequences may be, for example, sequences that are not transcribed or translated. Genes can be obtained from a variety of sources, including cloning from a source of interest or synthesizing from known or predicted sequence information, and may include sequences designed to have desired parameters.

The term “native” is used herein to refer to nucleic acid sequences or amino acid sequences as they naturally occur in the host. The term “non-native” is used herein to refer to nucleic acid sequences or amino acid sequences that do not occur naturally in the host, or are not configured as they are naturally configured in the host. A nucleic acid sequence or amino acid sequence that has been removed from a host cell, subjected to laboratory manipulation, and introduced or reintroduced into a host cell is considered “non-native.” Synthetic genes or partially synthetic genes introduced into a host cell or organism are “non-native.” Non-native genes further include genes endogenous to the host cell operably linked to one or more heterologous regulatory sequences that have been recombined into the host genome, or genes endogenous to the host cell or organism that are in a locus of the genome other than that where they naturally occur.

The terms “naturally-occurring” and “wild-type”, as used herein, refer to a form found in nature. For example, a naturally-occurring or wild-type nucleic acid molecule, nucleic acid sequence or protein may be present in and isolated from a natural source, and is not intentionally modified by human manipulation. As described in detail below, the nucleic acid molecules according to some embodiments of the present disclosure are non-naturally occurring nucleic acid molecules.

The term “heterologous” when used in reference to a polynucleotide, a gene, or a nucleic acid molecule refers to a polynucleotide, gene, or a nucleic acid molecule that is not derived from the host species. For example, “heterologous gene” or “heterologous nucleic acid sequence” as used herein, refers to a gene or nucleic acid sequence from a different species than the species of the host organism it is introduced into. When referring to a gene regulatory sequence such as, for example, an enhancer sequence, or to an auxiliary nucleic acid sequence used for manipulating expression of a gene sequence (e.g. a 5′ untranslated region, 3′ untranslated region, poly A addition sequence, etc.) or to a nucleic acid sequence encoding a protein domain or protein localization sequence, “heterologous” means that the regulatory or auxiliary sequence or sequence encoding a protein domain or localization sequence is from a different source than the gene with which the regulatory or auxiliary nucleic acid sequence or nucleic acid sequence encoding a protein domain or localization sequence is juxtaposed in a genome. Thus, a promoter operably linked to a gene to which it is not operably linked to in its natural state (for example, in the genome of a non-genetically engineered organism) is referred to herein as a “heterologous promoter,” even though the promoter may be derived from the same species (or, in some cases, the same organism) as the gene to which it is linked. For example, in some embodiments disclosed herein, a coding sequence of a heterologous gene of interest (GOI) is not linked to the recombinant RNA replicon sequence in its natural state. In some embodiments, the coding GOI sequence is derived from another organism, such as another virus, bacteria, fungi, human cell (tumor Ag), parasite (malaria), etc.)

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. Nucleic acid molecules can have any three-dimensional structure. A nucleic acid molecule can be double-stranded or single-stranded (e.g., a sense strand or an antisense strand). Non-limiting examples of nucleic acid molecules include genes, gene fragments, exons, introns, messenger RNA (mRNA), transfer RNA, ribosomal RNA, siRNA, micro-RNA, tracrRNAs, crRNAs, guide RNAs, ribozymes, cDNA, recombinant polynucleotides, branched polynucleotides, nucleic acid probes and nucleic acid primers. A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein. The nucleic acid molecules of the present disclosure can be synthesized ex vitro by any means known in the art, for example, using one or more chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules. In some embodiments, the nucleic acid molecules of the present disclosure are generated from de novo synthesis. In some embodiments, nucleic acid molecules can be synthesized de novo in whole or in part, using known chemical methods, known enzymatic techniques, or any combination thereof. For example, the component nucleic acid sequences can be synthesized by solid phase techniques, removed from the resin, and purified by preparative high performance liquid chromatography followed by chemical linkage and/or enzymatic ligation to form a chimeric nucleic acid molecule. The composition of the synthetic nucleic acid molecules may be confirmed by nucleic acid analysis or sequencing. In some embodiments, the nucleic acid molecules of the present disclosure can be enzymatically assembled from chemically synthesized oligonucleotides using techniques known in the art.

Nucleic acid molecules of the present disclosure can be nucleic acid molecules of any length, for example between about 0.5 Kb and about 1000 Kb, between about 0.5 Kb and about 500 Kb, between about 1 Kb and about 100 Kb, between about 2 Kb and about 50 Kb, or between about 5 Kb and about 20 Kb. In some embodiments, the nucleic acid molecule is, or is about, 0.5 Kb, 1 Kb, 2 Kb, 3 Kb, 4 Kb, 5 Kb, 6 Kb, 7 Kb, 8 Kb, 9 Kb, 10 Kb, 15 Kb, 20 Kb, 25 Kb, 30 Kb, 40 Kb, 50 Kb, 100 Kb, 200 Kb, 500 Kb, 1 Mb, or more, or a range between any two of these values.

The polynucleotides of the present disclosure can be “biologically active” with respect to either a structural attribute, such as the capacity of a nucleic acid to hybridize to another nucleic acid, or the ability of a polynucleotide sequence to be recognized and bound by one or more of a transcription factor, a ribosome, and a nucleic acid polymerase.

The term “recombinant” or “engineered” nucleic acid molecule as used herein, refers to a nucleic acid molecule that has been altered through human intervention. As non-limiting examples, a cDNA is a recombinant DNA molecule, as is any nucleic acid molecule that has been generated by ex vitro polymerase reaction(s), or to which linkers have been attached, or that has been integrated into a vector, such as a cloning vector or expression vector. As non-limiting examples, a recombinant nucleic acid molecule: 1) has been synthesized or modified ex vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature, 3) has been engineered using molecular cloning techniques such that it lacks one or more nucleotides with respect to the naturally-occurring nucleic acid molecule sequence, and/or 4) has been manipulated using molecular cloning techniques such that it has one or more sequence changes or rearrangements with respect to the naturally-occurring nucleic acid sequence. As non-limiting examples, a cDNA is a recombinant DNA molecule, as is any nucleic acid molecule that has been generated by ex vitro polymerase reaction(s), or to which linkers have been attached, or that has been integrated into a vector, such as a cloning vector or expression vector. In some embodiments disclosed herein, the recombinant nucleic acid molecules of the present application are generated from de novo synthesis.

The term “variant” of a protein used herein refers to a polypeptide having an amino acid sequence that is the same or essentially the same as that of the reference protein except having at least one amino acid modified, for example, deleted, inserted, or replaced, respectively. The amino acid replacement may be a conservative amino acid substitution, preferably at a non-essential amino acid residue in the protein. A “conservative amino acid substitution” is one in which the amino acid residue is replaced with an amino acid residue having a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A variant of a protein may have an amino acid sequence at least about 80%, 90%, 95%, or 99%, preferably at least about 90%, more preferably at least about 95%, identical to the amino acid sequence of the protein. Preferably, a variant is a functional variant of a protein that retains the same function as the protein. The terms “variant”, when used in reference to a nucleic acid sequence, refer to a nucleic acid sequence that differs by one or more nucleotides from another, usually related nucleotide acid sequence. As such, the term “variant” can refer to a change of one or more nucleotides of a reference nucleic acid which includes the insertion of one or more new nucleotides, deletion of one or more nucleotides, and substitution of one or more existing nucleotides. A “variation” is a difference between two different nucleotide sequences; typically, one sequence is a reference sequence. Broadly, the term “nucleotide variation” as used herein includes point mutation, multiple mutation, single nucleotide polymorphism (SNP), deletion, insertion, and translocation. The term “reference nucleic acid” is used herein to describe a nucleotide sequence having a known reference sequence of interest.

As used herein, the terms, “identical” or percent “identity”, in the context of two or more nucleic acids or polypeptide sequences, refer to two or more sequences or subsequences that are the same or have a specified percentage of amino acid residues or nucleotides that are the same, when compared and aligned for maximum correspondence over a comparison window. Unless otherwise specified, the comparison window for a selected sequence, e.g., “SEQ ID NO: X” is the entire length of SEQ ID NO: X, and, e.g., the comparison window for “100 bp of SEQ ID NO: X” is the stated 100 bp. The degree of amino acid or nucleic acid sequence identity can be determined by various computer programs for aligning the sequences to be compared based on designated program parameters. For example, sequences can be aligned and compared using the local homology algorithm of Smith & Waterman Adv. Appl. Math. 2:482-89, 1981, the homology alignment algorithm of Needleman & Wunsch J. Mol. Biol. 48:443-53, 1970, or the search for similarity method of Pearson & Lipman Proc. Nat'l. Acad. Sci. USA 85:2444-48, 1988, and can be aligned and compared based on visual inspection or can use computer programs for the analysis (for example, GAP, BESTFIT, FASTA, and TFASTA in the Wisconsin Genetics Software Package, Genetics Computer Group, 575 Science Dr., Madison, WI).

In addition to calculating percent sequence identity, the BLAST algorithm also performs a statistical analysis of the similarity between two sequences (see, e.g., Karlin & Altschul, Proc. Nat'l. Acad. Sci. USA 90:5873-87, 1993). The smallest sum probability (P(N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, preferably less than about 0.01, and more preferably less than about 0.001.

As used herein, the term “vector” refers to a recombinant polynucleotide construct designed for transfer to a host cell, or between host cells, and that may be used for the purpose of transformation, e.g. the introduction of heterologous DNA into a host cell. A vector can be, for example a replicon, such as a plasmid, bacteriophage, or cosmid, into which another DNA segment may be inserted so as to bring about the replication of the inserted segment. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments, for example ex vitro, ex vivo, and in vivo. In some embodiments, the vector is a plasmid, a bacteriophage vector, a cosmid, a fosmid, a viral replicon, or a combination thereof. In some embodiments, the vector is a eukaryotic vector, a prokaryotic vector (e.g., a bacterial plasmid), or a shuttle vector. An expression system can be, for example, an expression vector or an expression cassette. In some embodiments, the vector is a transcription vector. The term “transcription vector” refers to a vector capable of being transcribed but not translated. For example, transcription vectors can be used to amplify their insert.

Virus-based “replicon” expression vectors can be used as, for example, vaccines and therapeutic compositions. Replicon vectors may be utilized in several formats, including DNA, RNA, and recombinant viral particles. A wide body of literature has now demonstrated efficacy of viral replicon vectors for applications such as vaccines. Moreover, these terms may be referred to collectively as vectors, vector constructs or gene delivery vectors.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

### Viral Capsid Enhancers

Some viruses have sequences capable of forming one or more stem-loop structures which regulate, for example increase, capsid gene expression. The term “viral capsid enhancer” is used herein to refer to a regulatory element comprising sequences capable of forming such stem-loop structures. In some examples, the stem-loop structures are formed by sequences within the coding sequence of a capsid protein and named Downstream Loop (DLP) sequence. As disclosed herein, these stem-loop structures or variants thereof can be used to regulate, for example increase, expression level of genes of interest. For example, these stem-loop structures or variants thereof can be used in a recombinant vector (e.g., in a heterologous viral genome) for enhancing transcription and/or translation of coding sequence operably linked downstream thereto. As an example, members of the Alphavirus genus can resist the activation of antiviral RNA-activated protein kinase (PKR) by means of a prominent RNA structure present within in viral 26S transcripts, which allows an eIF2-independent translation initiation of these mRNAs. This structure, called the downstream loop (DLP), is located downstream from the AUG in SINV 26S mRNA and in other members of the Alphavirus genus. In the case of Sindbis virus, the DLP motif is found in the first ˜150 nt of the Sindbis subgenomic RNA. The hairpin is located downstream of the Sindbis capsid AUG initiation codon (AUG is collated at nt 50 of the Sindbis subgenomic RNA). Previous studies of sequence comparisons and structural RNA analysis revealed the evolutionary conservation of DLP in SINV and predicted the existence of equivalent DLP structures in many members of the Alphavirus genus (see e.g., Ventoso, J. Virol. 9484-9494, Vol. 86, September 2012).

PKR phosphorylates the eukaryotic translation initiation factor 2α (eIF2α). Phosphorylation of eIF2α blocks translation initiation of mRNA and in doing so keeps viruses from a completing a productive replication cycle. PKR is activated by interferon and double stranded RNA. Alphavirus replication in host cells is known to induce the double-stranded RNA-dependent protein kinase (PKR). For example, Sindbis virus infection of cells induces PKR that results in phosphorylation of eIF2α yet the viral subgenomic mRNA is efficiently translated while translation of all other cellular mRNAs is restricted. The subgenomic mRNA of Sindbis virus has a stable RNA hairpin loop located downstream of the wild type AUG initiator codon for the virus capsid protein (e.g., capsid enhancer). This hairpin loop, also called stem-loop, RNA structure is often referred to as the Downstream LooP structure (or DLP motif). It has been reported that the DLP structure can stall a ribosome on the wild type AUG and this supports translation of the subgenomic mRNA without the requirement for functional eIF2α. Thus, subgenomic mRNAs of Sindbis virus (SINV) as well as of other alphaviruses are efficiently translated even in cells that have highly active PKR resulting in complete phosphorylation of eIF2a.

### Structure of Alphavirus DLPs

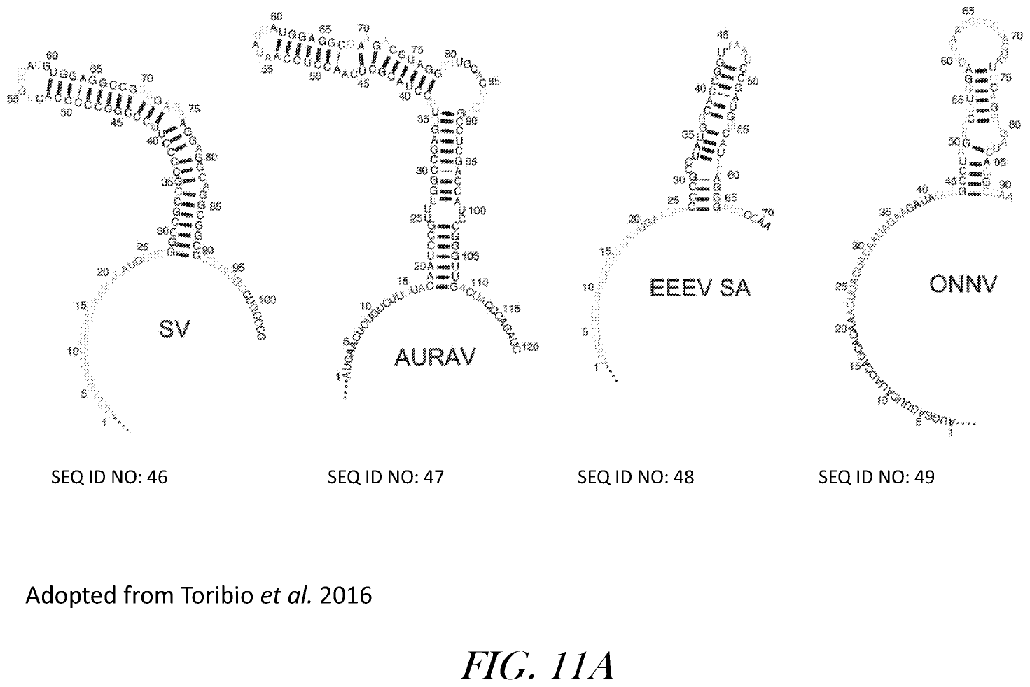

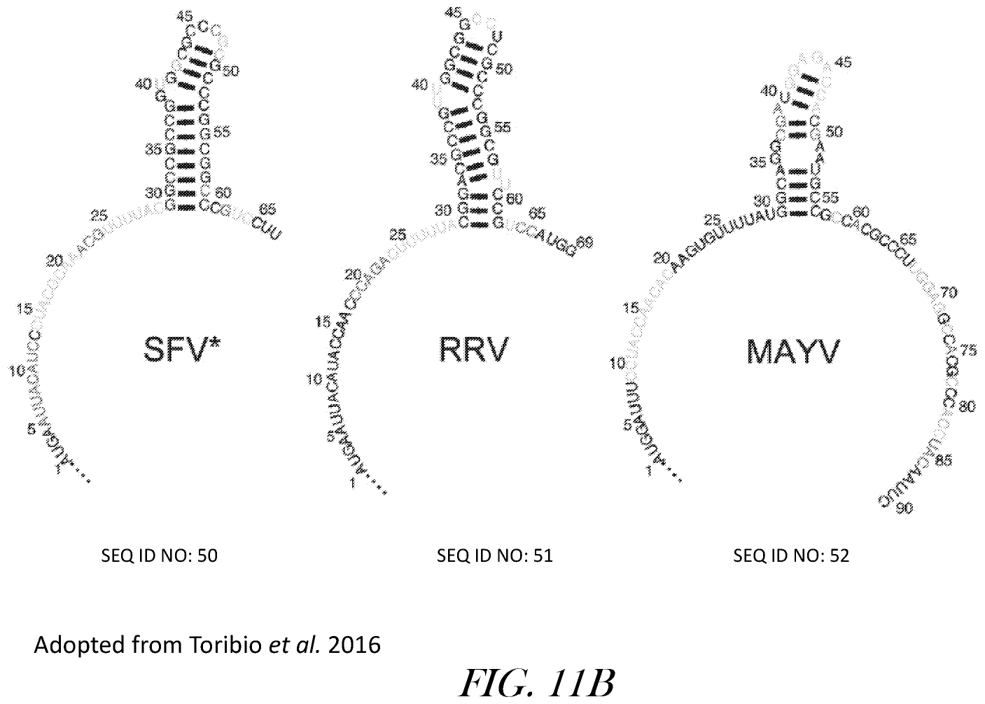

The DLP structure was first characterized in Sindbis virus (SINV) 26S mRNA and also detected in Semliki Forest virus (SFV). Similar DLP structures have been reported to be present in at least 14 other members of the Alphavirus genus including New World (for example, MAYV, UNAV, EEEV (NA), EEEV (SA), AURAV) and Old World (SV, SFV, BEBV, RRV, SAG, GETV, MIDV, CHIKV, and ONNV) members. The predicted structures of these Alphavirus 26S mRNAs were constructed based on SHAPE (selective 2′-hydroxyl acylation and primer extension) data (Toribio et al., Nucleic Acids Res. May 19; 44(9):4368-80, 2016), the content of which is hereby incorporated by reference). Stable stem-loop structures were detected in all cases except for CHIKV and ONNV, whereas MAYV and EEEV showed DLPs of lower stability (see FIGS. 11A-B and Toribio et al., 2016 supra). The highest DLP activities were reported for those Alphaviruses that contained the most stable DLP structures. In some instances, DLP activity depends on the distance between the DLP motif and the initiation codon AUG (AUGi). The AUG-DLP spacing in Alphavirus 26S mRNAs is tuned to the topology of the ES6S region of the ribosomal 18S rRNA in a way that allows the placement of the AUGi in the P site of the 40S subunit stalled by the DLP, allowing the incorporation of Met-tRNA without the participation of eIF2. Two main topologies were detected: a compact and stable structure in the SFV clade, and a more extended structure in the SINV group. In both cases, it was observed that DLP structures were preceded by a region of intense SHAPE reactivity, suggesting a single stranded conformation for the AUG-DLP stretch. Accordingly, this region showed a high content of A and a low content of G that resulted in a low propensity to form secondary structures when compared with equivalent positions in whole mouse mRNA transcriptome or in those Alphavirus mRNAs lacking DLPs. These results reported by Toribio et al. (2016, supra) suggest that the occurrence of DLPs in Alphavirus is probably linked to a flattening of the preceding region, resulting in a valley-peak topology for this region of mRNA.

In the case of Sindbis virus, the DLP motif is found in the first ˜150 nt of the Sindbis subgenomic RNA. The hairpin is located downstream of the Sindbis capsid AUG initiation codon (AUG at nt 50 of the Sindbis subgenomic RNA) and results in stalling a ribosome such that the correct capsid gene AUG is used to initiate translation. This is because the hairpin causes ribosomes to pause eIF2a is not required to support translation initiation. Without being bound by any particular theory, it is believed that placing the DLP motif upstream of a coding sequence for any GOI typically results in a fusion-protein of N-terminal capsid amino acids that are encoded in the hairpin region to the GOI encoded protein because initiation occurs on the capsid AUG not the GOI AUG. In some embodiments disclosed herein, a porcine teschovirus-1 2A (P2A) peptide sequence was engineered in-frame immediately after the DLP sequence and in-frame immediately upstream of all GOI. The incorporation of the P2A peptide in the modified viral RNA replicons of the present disclosure allows release of a nearly pristine GOI protein from the capsid-GOI fusion; a single proline residue is added to all GOI proteins.

Without being bound by any particular theory, it is believed that the DLP allows translation to occur in an eIF2a independent manner, nucleic acid molecules and expression vectors (e.g., RNA replicon vectors) engineered to use it to initiate translation of non-structural proteins have increased functionality in cells that are innate immune system activated. Therefore, it is contemplated that DLP-engineered nucleic acid molecules and expression vectors (e.g., RNA replicon vectors) also function with more uniformity in different cells, individuals or populations of individuals because differences in the level of innate immune activation in each will naturally cause variability. In some embodiments, the DLP can assist in removing that variability because translation and replication of RNA replicon vectors (as well as GOI expression) can be less impacted by pre-existing innate immune responses. One of the significant values of the compositions and methods disclosed herein is that vaccine efficacy can be increased in individuals that are in a chronic or acute state of immune activation. Causes of chronic or acute immune activation could be found in individuals suffering from a subclinical or clinical infection or individuals undergoing medical treatments for cancer or other maladies (e.g., diabetes, malnutrition, high blood pressure, heart disease, Crohn's disease, muscular scleroses, etc.).

As described herein, DLP-containing nucleic acid molecules (for example, transcription and expression vectors (e.g., RNA viral replicons)) disclosed herein can be useful in conferring a resistance to the innate immune system in a subject. Unmodified RNA replicons are sensitive to the initial innate immune system state of cells they are introduced into. If the cells/individuals are in a highly active innate immune system state, the RNA replicon performance (e.g., replication and expression of a GOI) can be negatively impacted. By engineering a DLP to control initiation of protein translation, particularly of non-structural proteins, the impact of the pre-existing activation state of the innate immune system to influence efficient RNA replicon replication is removed or lessened. The result is more uniform and/or enhanced expression of a GOI that can impact vaccine efficacy or therapeutic impact of a treatment.

### Arteriviruses

The arteriviruses (Family Arteriviridae, Genus Arterivirus) encompass an important group of enveloped, single-stranded, positive-sense RNA viruses which infect domestic and wild animals. Arteriviruses share a similar genome organization and replication strategy to that of members of the family Coronaviridae (genera Coronavirus and Torovirus), but differ considerably in their genetic complexity, genome length, biophysical properties, size, architecture, and structural protein composition of the viral particles (e.g., virion). Currently, the Arterivirus genus is considered to include equine arteritis virus (EAV), porcine reproductive and respiratory syndrome virus (PRRSV), lactate dehydrogenase-elevating virus (LDV) of mice, simian hemorrhagic fever virus (SHFV), and wobbly possum disease virus (WPDV).

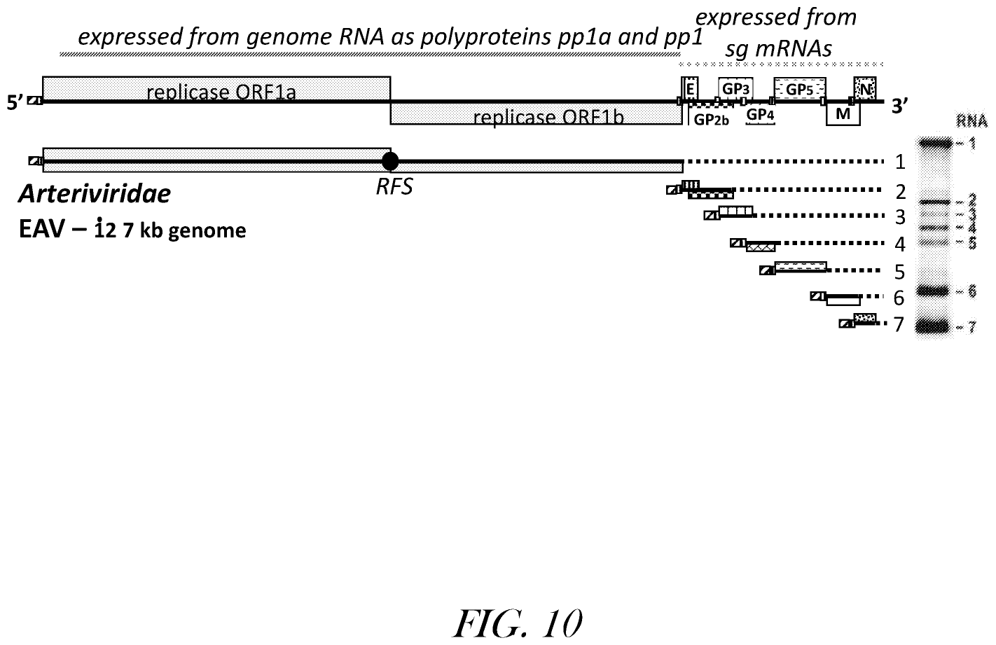

A typical arterivirus genome varies between 12.7 and 15.7 kb in length but their genome organization is relatively consistent with some minor variations. Exemplary genome organization and virion architecture of an arterivirus is shown in FIG. 10. The arterivirus genome is a polycistronic +RNA, with 5′ and 3′ non-translated regions (NTRs) that flank an array of 10-15 known ORFs. The large replicase ORFs 1a and 1b occupy the 5′-proximal three-quarters of the genome, with the size of ORF1a being much more variable than that of ORF1b. Translation of ORF1a produces replicase polyprotein (pp) 1a, whereas ORF1b is expressed by −1 programmed ribosomal frameshifting (PRF), which C-terminally extends ppla into pp1ab. In addition, a short transframe ORF has been reported to overlap the nsp2-coding region of ORF1a in the +1 frame and to be expressed by −2 PRF. The 3′-proximal genome part has a compact organization and contains 8 to 12 relatively small genes, most of which overlap with neighboring genes. These ORFs encode structural proteins and are expressed from a 3′-co-terminal nested set of sg mRNAs. The organization of these ORFs is conserved, but downstream of ORF1b, SHFV and all recently identified SHFV-like viruses contain three or four additional ORFs (˜1.6 kb) that may be derived from an ancient duplication of ORFs 2-4. Together with the size variation in ORF1a, this presumed duplication explains the genome size differences among arteriviruses.

With regard to equine arteritis virus (EAV), the wild-type EAV genome is approximately 12.7 Kb in size. The 5′ three fourths of the genome codes for two large replicase proteins 1a and 1ab; the amino acid sequences of the two proteins are N-terminally identical but due to a ribosomal frameshift the amino acid sequence of the C-terminal region of 1ab is unique. The 3′ one quarter of the EAV genome codes for the virus's structural protein genes, all of which are expressed from subgenomic RNAs. The subgenomic RNAs form a nested set of 3′ co-terminal RNAs that are generated via a discontinuous transcriptional mechanism. The subgenomic RNAs are made up of sequences that are not contiguous with the genomic RNA. All of the EAV subgenomic RNAs share a common 5′ leader sequence (156 to 221 nt in length) that is identical to the genomic 5′ sequence. The leader and body parts of the subgenomic RNAs are connected by a conserved sequence termed a transcriptional-regulatory sequence (TRS). The TRS is found on the 3′ end of the leader (leader TRS) as well as in the subgenomic promoter regions located upstream of each structural protein gene (body TRS). Subgenomic RNAs are generated as the negative strand replication intermediate RNA is transcribed. As transcription occurs the replication complex pauses as it comes to each body TRS and then the nascent negative strand RNA become associated with the complementary positive strand leader TRS where negative strand RNA transcription continues. This discontinuous transcription mechanism results in subgenomic RNA with both 5′ and 3′ EAV conserved sequences. The negative strand subgenomic RNAs then become the template for production of the subgenomic positive sense mRNA.

Infectious cDNA clones, representing the entire genome of EAV, have been reported and they have been used to study EAV RNA replication and transcription for nearly two decades. In addition, infectious clones have been generated that contain the chloramphenicol acetyltransferase (CAT) gene inserted in place of ORF2 and ORF7, and CAT protein was shown to be expressed in cells electroporated with those RNAs. Modifications of the infectious clone via site directed mutagenesis and deletion of the structural protein gene regions has been used to determine the requirement for each structural gene in support of RNA replication (Molenkamp 2000). The study reported by Molenkamp 2000 concluded that the structural genes are not required to support RNA replication. Analysis of sequence homology requirements for TRS activity in subgenomic RNA production was conducted and used to better define how discontinuous transcription mechanistically occurs (van Marle 1999, Pasternak 2000, Pasternak 2001, Pasternak 2003, van den Born 2005) and defective interfering RNAs have been used to understand the minimal genomic sequences required for replication and packaging of RNA into virus particles (Molenkamp 2000a).

### Alphaviruses

Alphavirus is a genus of genetically, structurally, and serologically related viruses of the group IV Togaviridae family which includes at least 30 members, each having single stranded RNA genomes of positive polarity enclosed in a nucleocapsid surrounded by an envelope containing viral spike proteins. Currently, the alphavirus genus comprises among others the Sindbis virus (SIN), the Semliki Forest virus (SFV), the Ross River virus (RRV), Venezuelan equine encephalitis virus (VEEV), and Eastern equine encephalitis virus (EEEV), which are all closely related and are able to infect various vertebrates such as mammal, rodents, fish, avian species, and larger mammals such as humans and horses as well as invertebrates such as insects. Transmission between species and individuals occurs mainly via mosquitoes making the alphaviruses a contributor to the collection of Arboviruses—or Arthropod-Borne Viruses. In particular, the Sindbis and the Semliki Forest viruses have been widely studied and, therefore, the life cycle, mode of replication, etc., of these viruses are well characterized. In particular, alphaviruses have been shown to replicate very efficiently in animal cells which makes them valuable as vectors for production of protein and nucleic acids in such cells.

Alphavirus particles are enveloped, have a 70 nm diameter, tend to be spherical (although slightly pleomorphic), and have an approximately 40 nm isometric nucleocapsid. FIG. 9 depicts a typical alphavirus genomic structure and genome expression. Alphavirus genome is single-stranded RNA of positive polarity of approximately 11-12 kb in length, comprising a 5′ cap, a 3′ poly-A tail, and two open reading frames with a first frame encoding the nonstructural proteins with enzymatic function and a second frame encoding the viral structural proteins (e.g., the capsid protein C, E1 glycoprotein, E2 glycoprotein, E3 protein and 6K protein).

The 5′ two-thirds of the alphavirus genome encodes a number of nonstructural proteins necessary for transcription and replication of viral RNA. These proteins are translated directly from the RNA and together with cellular proteins form the RNA-dependent RNA polymerase essential for viral genome replication and transcription of subgenomic RNA. Four nonstructural proteins (nsP1-4) are produced as a single polyprotein which constitutes the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises subgenomic RNA which serves as a template for translation of all the structural proteins required for forming viral particles: the core nucleocapsid protein C, and the envelope proteins P62 and E1 that associate as a heterodimer. The viral membrane-anchored surface glycoproteins are responsible for receptor recognition and entry into target cells through membrane fusion. The subgenomic RNA is transcribed from the p26S subgenomic promoter present at the 3′ end of the RNA sequence encoding the nsp4 protein. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphaviral glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses lose the peripheral protein E3, while in Semliki viruses it remains associated with the viral surface.

Alphavirus replication has been reported to take place on membranous surface within the host cell. In the first step of the infectious cycle, the 5′ end of the genomic RNA is translated into a polyprotein (nsP1-4) with RNA polymerase activity that produces a negative strand complementary to the genomic RNA. In a second step, the negative strand is used as a template for the production of two RNAs, respectively: (1) a positive genomic RNA corresponding to the genome of the secondary viruses producing, by translation, other nsp proteins and acting as a genome for the virus; and (2) subgenomic RNA encoding the structural proteins of the virus forming the infectious particles. The positive genomic RNA/subgenomic RNA ratio is regulated by proteolytic autocleavage of the polyprotein to nsp 1, nsp 2, nsp 3 and nsp 4. In practice, the viral gene expression takes place in two phases. In a first phase, there is main synthesis of positive genomic strands and of negative strands. During the second phase, the synthesis of subgenomic RNA is virtually exclusive, thus resulting in the production of large amount of structural protein.

### Innate Immunity

Since innate immune activation can occur due to many different stimuli, vaccine approaches that rely on self-amplifying RNA replicons to express antigen or therapeutic GOI can be negatively impacted by the global host protein shutdown associated with PKR phosphorylation of eIF2a. Engineering RNA replicons to function in a cellular environment where host protein translation is repressed would provide those systems with a significant advantage over standard RNA replicon systems.

Accordingly, RNA replicon systems that are negatively impacted by innate immune responses, such as systems derived from alphaviruses and arteriviruses, can be more effective at expressing their encoded GOI when engineered to contain a DLP motif. The DLP motif confers efficient mRNA translation in cellular environments where cellular mRNA translation is inhibited. When a DLP is linked with translation of a replicon vectors non-structural protein genes the replicase and transcriptase proteins are capable of initiating functional replication in PKR activated cellular environments. When a DLP is linked with translation of subgenomic mRNAs robust GOI expression is possible even when cellular mRNA is restricted due to innate immune activation. Accordingly, engineering replicons that contain DLP structures to help drive translation of both non-structural protein genes and subgenomic mRNAs provides yet another powerful way to overcome innate immune activation.

Some embodiments of the disclosure relate to DLP structures that have been engineered to support translation of viral non-structural genes of replicon vectors derived from two different viruses, Venezuelan equine encephalitis virus (VEEV) and equine arteritis virus (EAV), thus conveying innate immune response evasion to the systems. As described in greater detail below, incorporation of the DLP structures into the replicon vectors made them both resistant to interferon (IFN) treatment and unexpectedly also resulted in an overall increase in GOI expression potential. The combination of IFN resistance and superior protein expression potential imparted by engineering a DLP into the RNA replicon systems make them suitable for use in individuals or populations where innate immune activation is acutely or chronically present.

### Nucleic Acid Molecules of the Disclosure

Some aspects of the present disclosure relate to nucleic acid molecules, such as synthetic or recombinant nucleic acid molecules, that include one or more DLP motifs, a coding sequence for one or more DLP motifs, or a combination thereof. In some embodiments, the nucleic acid molecules of the disclosure can include a coding sequence for a gene of interest (GOI) operably linked to DLP motif(s) and/or the coding sequence for the DLP motifs.

In one aspect, disclosed herein is a nucleic acid molecule, comprising (i) a first nucleic acid sequence encoding one or more structural elements of a viral capsid enhancer or a variant thereof; and (ii) a second nucleic acid sequence operably linked to the first nucleic acid sequence, wherein the second nucleic acid sequence comprises a coding sequence for a gene of interest (GOI). In some embodiments, at least one of the one or more structural elements of the viral capsid enhancer comprises one or more RNA stem-loops. In some embodiments, at least one of the one or more RNA stem-loops is comprised by a DLP motif present in the first nucleic acid sequence. In some embodiments, at least one of the one or more structural elements of the viral capsid enhancer does not comprise any RNA stem-loop.

As described above, a viral capsid enhancer comprises sequences within the 5′ non-coding and/or 5′ coding sequences (preferably, the 5′ coding sequences) of that enhance expression (e.g., transcription and/or translation) of sequences operably linked therewith. In some embodiments of the present disclosure, the one or more structural elements of the viral capsid enhancer include one or two RNA stem-loops of the viral capsid enhancer. In some embodiments, the viral capsid enhancer of the present disclosure includes the sequences containing the 26S subgenomic promoter. In some embodiments, the viral capsid enhancer of the disclosure contains the 5′ coding sequences at about nucleotides 20 to 250, about nucleotides 20 to 200, about nucleotides 20 to 150, about nucleotides 20 to 100, or about nucleotides 50 to 250, about nucleotides 100 to 250, about nucleotides 50 to 200, about nucleotides 75 to 250, about nucleotides 75 to 200, about nucleotides 75 to 150, about nucleotides 77 to 139, or about nucleotides 100 to 250, about nucleotides 150 to 250, about nucleotides 100 to 150, about nucleotides 100 to 200 of the viral 26S RNA, which is capable of forming a hairpin structure. In some embodiments, the first nucleic acid sequence encoding one or more structural elements of a viral capsid enhancer that are important for enhancing expression of a heterologous sequence operably linked thereto. In some embodiments, the first nucleic acid sequence includes encoding sequence for one or more RNA stem-loops of a viral capsid enhancer. In some embodiments, the first nucleic acid sequence encoding one or more structural elements of a viral capsid enhancer that are important for enhancing translation of a heterologous sequence operably linked thereto. In some embodiments, the first nucleic acid sequence encoding one or more structural elements of a viral capsid enhancer that are important for enhancing transcription of a heterologous sequence operably linked thereto.

In some embodiments, the first nucleic acid sequence of the nucleic acid molecule includes at least about 50, about 75, about 100, about 150, about 200, about 300, or more nucleotides from the 5′ coding sequence for a viral capsid protein. In some embodiments, the first nucleic acid sequence of the nucleic acid molecule includes about 50, about 75, about 100, about 150, about 200, about 300, or more, or a range between any two of these values, nucleotides from the 5′ coding sequence for a viral capsid protein. In some embodiments, the viral capsid enhancer is derived from a capsid gene of an alphavirus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus. In some embodiments, the viral capsid enhancer is derived from a capsid gene of a Sindbis virus species or a Semliki Forest virus species. In some particular embodiments, the viral capsid enhancer is derived from a capsid gene of a Sindbis virus species. Additionally, one of ordinary skill in the art will appreciate that modifications may be made in the 5′ coding sequences from the viral capsid protein without substantially reducing its enhancing activities. More information in this regard can be found in, e.g., Frolov et al., J. Virology 70:1182, 1994; Frolov et al., J. Virology 68:8111, 1994. In some embodiments, it can be advantage for such mutations to substantially preserve the RNA hairpin structure formed by the 5′ capsid coding sequences.

In some embodiments, the viral capsid enhancer disclosed herein does not contain one or more, or all, of the 5′ coding sequences of the capsid protein that are upstream of the hairpin structure. In some embodiments, the viral capsid enhancer disclosed herein does not contain all of the 5′ coding sequences of the viral capsid protein that are upstream of the hairpin structure. In some embodiments, the viral capsid enhancer sequence may encode all or part of the capsid protein. Accordingly, in some embodiments disclosed herein, the capsid enhancer region will not encode the entire viral capsid protein. In some embodiments, the viral capsid enhancer sequence encodes an amino terminal fragment from the viral capsid protein. In those embodiments in which an otherwise functional capsid protein is encoded by the capsid enhancer sequence, it may be desirable to ablate the capsid autoprotease activity. Capsid mutations that reduce or ablate the autoprotease activity of the capsid protein are known in the art (see e.g., WO1996/37616). In addition or alternatively, one or more of amino acid residues in the capsid protein may be altered to reduce capsid protease activity.

As indicated above, previous studies of sequence comparisons and structural RNA analysis revealed the evolutionary conservation of DLP motifs in many members of the Alphavirus genus (see e.g., Ventoso, 2012 supra). Accordingly, in some further embodiments, the viral capsid enhancer sequence of the present disclosure can be of any other variant sequence such as, for example, a synthetic sequence or a heterologous sequence, that can form an RNA hairpin functionally or structurally equivalent to one or more of the RNA stem-loops predicted for a viral capsid enhancer and which can act to enhance translation of RNA sequences operably linked downstream thereto (e.g., coding sequence for a gene of interest). Non-limiting examples of RNA stem-loops which can act as a transcriptional and/or translational enhancer include those shown in FIGS. 11A-B. In some embodiments, the nucleic acid molecule of the disclosure includes an alphavirus capsid enhancer as derived from Sindbis virus (SINV; NC 001547.1), Aura virus (AURAV; AF126284), Chikungunya virus (CHIKV; NC 004162), O'Nyong-Nyong virus (ONNV; NC 001512), Eastern Equine Encephalitis virus (EEEV(SA); AF159559 and EEEV (NA); U01558), Mayaro virus (MAYV; DQ001069), Semliki Forest virus (SFV; NC 003215), Ross River virus (RRV; DQ226993 and Sagiyama virus (SAGV; AB032553), Getah virus (GETV; NC 006558), Middelburg virus (MIDV; EF536323), Una virus (UNAV; AF33948), or Bebaru virus (BEBV; AF339480) as described in Toribio et al., 2016 supra, the content of which is hereby incorporated by reference in its entirety, or a variant thereof.

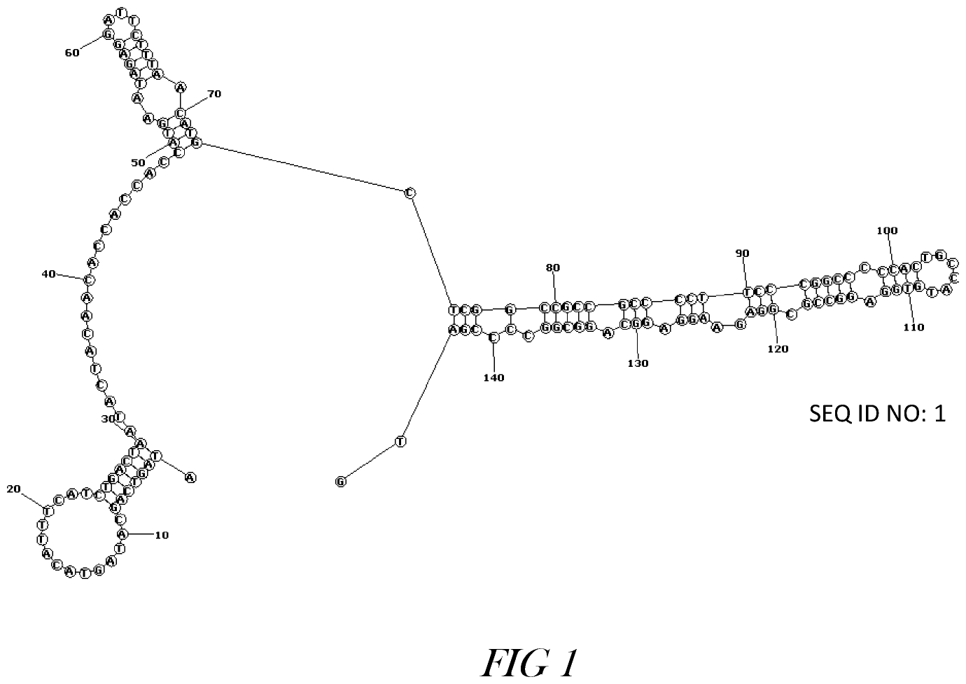

Nucleic acid molecules having a high degree of sequence identity (e.g., at least 85%, at least 90%, at least 95%, at least 96%, at least 97%, at least 98%, at least 99%, or 100% sequence identity) to the coding sequence for a viral capsid enhancer disclosed herein can be identified and/or isolated by using the sequence described herein (e.g., SEQ ID NO: 1) or any others alphavirus capsid protein as they are known in the art, for example, by using the sequences of Sindbis virus (SINV; NC 001547.1), Aura virus (AURAV; AF126284), Chikungunya virus (CHIKV; NC 004162), O'Nyong-Nyong virus (ONNV; NC 001512), Eastern Equine Encephalitis virus (EEEV(SA); AF159559 and EEEV (NA); U01558), Mayaro virus (MAYV; DQ001069), Semliki Forest virus (SFV; NC 003215), Ross River virus (RRV; DQ226993 and Sagiyama virus (SAGV; AB032553), Getah virus (GETV; NC 006558), Middelburg virus (MIDV; EF536323), Una virus (UNAV; AF33948), and Bebaru virus (BEBV; AF339480), by genome sequence analysis, hybridization, and/or PCR with degenerate primers or gene-specific primers from sequences identified in the respective alphavirus genome. For example, the viral capsid enhancer can comprise, or consist of, a DLP motif from a virus species belonging to the Togaviridae family, for example an alphavirus species or a rubivirus species. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the 5′ CDS portion of an alphavirus capsid protein. In some embodiments, the 5′ CDS portion of an alphavirus capsid protein comprises at least the first 25, 50, 75, 80, 100, 150, or 200 nucleotides of the coding sequence for the alphavirus capsid protein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the nucleic acid sequence of any one of SEQ ID NOs: 1 and 46-52. In some embodiments, the nucleic acid molecule comprises a viral capsid enhancer having a nucleic acid sequence that exhibits 80%, 85%, 90%, 91%, 92%, 93%, 94%, 95%, 96%, 97%, 98%, 99%, 100%, or a range between any two of these values, sequence identity to the nucleic acid sequence of any one of SEQ ID NOs: 1 and 46-52. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence of SEQ ID NO: 1 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to any one of the sequences described in FIGS. 11A-B and/or FIG. 1A in the publication by Toribio et al. (2016 supra), the content of which is hereby incorporated by reference in its entirety.

Accordingly, in some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence of any one of SEQ ID NOS: 46-52 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 46 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 47 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 48 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 49 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 50 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 51 disclosed herein. In some embodiments, the nucleic acid molecule of the disclosure includes a viral capsid enhancer having a nucleic acid sequence that exhibits at least 80%, at least 85%, at least 90%, at least 95%, at least 96%, at least 97%, at least 98%, at least 99%, or 100% sequence identity to the sequence set forth at SEQ ID NO: 52 disclosed herein.

In the nucleic acid molecule according to some embodiments of the present disclosure, the one or more RNA stem-loops are operably positioned upstream of the coding sequence for the GOI of the second nucleic acid sequence. In some embodiments, the one or more RNA stem-loops are operably positioned from about 1 to about 50 nucleotides, from about 10 to about 75 nucleotides, from about 30 to about 100 nucleotides, from about 40 to about 150 nucleotides, from about 50 to about 200 nucleotides, from about 60 to about 250 nucleotides, from about 100 to about 300 nucleotides, or from about 150 to about 500 nucleotides upstream of the coding sequence for the GOI. In some embodiments, the one or more RNA stem-loops are operably positioned from about 1, about 2, about 5, about 10, about 15, about 20, about 25, about 30, about 40, about 50, about 60, about 70, about 80, about 90, about 100, about 200, about 300, about 400, about 500, or a range between any two of these values, nucleotides upstream of the coding sequence for the GOI. In some embodiments, the one or more RNA stem-loops are operably positioned immediately upstream of the coding sequence for the GOI.

In some embodiments, the nucleic acid molecule further includes a 5′-unstranslated region (5′-UTR) sequence operably positioned upstream to the first nucleic acid sequence. In some embodiments, the 5′-UTR sequence is operably positioned from about 1 to about 50, from about 10 to about 75, from about 30 to about 100, from about 40 to about 150, from about 50 to about 200, from about 60 to about 250, from about 100 to about 300, or from about 150 to about 500 nucleotides upstream of the first nucleic acid sequence. In some embodiments, the 5′-UTR sequence is operably positioned from about 1, about 2, about 5, about 10, about 15, about 20, about 25, about 30, about 40, about 50, about 60, about 70, about 80, about 90, or 100 nucleotides upstream of the first nucleic acid sequence. In some embodiments, the 5′-UTR sequence is operably positioned immediately upstream of the first nucleic acid sequence.

In some embodiments, the 5′ UTR sequence is operably positioned downstream to the promoter. In some embodiments, the 5′-UTR sequence is operably positioned from about 1 to about 50, from about 10 to about 75, from about 30 to about 100, from about 40 to about 150, from about 50 to about 200, from about 60 to about 250, from about 100 to about 300, or from about 150 to about 500 nucleotides downstream of the promoter sequence. In some embodiments, the 5′ UTR sequence is operably positioned from about 1, about 2, about 5, about 10, about 15, about 20, about 25, about 30, about 40, about 50, about 60, about 70, about 80, about 90, or 100 nucleotides downstream of the promoter sequence. In some embodiments, the 5′ UTR sequence is operably positioned immediately downstream to the promoter sequence. In some embodiments, the 5′ UTR sequence is operably positioned downstream to the promoter and upstream to the first nucleic acid sequence.

In some embodiments, the nucleic acid molecule comprises a 3′ unstranslated region (3′ UTR) sequence operably positioned downstream of the second nucleic acid sequence. In some embodiments, the 3′ UTR sequence is operably positioned from about 1 to about 50 nucleotides, from about 10 to about 75 nucleotides, from about 30 to about 100 nucleotides, from about 40 to about 150 nucleotides, from about 50 to about 200 nucleotides, from about 60 to about 250 nucleotides, from about 100 to about 300 nucleotides, or from about 150 to about 500 nucleotides downstream of the second sequence nucleic acid sequence. In some embodiments, the 3′ UTR sequence is operably positioned from about 1, about 2, about 5, about 10, about 15, about 20, about 25, about 30, about 40, about 50, about 60, about 70, about 80, about 90, about 100, about 200, about 300, about 400, about 500, or a range between any two of these values, nucleotides downstream of the second nucleic acid sequence. In some embodiments, the 3′ UTR sequence is operably positioned immediately downstream of the second nucleic acid sequence.

In some embodiments disclosed herein, the coding sequence for the GOI is transcribed into a messenger RNA (mRNA) or part of an mRNA. As used herein, the term “mRNA” or “messenger RNA” refers to a single stranded RNA molecule that is synthesized during transcription, is complementary to one of the strands of double-stranded DNA, and serves to transmit the genetic information contained in DNA to the ribosomes for protein synthesis. The mRNA may be spliced, partially spliced or unspliced, and may be eukaryotic or prokaryotic mRNA. As discussed above, mRNA molecules according to some embodiments of the disclosure can be produced via de novo synthesis. In some embodiments disclosed herein, the coding sequence for the GOI encodes a polypeptide. In some embodiments, the polypeptide is a therapeutic polypeptide, a prophylactic polypeptide, a diagnostic polypeptide, a nutraceutical polypeptide, an industrial enzyme, a reporter polypeptide, or any combination thereof. In some embodiments, the polypeptide is an antibody, an antigen, an immune modulator, a cytokine, an enzyme, or any combination thereof.

In some embodiments, the nucleic acid molecule of the disclosure further includes a coding sequence for an autoprotease peptide (e.g., autocatalytic self-cleaving peptide), where the coding sequence for the autoprotease is optionally operably linked upstream to the second nucleic acid sequence. Generally, any proteolytic cleavage site known in the art can be incorporated into the nucleic acid molecules of the disclosure and can be, for example, proteolytic cleavage sequences that are cleaved post-production by a protease. Further suitable proteolytic cleavage sites also include proteolytic cleavage sequences that can be cleaved following addition of an external protease. As used herein the term “autoprotease” refers to a “self-cleaving” peptide that possesses autoproteolytic activity and is capable of cleaving itself from a larger polypeptide moiety. First identified in the foot-and-mouth disease virus (FMDV), a member of the picornavirus group, several autoproteases have been subsequently identified such as, for example, “2A like” peptides from equine rhinitis A virus (E2A), porcine teschovirus-1 (P2A) and Thosea asigna virus (T2A), and their activities in proteolytic cleavage have been shown in various ex vitro and in vivo eukaryotic systems. As such, the concept of autoproteases is available to one of skill in the art with many naturally-occurring autoprotease systems have been identified. Well studied autoprotease systems are e.g. viral proteases, developmental proteins (e.g. HetR, Hedgehog proteins), RumA autoprotease domain, UmuD, etc.). Non-limiting examples of autoprotease peptides suitable for the compositions and methods of the present disclosure include the peptide sequences from porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

In some embodiments, the coding sequence for an autoprotease peptide is operably linked downstream to the first nucleic acid sequence and upstream to the second nucleic acid sequence. In some embodiments, the autoprotease peptide comprises, or consists of, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the autoprotease peptide includes a peptide sequence of porcine teschovirus-1 2A (P2A).

One of skill in the art will appreciate that different configurations of the viral capsid enhancer sequence, the sequence encoding the autoprotease peptide, and the sequence encoding the gene of interest can be employed as long as the capsid enhancer sequence enhances expression of the heterologous nucleic acid sequence(s), e.g. a coding sequence for a GOI, as compared with the level seen in the absence of the capsid enhancer sequence. These sequences will typically be configured so that the polypeptide encoded by the gene of interest can be released from the protease and any capsid protein sequence after cleavage by the autoprotease.

A non-limiting list of exemplary combinations of autoprotease peptides described herein (such as P2A, F2A, E2A, T2A, BmCPV2A, and BmIFV2A) with one or more viral capsid enhancer sequences described herein are provided in Tables 1 and 2. Table 1 provides a shorthand name for each viral capsid enhancer (e.g., “CE01”) and a shorthand name for each autoprotease peptide (e.g., “AP01”). Each numbered ‘X’ peptide in Table 2 has a corresponding autoprotease peptide provided in Table 1. Likewise, each numbered ‘Y’ enhancer in Table 2 has a corresponding viral capsid enhancer provided in Table 1. Therefore, each “X:Y” entry in Table 2 provides an example of a combination of a viral capsid enhancer and an autoprotease peptide that can be used in the molecules, compositions, and methods of the present disclosure. For example, the combination designated as “AP01:CE16” in Table 2 provides a combination of viral capsid enhancer derived from Sindbis virus (SINV) and an autoprotease peptide from porcine teschovirus-1 2A (P2A).

TABLE 1
Exemplary viral capsid enhancers and autoprotease peptides of the disclosure

| Viral Capsid Enhancer (Y) | | Autoprotease Peptide (X) | |
|---|---|---|---|
| Eastern equine encephalitis virus (EEEV) | (CE01) | porcine teschovirus-1 2A (P2A) | (AP01) |
| Venezuelan equine encephalitis virus | (CE02) | foot-and-mouth disease virus | (AP02) |
| (VEEV) | | (FMDV) 2A (F2A) | |
| Everglades virus (EVEV) | (CE03) | Equine Rhinitis A Virus (ERAV) | (AP03) |
| | | 2A (E2A) | |
| Mucambo virus (MUCV) | | Thosea asigna virus 2A (T2A) | (AP04) |
| Semliki forest virus (SFV) | (CE04) | cytoplasmic polyhedrosis virus | (AP05) |
| | | 2A (BmCPV2A) | |
| Pixuna virus (PIXV) | (CE05) | Flacherie Virus 2A (BmIFV2A) | (AP06) |
| Middleburg virus (MIDV) | (CE06) | | |
| Chikungunya virus (CHIKV) | (CE07) | | |
| O'Nyong-Nyong virus (ONNV) | (CE08) | | |
| Ross River virus (RRV) | (CE09) | | |
| Barmah Forest virus (BF) | (CE10) | | |
| Getah virus (GET) | (CE11) | | |
| Sagiyama virus (SAGV) | (CE12) | | |
| Bebaru virus (BEBV) | (CE13) | | |
| Mayaro virus (MAYV) | (CE14) | | |
| Una virus (UNAV) | (CE15) | | |
| Sindbis virus (SINV) | (CE16) | | |
| Aura virus (AURAV) | (CE17) | | |
| Whataroa virus (WHAV) | (CE18) | | |
| Babanki virus (BABV) | (CE19) | | |
| Kyzylagach virus (KYZV) | (CE20) | | |
| Western equine encephalitis virus (WEEV) | (CE21) | | |
| Highland J virus (HJV) | (CE22) | | |
| Fort Morgan virus (FMV) | (CE23) | | |
| Ndumu (NDUV) | (CE24) | | |
| Salmonid alphavirus (SAV) | (CE25) | | |
| Buggy Creek virus | (CE26) | | |

TABLE 2
X:Y
AP01:CE01
AP01:CE02
AP01:CE03
AP01:CE04
AP01:CE05
AP01:CE06
AP01:CE07
AP01:CE08
AP01:CE09
AP01:CE10
AP01:CE11
AP01:CE12
AP01:CE13
AP01:CE14
AP01:CE15
AP01:CE16
AP01:CE17
AP01:CE18
AP01:CE19
AP01:CE20
AP01:CE21
AP01:CE22
AP01:CE23
AP01:CE24
AP01:CE25
AP01:CE26
AP02:CE01
AP02:CE02
AP02:CE03
AP02:CE04
AP02:CE05
AP02:CE06
AP02:CE07
AP02:CE08
AP02:CE09
AP02:CE10
AP02:CE11
AP02:CE12
AP02:CE13
AP02:CE14
AP02:CE15
AP02:CE16
AP02:CE17
AP02:CE18
AP02:CE19
AP02:CE20
AP02:CE21
AP02:CE22
AP02:CE23
AP02:CE24
AP02:CE25
AP02:CE26
AP03:CE01
AP03:CE02
AP03:CE03
AP03:CE04
AP03:CE05
AP03:CE06
AP03:CE07
AP03:CE08
AP03:CE09
AP03:CE10
AP03:CE11
AP03:CE12
AP03:CE13
AP03:CE14
AP03:CE15
AP03:CE16
AP03:CE17
AP03:CE18
AP03:CE19
AP03:CE20
AP03:CE21
AP03:CE22
AP03:CE23
AP03:CE24
AP03:CE25
AP03:CE26
AP04:CE01
AP04:CE02
AP04:CE03
AP04:CE04
AP04:CE05
AP04:CE06
AP04:CE07
AP04:CE08
AP04:CE09
AP04:CE10
AP04:CE11
AP04:CE12
AP04:CE13
AP04:CE14
AP04:CE15
AP04:CE16
AP04:CE17
AP04:CE18
AP04:CE19
AP04:CE20
AP04:CE21
AP04:CE22
AP04:CE23
AP04:CE24
AP04:CE25
AP04:CE26
AP05:CE01
AP05:CE02
AP05:CE03
AP05:CE04
AP05:CE05
AP05:CE06
AP05:CE07
AP05:CE08
AP05:CE09
AP05:CE10
AP05:CE11
AP05:CE12
AP05:CE13
AP05:CE14
AP05:CE15
AP05:CE16
AP05:CE17
AP05:CE18
AP05:CE19
AP05:CE20
AP05:CE21
AP05:CE22
AP05:CE23
AP05:CE24
AP05:CE25
AP05:CE26
AP06:CE01
AP06:CE02
AP06:CE03
AP06:CE04
AP06:CE05
AP06:CE06
AP06:CE07
AP06:CE08
AP06:CE09
AP06:CE10
AP06:CE11
AP06:CE12
AP06:CE13
AP06:CE14
AP06:CE15
AP06:CE16
AP06:CE17
AP06:CE18
AP06:CE19
AP06:CE20
AP06:CE21
AP06:CE22
AP06:CE23
AP06:CE24
AP06:CE25
AP06:CE26

In one aspect, disclosed herein are novel nucleic acid molecules which include a nucleic acid sequence encoding a modified viral RNA replicon, wherein the modified viral RNA replicon includes a first nucleic acid sequence encoding one or more structural elements of a viral capsid enhancer (e.g., a DLP motif) or a variant thereof, wherein the viral capsid enhancer is heterologous to the viral RNA replicon, and a second nucleic acid sequence encoding at least one nonstructural viral protein or a portion thereof, wherein the first nucleic acid sequence is operably linked upstream to the second nucleic acid sequence.

The terms “replicon RNA” and “RNA replicon” used interchangeably herein, refers to RNA which contains all of the genetic information required for directing its own amplification or self-replication within a permissive cell. To direct its own replication, the RNA molecule 1) encodes polymerase, replicase, or other proteins which may interact with viral or host cell-derived proteins, nucleic acids or ribonucleoproteins to catalyze the RNA amplification process; and 2) contain cis-acting RNA sequences required for replication and transcription of the subgenomic replicon-encoded RNA. These sequences may be bound during the process of replication to its self-encoded proteins, or non-self-encoded cell-derived proteins, nucleic acids or ribonucleoproteins, or complexes between any of these components. In some embodiments of the present disclosure, a modified viral replicon RNA molecule typically contains the following ordered elements: 5′ viral or defective-interfering RNA sequence(s) required in cis for replication, sequences coding for biologically active nonstructural proteins, promoter for the subgenomic RNA, 3′ viral sequences required in cis for replication, and a polyadenylate tract. Further, the term replicon RNA generally refers to a molecule of positive polarity, or “message” sense, and the replicon RNA may be of length different from that of any known, naturally-occurring RNA viruses. In some embodiments of the present disclosure, the replicon RNA does not contain coding sequences for at least one of the structural viral proteins. In these instances, the sequences encoding structural genes can be substituted with one or more heterologous sequences such as, for example, a coding sequence for a gene of interest (GOI). In those instances where the replicon RNA is to be packaged into a recombinant alphavirus particle, it must contain one or more sequences, so-called packaging signals, which serve to initiate interactions with alphavirus structural proteins that lead to particle formation.

As used herein, “subgenomic RNA” refers to a RNA molecule of a length or size which is smaller than the genomic RNA from which it was derived. The viral subgenomic RNA should be transcribed from an internal promoter, whose sequences reside within the genomic RNA or its complement. Transcription of a subgenomic RNA may be mediated by viral-encoded polymerase(s) associated with host cell-encoded proteins, ribonucleoprotein(s), or a combination thereof. In some embodiments of the present disclosure, the subgenomic RNA is produced from a modified replicon RNA as disclosed herein and encodes or expresses one or more gene of interest (GOI). Instead of the native subgenomic promoter, the subgenomic RNA can be placed under control of internal ribosome entry site (IRES) derived from encephalomyocarditis viruses (EMCV), Bovine Viral Diarrhea Viruses (BVDV), polioviruses, Foot-and-mouth disease viruses (FMD), enterovirus 71, or hepatitis C viruses.

In some embodiments, the second nucleic acid sequence of the modified viral RNA replicon includes the coding sequence for at least one, at least two, at least three, or at least four nonstructural viral proteins. In some embodiments, the second nucleic acid sequence of the modified viral RNA replicon includes the coding sequence for a portion of the at least one nonstructural viral protein. For example, the second nucleic acid sequence of the modified viral RNA replicon can include about 10%, 20%, 30%, 40%, 50%, 60%, 70%, 80%, 90%, 95%, 100%, or a range between any two of these values, of the encoding sequence for the at least one nonstructural viral protein. In some embodiments, the second nucleic acid sequence of the modified viral RNA replicon can include the coding sequence for a substantial portion of the at least one nonstructural viral protein. As used herein, a “substantial portion” of a nucleic acid sequence encoding a nonstructural viral protein comprises enough of the nucleic acid sequence encoding the nonstructural viral protein to afford putative identification of that protein, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). In some embodiments, the second nucleic acid sequence of the modified viral RNA replicon can include the entire coding sequence for the at least one nonstructural protein. In some embodiments, the second nucleic acid sequence comprises substantially all the coding sequence for the native viral nonstructural proteins.

The molecular techniques and methods by which these new nucleic acid molecules were constructed and characterized are described more fully in the Examples herein of the present application. As non-limiting examples, in the Examples section, the Venezuelan equine encephalitis virus (VEEV) and Equine arteritis virus (EAV) have been used to illustrate the compositions and methods disclosed herein.

In some embodiments, the nucleic acid molecules disclosed herein are recombinant nucleic acid molecules. As used herein, the term recombinant means any molecule (e.g. DNA, RNA, etc.), that is, or results, however indirect, from human manipulation of a polynucleotide. As non-limiting examples, a cDNA is a recombinant DNA molecule, as is any nucleic acid molecule that has been generated by ex vitro polymerase reaction(s), or to which linkers have been attached, or that has been integrated into a vector, such as a cloning vector or expression vector. As non-limiting examples, a recombinant nucleic acid molecule: 1) has been synthesized or modified ex vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature; 3) has been engineered using molecular cloning techniques such that it lacks one or more nucleotides with respect to the naturally-occurring nucleic acid sequence; and/or 4) has been manipulated using molecular cloning techniques such that it has one or more sequence changes or rearrangements with respect to the naturally-occurring nucleic acid sequence.

A nucleic acid molecule, including a variant of a naturally-occurring nucleic acid sequence, can be produced using a number of methods known to those skilled in the art. The sequence of a nucleic acid molecule can be modified with respect to a naturally-occurring sequence from which it is derived using a variety of techniques including, but not limited to, classic mutagenesis techniques and recombinant DNA techniques, such as but not limited to site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, recombinational cloning, and chemical synthesis, including chemical synthesis of oligonucleotide mixtures and ligation of mixture groups to “build” a mixture of nucleic acid molecules, and combinations thereof. Nucleic acid molecule homologs can be selected from a mixture of modified nucleic acid molecules by screening for the function of the protein or the replicon encoded by the nucleic acid molecule and/or by hybridization with a wild-type gene or fragment thereof, or by PCR using primers having homology to a target or wild-type nucleic acid molecule or sequence.

In various embodiments disclosed herein, the nucleic acid molecule disclosed herein can include one or more of the following features.

In some embodiments, the modified viral RNA replicon includes a modified RNA replicon derived from a virus species belonging to the Alphavirus genus of the Togaviridae family or to the Arterivirus genus of the Arteriviridae family. Suitable arterivirus species includes Equine arteritis virus (EAV), Porcine respiratory and reproductive syndrome virus (PRRSV), Lactate dehydrogenase elevating virus (LDV), Simian hemorrhagic fever virus (SHFV), and wobbly possum disease virus (WPDV). Virulent and avirulent arterivirus strains are both suitable. Non-limiting examples of preferred arterivirus strains include, but not limited to, EAV-virulent Bucyrus strain (VBS), LDV-Plagemann, LDV-C, PRRSV-type 1, and PRRSV-type 2. Exemplary preferred EAV strains include, but not limited to, EAV VB53, EAV ATCC VR-796, EAV HK25, EAV HK116, EAV ARVAC MLV, EAV Bucyrus strain (Ohio), modified EAV Bucyrus, avirulant strain CA95, Red Mile (Kentucky), 84KY-A1 (Kentucky), Wroclaw-2 (Poland), Bibuna (Switzerland), and Vienna (Australia). Non-limiting preferred examples of PRRSV strains include PRRSV LV4.2.1, PRRSV 16244B, PRRSV HB-1(sh)/2002, PRRSV HB-2(sh)/2002, PRRSV HN1, PRRSV SD 01-08, PRRSV SD0802, PRRSV SD0803, PRRSV, and VR2332. Non-limiting preferred examples of SHFV strains and variants include SHFV variants SHFV-krtgla and -krtglb (SHFV-krtgla/b), SHFVkrtg2a/b (GenBank accession #JX473847 to JX473850), SHFV-LVR, the SHFV prototype variant LVR 42-0/M6941 (NC_003092); SHFV-krcl and SHFVkrc2 from Kibale red colobus (HQ845737 and HQ845738, respectively). Other non-limiting examples of preferred arteriviruses include PRRSV-Lelystad, the European (type 1) type strain (M96262); PRRSVVR2332, the North American (type 2) type strain (U87392); EAV-Bucyrus (NC_002532); EAV-s3685 (GQ903794); LDV-P, the Plagemann strain (U15146); and LDV-C, the neurovirulent type C strain (L13298).

In some embodiments, the first nucleic acid sequence is positioned upstream to a nucleic acid sequence encoding a portion or the entire pp1ab nonstructural protein of the modified arterivirus RNA replicon. In some embodiments, the first nucleic acid sequence is operably positioned within a region of about 1 to 1000 nucleotides downstream of the 5′-terminus of the modified viral RNA replicon. In some embodiments, the first nucleic acid sequence is operably positioned within a region of about 1 to 25, about 1 to 40, about 10 to 25, 10 to 50, about 10 to 100, about 20 to 50, about 20 to 75, about 25 to 100, about 25 to 100 nucleotides downstream of the 5′-terminus of the modified viral RNA replicon. In some embodiments, the first nucleic acid sequence is operably positioned within a region of about 1, 2, 5, 10, 15, 20, 25, 30, 40, 50, 75, 100, 125, 150, 200, 250, 300, or more, or a range between any two of these values, nucleotides downstream of the 5′-terminus of the modified viral RNA replicon. In some embodiments, the first nucleic acid sequence is operably positioned within a region of about 1 to 100, about 1 to 500, about 25 to 800, about 50 to 900, about 50 to 300, about 25 to 200, about 25 to 100, about 50 to 400, about 100 to 500, about 100 to 300, about 100 to 200, about 200 to 500, about 200 to 600, about 200 to 400, about 150 to 700, about 150 to 400, or about 500 to 1000 nucleotides downstream of the 5′-terminus of the modified viral RNA replicon.

Without being bound by any particular theory, it is believed that translational enhancing activity of a viral DLP motif can depend, in some embodiments, on the distance between the viral DLP motif and the initiation AUGi codon (Toribio et al., 2016 supra). Accordingly, in some embodiments, the first nucleic acid sequence is operably positioned a region of about 10 to 100 nucleotides downstream of the initiation codon AUGi of the modified viral RNA replicon. In some embodiments, the first nucleic acid sequence is operably positioned within a region of about 10 to 75, about 10 to 50, about 10 to 25, 15 to 75, about 15 to 50, about 15 to 25, about 25 to 75, about 25 to 50, about 25 to 100 nucleotides downstream of the initiation codon AUGi of the modified viral RNA replicon. In some embodiments, the first nucleic acid sequence is operably positioned within a region of about 25, 28, 31, 34, 37, 37, 40, 43, 46, 49, 50, or a range between any two of these values, nucleotides downstream of the initiation codon AUGi of the modified viral RNA replicon.

In some embodiments, the sequence encoding the modified viral RNA replicon further comprising one or more expression cassettes, wherein each of the expression cassettes comprises a promoter operably linked to a coding sequence for a gene of interest (GOI). As used herein, the term “expression cassette” refers to a construct of genetic material that contains coding sequences and enough regulatory information to direct proper transcription and/or translation of the coding sequences in a recipient cell, in vivo and/or ex vivo. The expression cassette may be inserted into a vector for targeting to a desired host cell and/or into a subject. Further, the term expression cassette may be used interchangeably with the term “expression construct”. The term “expression cassette” as used herein, refers to a nucleic acid construct that encodes a protein or functional RNA operably linked to expression control elements, such as a promoter, and optionally, any or a combination of other nucleic acid sequences that affect the transcription or translation of the gene.

The term “operably linked”, as used herein, denotes a functional linkage between two or more sequences. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous.

The basic techniques for operably linking two or more sequences of DNA together are familiar to one of ordinary skill in the art, and such methods have been described in many books for standard molecular biological manipulation (see, for example, Maniatis et al., “Molecular Cloning: A Laboratory Manual” 2nd ed. Cold Spring Harbor Laboratory Press, Cold Spring Harbor, N.Y.; and Gibson et al., Nature Methods 6:343-45, 2009).

In some embodiments disclosed herein, the nucleic acid molecules disclosed herein can include more than one expression cassette. In principle, the nucleic acid molecules disclosed herein can generally include any number of expression cassettes. In some particular embodiments, the modified viral RNA replicon comprises at least two, three, four, five, or six expression cassettes. In some embodiments, at least one of the one or more expression cassettes is operably positioned downstream to a transcriptional regulatory sequence (TRS) of the modified arterivirus RNA replicon, wherein the TRS can be TRS1, TRS2, TRS3, TRS4, TRS5, TRS6, TRS7, or a combination thereof. In some particular embodiments, at least one of the one or more expression cassettes is operably positioned downstream of the TRS7 of the modified arterivirus RNA replicon.

The nucleic acid molecules as provided herein can find use, for example, as an expression or transcription vector that, when operably linked to a heterologous nucleic acid sequence such as, for example, a coding sequence of a gene of interest (GOI), can affect expression of the GOI. In some embodiments, the coding sequence of the GOI is optimized for expression at a level higher than the expression level of a reference coding sequence. In some embodiments, the reference coding sequence is a not codon-optimized. In some embodiments, the GOI coding sequence comprises codon optimization. With respect to codon-optimization of nucleic acid sequences, degeneracy of the genetic code provides the possibility to substitute at least one base of the protein encoding sequence of a gene with a different base without causing the amino acid sequence of the polypeptide produced from the gene to be changed. Hence, the nucleic acid molecules of the present disclosure may also have one or more nucleotide substitutions in accordance with degeneracy of the genetic code. References describing codon usage are readily publicly available. In some further embodiments of the disclosure, polynucleotide sequence variants can be produced for a variety of reasons, e.g., to optimize codon expression for a particular host (e.g., changing codons in the arterivirus mRNA to those preferred by other organisms such as human, hamster, mice, or monkey).

In some embodiments disclosed herein, the sequence of the GOI encode a polypeptide. The type of the polypeptide can vary depending on specific applications. For example, the polypeptide can be a therapeutic polypeptide, a prophylactic polypeptide, a diagnostic polypeptide, a nutraceutical polypeptide, an industrial enzyme, a reporter polypeptide, or any combination thereof. In some embodiments, the polypeptide is an antibody, an antigen, an immune modulator, a cytokine, an enzyme, or a combination thereof.

In some embodiments, the nucleic acid molecule as disclosed herein can further comprise a third nucleic acid sequence encoding one or more structural elements of a second viral capsid enhancer (e.g., a DLP motif), wherein the third nucleic acid sequence is operably linked upstream to the coding sequence for the GOI. The second DLP motif may be the same or may be different from the first DLP motif positioned upstream of the coding sequence for the nonstructural proteins. Accordingly, in some embodiments, the second DLP motif is the same as the first DLP motif positioned upstream of the coding sequence for the nonstructural proteins. In some embodiments, the second DLP motif is different from the first DLP motif positioned upstream of the coding sequence for the nonstructural proteins.

In some embodiments, the sequence encoding the modified viral RNA replicon further comprising a coding sequence for a proteolytic cleavage site operably linked downstream to the third nucleic acid sequence and upstream to the coding sequence for the GOI. Generally, any proteolytic cleavage site known in the art can be incorporated into the nucleic acid molecules of the disclosure and can be, for example, proteolytic cleavage sequences that are cleaved post-production by a protease. Further suitable proteolytic cleavage sites also include proteolytic cleavage sequences that can be cleaved following addition of an external protease. In some embodiments, the sequence encoding the modified viral RNA replicon further comprising a coding sequence for an autoprotease peptide operably linked downstream to the third nucleic acid sequence and upstream to the coding sequence for the GOI. In some embodiments, the autoprotease peptide includes a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the autoprotease peptide includes a peptide sequence from porcine teschovirus-1 2A (P2A).

One of skill in the art will appreciate that different configurations of the viral capsid enhancer sequence, the coding sequence for the nonstructural proteins, the sequence encoding the autoprotease peptide, and the sequence encoding the gene of interest can be employed as long as the capsid enhancer sequence augments expression of the heterologous nucleic acid sequence(s), as compared with the level seen in the absence of the capsid enhancer sequence. These sequences will typically be configured so that the polypeptide encoded by the gene of interest can be released from the protease and any capsid protein sequence after cleavage by the autoprotease.

In some embodiments, the sequence of the nucleic acid molecule as disclosed herein includes a modified RNA replicon of an alphavirus virus species. In some embodiments, the modified alphavirus RNA replicon is of an alphavirus belonging to the VEEV/EEEV group, or the SF group, or the SIN group. Non-limiting examples of SF group alphaviruses include Semliki Forest virus, O'Nyong-Nyong virus, Ross River virus, Middelburg virus, Chikungunya virus, Barmah Forest virus, Getah virus, Mayaro virus, Sagiyama virus, Bebaru virus, and Una virus. Non-limiting examples of SIN group alphaviruses include Sindbis virus, Girdwood S.A. virus, South African Arbovirus No. 86, Ockelbo virus, Aura virus, Babanki virus, Whataroa virus, and Kyzylagach virus. Non-limiting examples of VEEV/EEEV group alphaviruses include Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), and Una virus (UNAV).

Non-limiting examples of alphavirus species include Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus. Virulent and avirulent alphavirus strains are both suitable. In some embodiments, the modified alphavirus RNA replicon is of a Sindbis virus (SIN), a Semliki Forest virus (SFV), a Ross River virus (RRV), a Venezuelan equine encephalitis virus (VEEV), or an Eastern equine encephalitis virus (EEEV). In some embodiments, the modified alphavirus RNA replicon is of a Venezuelan equine encephalitis virus (VEEV).

In some instances where the nucleic acid molecule as disclosed herein includes a modified RNA replicon of an alphavirus virus species, the first nucleic acid sequence is positioned upstream to a nucleic acid sequence encoding one or more nonstructural proteins nsp1-4 or a portion thereof of the modified alphavirus RNA replicon. Accordingly, in some embodiments, the first nucleic acid sequence is positioned upstream to a nucleic acid sequence encoding the nonstructural proteins nsp1, nsp1-2, nsp1-3, nsp1-4, nsp2-4, nsp3-4, nsp2-3, nsp2, nsp3, nsp4, or a portion thereof of the modified alphavirus RNA replicon. In some embodiments, the sequence encoding the modified alphavirus RNA replicon further includes one or more expression cassettes, wherein each of the expression cassettes includes a promoter operably linked to a coding sequence for a gene of interest (GOI). In some embodiments, the modified alphavirus RNA replicon comprises at least two, three, four, five, or six expression cassettes. In some embodiments, at least one of the one or more expression cassettes is operably linked downstream of a nucleic acid sequence encoding one or more nonstructural proteins nsp1-4 or a portion thereof of the modified alphavirus RNA replicon. Accordingly, in some embodiments, at least one of the one or more expression cassettes is operably linked downstream of a nucleic acid sequence encoding the nonstructural proteins nsp1, nsp1-2, nsp1-3, nsp1-4, nsp2-4, nsp3-4, nsp2-3, nsp2, nsp3, nsp4, or a portion thereof, of the modified alphavirus RNA replicon.

In some embodiments, at least one of the one or more expression cassettes further comprises a third nucleic acid sequence encoding one or more structural elements of a second viral capsid enhancer (e.g., a DLP motif), wherein the third nucleic acid sequence is operably linked upstream to the coding sequence for the GOI. The second DLP motif may be the same or may be different from the first DLP motif positioned upstream of the coding sequence for at least of the nonstructural proteins nsp1-4 or a portion thereof. Accordingly, in some embodiments, the second DLP motif is the same as the first DLP motif positioned upstream of the coding sequence for the nonstructural proteins. In some embodiments, the second DLP motif is different from the first DLP motif positioned upstream of the coding sequence for the nonstructural proteins.

In some embodiments, the nucleic acid sequence of the present disclosure further comprises a coding sequence for an autoprotease peptide operably linked downstream to the third nucleic acid sequence and upstream to the coding sequence for the GOI. The autoprotease peptide can generally be any autoprotease peptide known in the art. Non-limiting examples of auprotease peptides include the peptide sequences from porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and any combinations thereof.

In a further aspect, some embodiments disclosed herein relate to a nucleic acid molecule including a nucleic acid sequence encoding a modified non-alphavirus RNA replicon, wherein the modified non-alphavirus RNA replicon comprising a first nucleic acid sequence encoding a viral capsid enhancer (e.g., a DLP motif). In some embodiments, the modified non-alphavirus RNA replicon further comprising a second nucleic acid sequence encoding at least one nonstructural viral protein or a portion thereof, wherein the first nucleic acid sequence is operably linked upstream to the second nucleic acid sequence.

In some embodiments, the modified non-alphavirus RNA replicon further comprising a coding sequence for an autoprotease peptide operably linked downstream to the first nucleic acid sequence and upstream to the second nucleic acid sequence. In some embodiments, the modified non-alphavirus RNA replicon includes a modified RNA replicon of a positive-strand RNA virus. In some embodiments, the modified non-alphavirus RNA replicon includes a modified RNA replicon of a negative-strand RNA virus.

Non-limiting examples of modified non-alphavirus RNA replicons include modified RNA replicons of virus species belonging to Togaviridae family, Flaviviridae family, Orthomyxoviridae family, Rhabdoviridae family, or Paramyxoviridae family. Accordingly, in some embodiments, the modified non-alphavirus RNA replicon includes a modified RNA replicon of a negative-strand RNA virus. Suitable negative-strand RNA virus species include, but are not limited to viral species of the families Orthomyxoviridae, Rhabdoviridae, and Paramyxoviridae. In some embodiments, the modified non-alphavirus RNA replicon includes a modified RNA replicon of a positive-strand virus species belonging to the Togaviridae family or Flaviviridae family. In some embodiments, the modified non-alphavirus RNA replicon includes a modified RNA replicon of a positive-strand virus species belonging to the Arterivirus genus of the Arteriviridae family. Suitable arterivirus species include, but are not limited to, species of Equine arteritis virus (EAV), Porcine respiratory and reproductive syndrome virus (PRRSV), Lactate dehydrogenase elevating virus (LDV), Simian hemorrhagic fever virus (SHFV), and wobbly possum disease virus (WPDV).

In some embodiments, the sequence encoding the non-alphavirus modified RNA replicon further includes one or more expression cassettes, wherein each of the expression cassettes comprises a promoter operably linked to a coding sequence for a gene of interest (GOI). In some embodiments, the modified non-alphavirus RNA replicon comprises at least two, three, four, five, or six expression cassettes. In some embodiments, at least one of the one or more expression cassettes is operably linked downstream of the second nucleic acid sequence encoding the at least one nonstructural viral protein or a portion thereof. In some embodiments, at least one of the one or more expression cassettes further comprises a third nucleic acid sequence encoding one or more structural elements of a viral capsid enhancer, wherein the third nucleic acid sequence is operably linked upstream to the coding sequence for the GOI. In some embodiments, the modified non-alphavirus RNA replicon further includes a coding sequence for an autoprotease peptide operably linked downstream to the third nucleic acid sequence and upstream to the coding sequence for the GOI.

Some embodiments of the disclosure relate to a nucleic acid molecule including a nucleic acid sequence encoding a modified viral RNA replicon which includes in 5′->3′direction a first nucleic acid sequence encoding a capsid enhancer from a Sindbis virus, a second nucleic acid sequence encoding an autoprotease peptide, and a third nucleic acid sequence encoding all of the viral nonstructural proteins. Some embodiments of the disclosure relate to a nucleic acid molecule including a nucleic acid sequence which encodes a modified viral RNA replicon, wherein the modified viral RNA replicon comprises a viral capsid enhancer and wherein the sequence of the modified viral RNA replicon exhibits at least 80% sequence identity to the sequence of at least one of SEQ ID NOs: 15-18 and 27-29.

Contemplated within the scope of the present disclosure are variants of the polynucleotides provided herein. Such variants may be naturally-occurring, including homologous polynucleotides from the same or a different species, or may be non-natural variants, for example polynucleotides synthesized using chemical synthesis methods, or generated using recombinant DNA techniques. With respect to nucleic acid sequences, degeneracy of the genetic code provides the possibility to substitute at least one base of the protein encoding sequence of a gene with a different base without causing the amino acid sequence of the polypeptide produced from the gene to be changed. Hence, the nucleic acid molecules of the present disclosure may also have any base sequence that has been changed from any polynucleotide sequence disclosed herein by substitution in accordance with degeneracy of the genetic code. References describing codon usage are readily publicly available. In further embodiments, polynucleotide sequence variants can be produced for a variety of reasons, e.g., to optimize codon expression for a particular host (e.g., changing codons in the viral mRNA to those preferred by other organisms such as mammals or fish species).

In some embodiments, the nucleic acid molecules of the present disclosure comprises in 5′->3′direction a nucleic acid sequence encoding a capsid enhancer from a Sindbis virus, a nucleic acid sequence encoding an autoprotease peptide, and a nucleic acid sequence encoding all of the viral nonstructural proteins of a modified viral RNA replicon. In some embodiments, the nucleic acid molecule comprises in 5′->3′direction a 5-UTR sequence, a first capsid enhancer from a Sindbis virus, an autoprotease peptide, a sequence encoding all of the viral nonstructural proteins of a modified viral RNA replicon, one or more expression cassettes, and a 3′UTR sequence, wherein at least one of the one or more expression cassettes comprises a second capsid enhancer from a Sindbis virus operably linked upstream of a coding sequence for a gene of interest (GOI).

Accordingly, in some embodiments, the nucleic acid molecule of the present disclosure includes a nucleic acid sequence which encodes a modified viral RNA replicon, wherein the sequence exhibits at least 90%, at least 95%, at least 96%, at least 97%, at least 98%, at least 99%, or 100% sequence identity to the sequence of at least one of SEQ ID NOs: 15-18 and 27-29.

In some embodiments, the nucleic acid molecule of the disclosure is an expression vector. In some embodiments, the expression vector further includes one or more additional regulatory sequences, which can be a transcriptional regulatory element or a translational regulatory element. The terms “regulatory element” and “regulatory region”, as used interchangeably in the present disclosure, refer to a nucleic acid sequence that influences transcription or translation initiation and rate, and stability and/or mobility of a transcription or translation product. Such regulatory elements need not be of naturally-occurring sequences. Regulatory sequences include but are not limited to promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. In some embodiments, the expression vector of the disclosure further includes one or more of the following: an origin of replication, one or more sequences for promoting integration of the expression cassette into the host genome, a terminator sequence.

In some embodiments, the expression vector comprises at least one origin of replication (“ORI”) sequence for replication in a cell. The vectors may further optionally comprise one or more selectable markers under the control of one or more eukaryotic promoters, one or more selectable markers under the control of one or more prokaryotic promoters, and/or one or more sequences that mediate recombination of an exogenous nucleic acid sequence into the target cell's genome.

An ORI is the sequence in a DNA molecule at which replication begins. The ORI serves as a base of assembly for the pre-replication complex. Depending on the ORI, such replication can proceed uni-directionally or bi-directionally. An expression vector as provided herein can include an ORI for replication of the expression vector in a cloning host, such as E. coli or yeast, and/or can include an ORI for replication of the expression vector in a target cell, which can be, for example, a mammalian cell. The structural biology of ORIs is widely conserved among prokaryotes, eukaryotes, and viruses. Most ORIs possess simple tri-, tetra-, or higher nucleotide repetition patterns. Most are AT-rich and contain inverted repeats. Those skilled in the art will be familiar with the more common ORIs, such as P15A and the pUC's ORI.

The expression vector can also, in some embodiments, carry a selectable marker. By way of example, a vector that includes an expression cassette may include, as a selectable marker, a gene conferring resistance to a poisonous substance, such as an antibiotic, a herbicide, or some other toxin, so that transformants can be selected by exposing the cells to the poison and selecting those cells which survive the encounter. In some embodiments, the selectable marker may be under the control of a promoter. In some embodiments, the promoter regulating expression of the selectable marker may be conditional or inducible. In some embodiments, the promoter regulating expression of the selectable marker may be preferably constitutive, and can be, for example, any promoter described herein or another promoter.

In some embodiments, the expression vector is a plasmid, a bacteriophage vector, a cosmid, a fosmid, a viral replicon, a shuttle vector, or a combination thereof. In some embodiments, the expression vector is an RNA replicon. In some embodiments, the expression vector is a prokaryotic expression vector. In some embodiments, the expression vector is a eukaryotic expression vector. In some embodiments, the nucleic acid molecule of the disclosure is produced via de novo synthesis. In some embodiments of the disclosure, de novo synthesis can be used to generate a synthetic mRNA molecule.

### Recombinant Cells

In one aspect, some embodiments disclosed herein relate to a method of transforming a cell that includes introducing into a host cell, such as an animal cell, a nucleic acid molecule as provided herein, and selecting or screening for a transformed cell. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. In some embodiments, the nucleic acid molecule is introduced into a host cell by an electroporation procedure or a biolistic procedure.

In a related aspect, some embodiments relate to recombinant host cells, for example, recombinant animal cells that include a nucleic acid molecule described herein. The nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas9, or DNA-guided endonuclease genome editing NgAgo (Natronobacterium gregoryi Argonaute), or TALEN genome editing (transcription activator-like effector nucleases). In some embodiments, the nucleic acid molecule present in the recombinant host cell as a mini-circle expression vector for a stable or transient expression.

In some embodiments, host cells can be genetically engineered (e.g. transduced or transformed or transfected) with, for example, a vector construct of the present application that can be, for example, a vector for homologous recombination that includes nucleic acid sequences homologous to a portion of the genome of the host cell, or can be an expression vector for the expression of any or a combination of the genes of interest. The vector can be, for example, in the form of a plasmid, a viral particle, a phage, etc. In some embodiments, a vector for expression of a polypeptide of interest can also be designed for integration into the host, e.g., by homologous recombination. The vector containing a polynucleotide sequence as described herein, e.g., nucleic acid molecule comprising a modified alphavirus genome or replicon RNA, as well as, optionally, a selectable marker or reporter gene, can be employed to transform an appropriate host cell.

The methods and compositions disclosed herein may be deployed for genetic engineering of any species, including, but not limited to, prokaryotic and eukaryotic species. Suitable host cells to be modified using the compositions and methods according to the present disclosure can include, but not limited to, algal cells, bacterial cells, heterokonts, fungal cells, chytrid cells, microfungi, microalgae, and animal cells. In some embodiments, the animal cells are invertebrate animal cells. In some embodiments, the vertebrate animal cells are mammalians cells. Host cells can be either untransformed cells or cells that have already been transfected with at least one nucleic acid molecule.

The methods and compositions disclosed herein can be used, for example, with subject and/or host cells that are important or interesting for aquaculture, agriculture, animal husbandry, and/or for therapeutic and medical applications, including production of polypeptides used in the manufacturing of vaccines, pharmaceutical products, industrial products, chemicals, and the like. In some embodiments, the compositions and methods disclosed herein can be used with host cells from species that are natural hosts of alphaviruses, such as rodents, mice, fish, birds, and larger mammals such as humans, horses, pig, monkey, and apes as well as invertebrates. Particularly preferred species, in some embodiments of the application, are vertebrate animal species and invertebrate animal species. In principle, any animal species can be generally used and can be, for example, human, dog, bird, fish, horse, pig, primate, mouse, cotton rat, ferret, cattle, swine, sheep, rabbit, cat, goat, donkey, hamster, or buffalo. Non-limiting examples of suitable bird species include chicken, duck, goose, turkey, ostrich, emu, swan, peafowl, pheasant, partridge, and guinea fowl. In some particular embodiments, the fish is any species in the Salmonidae family. Primary mammalian cells and continuous/immortalized cells types are also suitable. Non-limiting examples of suitable animal host cells include, but not limited to, pulmonary equine artery endothelial cell, equine dermis cell, baby hamster kidney (BHK) cell, rabbit kidney cell, mouse muscle cell, mouse connective tissue cell, human cervix cell, human epidermoid larynx cell, Chinese hamster ovary cell (CHO), human HEK-293 cell, mouse 3T3 cell, Vero cell, Madin-Darby Canine Kidney Epithelial Cell (MDCK), primary chicken fibroblast cell, a HuT78 cell, A549 lung cell, HeLa cell, PER.C6® cell, WI-38 cell, MRC-5 cell, FRhL-2, and CEM T-cell. In some embodiments, the host cell is baby hamster kidney cell. In some embodiments, the baby hamster kidney cell is a BHK-21 cell.

Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Heterologous Nucleic Acid Sequences

In accordance of some embodiments of the present disclosure, a wide variety of nucleic acid sequences can be carried by the nucleic acid molecules of the present disclosure. In some embodiments, nucleic acid molecules as described herein does not contain any additional heterologous nucleic acid sequence. In some embodiments, the nucleic acid molecules of the present disclosure contains one or more additional heterologous or foreign nucleic acid sequences. In some embodiments, the one or more additional heterologous or foreign nucleic acid sequences include a coding sequence for a gene of interest (GOI). In some embodiments disclosed herein, the coding sequence for the GOI encodes a polypeptide or a functional RNA. In some embodiments, the coding sequence for the GOI encodes a functional RNA selected from a ribosomal RNA, a tRNA, a ribozyme, a transactivating (tr) RNA of a CRISPR system, a crispr (cr) RNA of a CRISPR system, a chimeric guide RNA of a CRISPR system, a micro RNA, an interfering RNA (RNAi) molecule, a short hairpin (sh) RNA, or an antisense RNA molecule. In some embodiments, the coding sequence for the GOI encodes a polypeptide selected from the group consisting of a therapeutic polypeptide, a prophylactic polypeptide, a diagnostic polypeptide, a nutraceutical polypeptide, an industrial enzyme, a reporter polypeptide, or any combination thereof. In some embodiments, the coding sequence for the GOI encodes a polypeptide is selected from the group consisting of an antibody, an antigen, an immune modulator, and a cytokine.

In some embodiments, the heterologous nucleic acid sequence comprises a heterologous nucleic acid sequence of at least about 100 bases, 2 kb, 3.5 kb, 5 kb, 7 kb, or 8 kb. The heterologous RNA or heterologous nucleic acid sequence can be chosen from a wide variety of sequences derived from viruses, prokaryotes or eukaryotes. Examples of categories of heterologous sequences include, but are not limited to, immunogens (including native, modified or synthetic antigenic proteins, peptides, epitopes or immunogenic fragments), cytokines, toxins, therapeutic proteins, enzymes, antisense sequences, and immune response modulators.

A wide variety of GOI can be included in the nucleic acid molecules of the present disclosure to express a polypeptide of the GOI, including but not limited to, cytokines, toxins, prodrugs, antigens which stimulate an immune response, ribozymes, and proteins which assist or inhibit an immune response, as well as antisense sequences (or sense sequences for “antisense applications”). As noted above, within various embodiments of the disclosure the modified RNA replicon provided herein may contain the coding region of (and express, in some embodiments) two or more polypeptides of interest.

### 1) Cytokines

In some embodiments disclosed herein, the GOI encodes a cytokine. Generally, cytokines act to proliferate, activate, and/or differentiate immune effectors cells. Examples of cytokines include, but are not limited to macrophages, B lymphocytes, T lymphocytes, endothelial cells, fibroblasts, lymphokines likes gamma interferon, tumor necrosis factor, interleukin, IL-1, IL-2, IL-3, IL-4, IL-5, IL-6, IL-7, IL-8, IL-9, IL-10, IL-1, IL-12, IL-13, IL-14, IL-15, GM-CSF, CSF-1 and G-CSF.

In some related embodiments, the GOI encodes an immunomodulatory cofactor. As utilized within the context of the present disclosure, “immunomodulatory cofactor” refers to factors which, when manufactured by one or more of the cells involved in an immune response, or when added exogenously to the cells, cause the immune response to be different in quality or potency from that which would have occurred in the absence of the cofactor. The quality or potency of a response may be measured by a variety of assays known to one of skill in the art including, for example, ex vitro assays which measure cellular proliferation (e.g., 3H thymidine uptake), and ex vitro cytotoxic assays (e.g., which measure 51 Cr release) (see Warner et al., AIDS Res. and Human Retroviruses 7:645-655, 1991).

Examples of immunomodulatory co-factors include, but are not limited, alpha interferon, gamma interferons, G-CSF, GM-CSF, TNFs, Interleukin-2 (IL-2), IL-4, IL-6, IL-12, IL-15, ICAM-1, ICAM-2, LFA-1, LFA-3, MHC class I molecules, MHC class II molecules, 2-microglobulin, chaperones, CD3, B7/BB 1, MHC linked transporter proteins, and analogues thereof.

The choice of which immunomodulatory cofactor to include within the nucleic acid molecules of the present disclosure may be based upon known therapeutic effects of the cofactor, or experimentally determined. For example, in chronic hepatitis B infections alpha interferon has been found to be efficacious in compensating a patient's immunological deficit and thereby assisting recovery from the disease. In some situations, a suitable immunomodulatory cofactor may be experimentally determined. Briefly, blood samples are first taken from patients with a hepatic disease. Peripheral blood lymphocytes (PBLs) are restimulated ex vitro with autologous or HLA-matched cells (e.g., EBV transformed cells), and transduced with modified arterivirus genome or replicon RNA of the present disclosure which directs the expression of an immunogenic portion of a hepatitis antigen and the immunomodulatory cofactor. Stimulated PBLs are used as effectors in a CTL assay with the BLA-matched transduced cells as targets. An increase in CTL response over that seen in the same assay performed using HLA-matched stimulator and target cells transduced with a vector encoding the antigen alone, indicates a useful immunomodulatory cofactor. In some embodiments, the immunomodulatory cofactor gamma interferon is particularly preferred.

Another non-limiting example of an immunomodulatory cofactor is the B7/BB1 costimulatory factor. Activation of the full functional activity of T cells requires two signals. One signal is provided by interaction of the antigen-specific T cell receptor with peptides which are bound to major histocompatibility complex (MHC) molecules, and the second signal, referred to as costimulation, is delivered to the T cell by antigen-presenting cells. The second signal is required for interleukin-2 (IL-2) production by T cells and appears to involve interaction of the B7/BB 1 molecule on antigen-presenting cells with CD28 and CTLA-4 receptors on T lymphocytes. In some embodiments, B7/BB 1 may be introduced into tumor cells in order to cause costimulation of CD8+ T cells, such that the CD8+ T cells produce enough IL-2 to expand and become fully activated. These CD8+ T cells can kill tumor cells that are not expressing B7 because costimulation is no longer required for further CTL function. Vectors that express both the costimulatory B7/BB1 factor and, for example, an immunogenic HBV core protein, may be constructed utilizing methods which are described herein. Cells transduced with these vectors will become more effective antigen-presenting cells. The HBV core-specific CTL response will be augmented from the fully activated CD8+ T cell via the costimulatory ligand B7/BB 1.

### 2) Toxins

In some embodiments disclosed herein, the GOI encodes a toxin. In some embodiments, toxins act to directly inhibit the growth of a cell. Examples of toxins include, but are not limited to, ricin, abrin, diphtheria toxin, cholera toxin, gelonin, pokeweed, antiviral protein, tritin, Shigella toxin, Pseudomonas exotoxin A, herpes simplex virus thymidine kinase (HSVTK), and E. coli. guanine phosphoribosyl transferase.

### 3) Pro-Drugs

In some embodiments disclosed herein, the GOI encodes a “pro-drug”. As utilized within the context of the present disclosure, “pro-drug” refers to a gene product that activates a compound with little or no cytotoxicity into a toxic product. Representative examples of such gene products include HSVTK and VZVTK (as well as analogues and derivatives thereof), which selectively monophosphorylate certain purine arabinosides and substituted pyrimidine compounds, converting them to cytotoxic or cytostatic metabolites. More specifically, exposure of the drugs ganciclovir, acyclovir, or any of their analogues (e.g., FIAU, FIAC, and DHPG) to HSVTK phosphorylates the drug into its corresponding active nucleotide triphosphate form.

Non-limiting examples of pro-drugs which may be utilized within the context of the present disclosure include: E. coli guanine phosphoribosyl transferase which converts thioxanthine into toxic thioxanthine monophosphate; alkaline phosphatase, which will convert inactive phosphorylated compounds such as mitomycin phosphate and doxorubicin-phosphate to toxic dephosphorylated compounds; fungal (e.g., Fusarium oxysporum) and bacterial cytosine deaminase, which can convert 5-fluorocytosine to the toxic compound 5-fluorouracil; carboxypeptidase G2, which will cleave the glutamic acid from para-N-bis (2-chloroethyl) aminobenzoyl glutamic acid, thereby creating a toxic benzoic acid mustard; and Penicillin-V amidase, which will convert phenoxyacetabide derivatives of doxorubicin and melphalan to toxic compounds.

### 4) Antisense Sequence

In some embodiments disclosed herein, the coding sequence for the GOI is an antisense sequence. Antisense sequences are designed to bind to RNA transcripts, and thereby prevent cellular synthesis of a particular protein or prevent use of that RNA sequence by the cell. Non-limiting examples of such sequences include antisense thymidine kinase, antisense dihydrofolate reductase, antisense HER2, antisense ABL, antisense Myc, antisense ras, as well as antisense sequences which block any of the enzymes in the nucleotide biosynthetic pathway. In addition, in accordance with some embodiments disclosed herein, antisense sequences to interferon and 2 microglobulin may be utilized in order to decrease immune response.

In some embodiments, antisense RNA may be utilized as an anti-tumor agent in order to induce a potent Class I restricted response. In addition to binding RNA and thereby preventing translation of a specific mRNA, high levels of specific antisense sequences are believed to induce the increased expression of interferons (including gamma-interferon) due to the formation of large quantities of double-stranded RNA. The increased expression of gamma interferon, in turn, boosts the expression of MHC Class I antigens. Preferred antisense sequences for use in this regard include actin RNA, myosin RNA, and histone RNA. Antisense RNA which forms a mismatch with actin RNA is particularly preferred.

### 5) Ribozymes

In some embodiments disclosed herein, nucleic acid molecules comprising one or more RNA stem-loop structures are provided which produce ribozymes upon infection of a host cell. Ribozymes are used to cleave specific RNAs and are designed such that it can only affect one specific RNA sequence. Generally, the substrate binding sequence of a ribozyme is between 10 and 20 nucleotides long. The length of this sequence is sufficient to allow a hybridization with target RNA and disassociation of the ribozyme from the cleaved RNA. Representative examples for creating ribozymes include those described in U.S. Pat. Nos. 5,116,742; 5,225,337 and 5,246,921.

### 6) Proteins and Other Cellular Constituents

In some embodiments disclosed herein, a wide variety of proteins or other cellular constituents can be carried by the nucleic acid molecules of the present disclosure. Non-limiting examples of such proteins include native or altered cellular components, as well as foreign proteins or cellular constituents, found in for example, viruses, bacteria, parasites, fungus or animal such as mammalian.

### Methods for Producing Polypeptides

The host cells of the present disclosure, such as a prokaryotic or eukaryotic host cell, can be used to produce (e.g., express) a molecule of interest such as, e.g., a polypeptide, encoded in an open reading frame of a gene of interest (GOI) as disclosed herein. Thus, the present application further provides methods for producing a molecule of interest such as, e.g., a polypeptide, using the host cells and/or the nucleic acid molecules of the present disclosure. The host cells can be, for example, isolated cells, cells in cell culture, cells in a living body, or a combination thereof.

Some embodiments disclosed herein provides methods for producing a polypeptide of interest. The method can include the introduction of a nucleic acid molecule according to any one of the aspects and embodiments of the present disclosure into a host cell, thereby producing a polypeptide encoded by the GOI in the host cell. In some embodiments where the introduced nucleic acid molecule is a RNA molecule, for example an mRNA molecule or a RNA replicon. The RNA molecule can be generated by any method known in the art, for example by de novo synthesis in whole or in part. For example, the RNA molecules, including but not limited to mRNA molecules and RNA replicons, can be produced using chemical methods, enzymatic techniques, or any combination thereof, for example, by chemical synthesis through de novo assembly (such as with oligonucleotides) or in vitro transcription reactions (using appropriate enzymes, buffers, nucleotides, etc.). In some instances where the introduced nucleic acid molecule is an mRNA, the mRNA can be directly delivered to cells in vivo for producing a polypeptide of interest (e.g., drug, antigen, etc.) in cells. The cells can be isolated cells; cells in cell cultures; cells in an tissue, an organ, and/or a subject; or any combination thereof. In some embodiments, no new mRNA copies are made in the cells, As disclosed herein, the incorporation of one or more RNA stem-loops from a viral capsid enhancer (e.g., DLP motifs) into the chemically synthesized RNA can confer the intended enhancement of gene expression once the DLP-containing mRNA is introduced into the cells.

In some embodiments where the introduced nucleic acid molecule is a vector such as, for example, an RNA replicon, new mRNA copies may be generated which includes coding sequence for a gene of interest operably linked to one or more DLP motifs. The incorporation the one or more DLP motifs into the vector, e.g., RNA replicon, can then confer the intended enhancement of gene expression once the DLP-containing vector or replicon is introduced into the cells

Some embodiments disclosed herein provides methods for producing a polypeptide of interest in a host cell. Such method includes the cultivation of a recombinant host cell, including a nucleic acid molecule according to any one of the aspects and embodiments of the present disclosure. In some embodiments, the methods include culturing the host cell of present disclosure (into which a recombinant expression vector encoding the molecule of interest has been introduced) in a suitable medium such that the molecule of interest is produced. In some embodiments, the methods further include isolating the molecule of interest from the medium or the host cell.

Also disclosed are methods for producing a polypeptide of interest in a subject, including administering to the subject a nucleic acid molecule according to any one of the aspects and embodiments.

Suitable host cells and/or subjects for use in the methods and compositions disclosed herein include, but are not limited to, prokaryotic and eukaryotic species. Suitable host cells to be modified using the compositions and methods according to the present disclosure can include, but not limited to, algal cells, bacterial cells, heterokonts, fungal cells, chytrid cells, microfungi, microalgae, and animal cells. In some embodiments, the animal cells are invertebrate animal cells. In some embodiments, the vertebrate animal cells are mammalians cells. Host cells can be either untransformed cells or cells that have already been transfected with at least one nucleic acid molecule. Accordingly, biological samples, biomass, and progeny of a recombinant cell according to any one of the aspects and embodiments are also within the scope of the present application. Thus, as discussed in more detail below, polypeptides produced by a method according to this aspect of the application are also within the scope of this application.

In some embodiments, the recombinant cell is an animal cell. Therapeutic protein production in small and large scale is important field of development in pharmaceutical industry, because proteins produced in animal cells are believe to generally have proper processing, post-translational modification and therefore have adequate activity for treatment of the physiological condition. In principle, any animal species can be generally used and can be, for example, human, dog, bird, fish, horse, pig, primate, mouse, cotton rat, ferret, cattle, swine, sheep, rabbit, cat, goat, donkey, hamster, or buffalo. Non-limiting examples of suitable bird species include chicken, duck, goose, turkey, ostrich, emu, swan, peafowl, pheasant, partridge, and guinea fowl. In some particular embodiments, the fish is any species in the Salmonidae family. Primary mammalian cells and continuous/immortalized cells types are also suitable. Non-limiting examples of suitable animal host cells include, but not limited to, pulmonary equine artery endothelial cell, equine dermis cell, baby hamster kidney (BHK) cell, rabbit kidney cell, mouse muscle cell, mouse connective tissue cell, human cervix cell, human epidermoid larynx cell, Chinese hamster ovary cell (CHO), human HEK-293 cell, mouse 3T3 cell, Vero cell, Madin-Darby Canine Kidney Epithelial Cell (MDCK), primary chicken fibroblast cell, a HuT78 cell, A549 lung cell, HeLa cell, PER.C6® cell, WI-38 cell, MRC-5 cell, FRhL-2, and CEM T-cell. In some embodiments, the host cell is baby hamster kidney cell. In some embodiments, the baby hamster kidney cell is a BHK-21 cell.

### Recombinant Polypeptides

Some embodiments disclosed herein relate to recombinant polypeptides produced by a method in accordance with one or more embodiments described herein. The recombinant polypeptides of the present application generally can be any recombinant polypeptides and can be, for example, one or more of therapeutic polypeptides, prophylactic polypeptides, diagnostic polypeptides, nutraceutical polypeptides, industrial enzymes, and reporter polypeptides. In some embodiments, the recombinant polypeptides can be one or more of antibodies, antigens, immune modulators, and cytokines. In some embodiments, the polypeptide of interest may have therapeutic or prophylactic activity.

### Compositions and Formulations

Some embodiments disclosed herein relate to a composition comprising any of the recombinant polypeptides described herein. The composition can be, for example, a nutraceutical composition, a prophylactic composition, a pharmaceutical composition comprising a pharmaceutically acceptable carrier, or a mixture thereof. In some embodiments, the compositions of the present application can be used as a vaccine.

Some embodiments disclosed herein relate to a composition including any of the nucleic acid molecules (e.g., expression vectors) described herein. The composition can be, for example, a nutraceutical composition, a prophylactic composition, a pharmaceutical composition comprising a pharmaceutically acceptable carrier, or a mixture thereof. In some embodiments, the compositions of the present application can be used as a vaccine.

Some embodiments disclosed herein relate to a composition including any of the recombinant cells described herein. The composition can be, for example, a nutraceutical composition, a prophylactic composition, a pharmaceutical composition comprising a pharmaceutically acceptable carrier, or a mixture thereof. In some embodiments, the compositions of the present application can be used as a vaccine.

As used herein, the term “pharmaceutically-acceptable carrier” means a carrier that is useful in preparing a pharmaceutical composition or formulation that is generally safe, non-toxic, and neither biologically nor otherwise undesirable, and includes a carrier that is acceptable for veterinary use as well as human pharmaceutical use. In some embodiments, a pharmaceutically acceptable carrier is as simple as water, but it can also include, for example, a solution of physiological salt concentration. In some embodiments, a pharmaceutically acceptable carrier can be, or may include, stabilizers, diluents and buffers. Suitable stabilizers are for example SPGA, carbohydrates (such as dried milk, serum albumin or casein) or degradation products thereof. Suitable buffers are for example alkali metal phosphates. Diluents include water, aqueous buffers (such as buffered saline), alcohols and polyols (such as glycerol). For administration to animals or humans, the composition according to the present application can be given by any enteral or parenteral route, which includes inter alia intranasally, by spraying, intradermally, subcutaneously, orally, by aerosol, intramuscularly, or any combination thereof.

In some embodiments, the nucleic acid molecules (e.g., mRNAs and/or expression vectors), protein molecules, and/or compositions of the disclosure are in suitable formulations, for example pharmaceutical formulations. Provided herein include pharmaceutical formulations containing one or more of the molecules and/or compositions disclosed herein in a pharmaceutically acceptable vehicle. Some embodiments of the disclosure relate to pharmaceutical formulations comprising one or more of the expression vectors disclosed herein. Some embodiments of the disclosure relate to pharmaceutical formulations containing one or more of the nucleic acid molecules disclosed herein. Some embodiments of the disclosure relate to pharmaceutical formulations containing one or more of the polypeptides disclosed herein. Some embodiments of the disclosure relate to pharmaceutical formulations containing one or more of the recombinant cells disclosed herein.

The molecules (e.g., protein and nucleic acid molecules) and compositions disclosed herein can be in various formulations, for example pharmaceutical formulations. For example, the nucleic acid molecules (e.g., replicons, mRNAs and expression vectors), protein molecules, and/or compositions of the disclosure can be formulated, for example into a pharmaceutical formulation, with one or more covalent compounds (e.g., via direct linkage), non-covalent compounds (e.g., via charged based associations from LNPs or cationic nano-emulsions), physical compositions (e.g., vault proteins, non-charged lipid encapsulations), pharmaceutically acceptable buffers (e.g., saline, lactated Ringer's), and any combinations thereof. Many methods, reagents, and systems suitable for generating the foregoing pharmaceutical formulations are known in the art.

In some embodiments, molecules and/or compositions disclosed herein is formulated in a saline or a lipid formulation. The lipid formulation can be selected from, but is not limited to, liposomes, lipoplexes, copolymers such as PLGA, and lipid nanoparticles.

### Particles and Nanoparticles

In some embodiments, one or more of the nucleic acid molecules, polypeptide molecules, and/or compositions disclosed herein can be incorporated into particles or nanoparticles. Particles comprising one or more of the molecules and compositions disclosed herein can be polymeric particles, lipid particles, solid lipid particles, self-assembled particles, composite nanoparticles of conjugate phospholipids, surfactants, proteins, polyaminoacids, inorganic particles, or combinations thereof (e.g., lipid stabilized polymeric particles). In some embodiments, the molecules and/or compositions disclosed herein are substantially encapsulated or partially encapsulated in the particles. In some embodiments, the molecules and/or compositions disclosed herein are deposited and/or absorbed on the surface of the particles. In some embodiments, the molecules and/or compositions disclosed herein are incorporated in the particles. In some embodiments, the molecules and/or compositions disclosed herein are part of or a component of the particle. The molecules and/or compositions of the disclosure can be, in some embodiments, attached to the surface of the particles with covalent bonds, or non-covalent interactions. In some embodiments, the molecules and/or compositions of the disclosure self-assemble into a particle.

As used herein, the term “encapsulate” means to enclose, surround or encase. As it relates to the formulation of the molecules and/or compositions of the present disclosure, encapsulation may be substantial, complete or partial. The term “substantially encapsulated” means that at least greater than 50%, 60%, 70%, 80%, 85%, 90%, 95%, 96%, 97%, 98%, 99%, 99.9%, 99.99%, or 99.999% of the molecules and/or compositions of the present disclosure may be enclosed, surrounded or encased within the particle. “Partially encapsulation” means that less than 10%, 15%, 20%, 30%, 40%, 50% of the molecules and/or compositions of the present disclosure may be enclosed, surrounded or encased within the particle. For example, at least 1%, 5%, 10%, 20%, 30%, 40%, 50%, 60%, 70%, 80%, 85%, 90%, 95%, 96%, 97%, 98%, 99%, 99.9%, 99.99%, or 99.999% of the molecules and/or compositions of the present disclosure are encapsulated in the particle. Encapsulation may be determined by any known method.

In some embodiments, the particles are polymeric particles or contain a polymeric matrix. The particles can generally contain any of the polymers known in the art. The particles will generally contain one or more biocompatible polymers. The polymers can be biodegradable polymers. The polymers can be hydrophobic polymers, hydrophilic polymers, or amphiphilic polymers. In some embodiments, the particles contain one or more polymers having an additional targeting moiety attached thereto. In some embodiments, the particles are inorganic particles, such as but not limited to, gold nanoparticles and iron oxide nanoparticles.

The size of the particles can be adjusted for the intended application. The particles can be nanoparticles or microparticles. The particle can have a diameter of about 10 nm to about 10 microns, about 10 nm to about 1 micron, about 10 nm to about 500 nm, about 20 nm to about 500 nm, or about 25 nm to about 250 nm. In some embodiments the particle is a nanoparticle having a diameter from about 25 nm to about 250 nm. In some embodiments, the particle is a nanoparticle having a diameter from about 50 nm to about 150 nm. In some embodiments, the particle is a nanoparticle having a diameter from about 70 nm to about 130 nm. In some embodiments, the particle is a nanoparticle having a diameter of about 100 nm. It is understood by those in the art that a plurality of particles will have a range of sizes and the diameter is understood to be the median diameter of the particle size distribution.

In some embodiments, the molecules and/or compositions disclosed herein may be incorporated into particles that are responsive to temperature, pH, and ionic conditions. For example, the particles may comprise an ionizable network of covalently cross-linked homopolymeric ionizable monomers wherein the ionizable network is covalently attached to a single terminal region of an amphiphilic copolymer to form a plurality of “dangling chains” and wherein the “dangling chains” of amphiphilic copolymer form immobile intra-network aggregates in aqueous solution, as disclosed in U.S. Pat. No. 7,204,997.

### Liposomes, Lipoplexes, and Lipid Nanoparticles (LNPs)

The molecules and/or compositions of the disclosure can be formulated using one or more liposomes, lipoplexes, and/or lipid nanoparticles. In one embodiment, pharmaceutical formulations of the molecules and/or compositions of the disclosure include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, the molecules and/or compositions of the disclosure may be formulated in a lipid vesicle which may have crosslinks between functionalized lipid bilayers. In some embodiments, the molecules and/or compositions of the disclosure may be formulated in a lipid-polycation complex. The formation of the lipid-polycation complex may be accomplished by methods known in the art. As a non-limiting example, the polycation may include a cationic peptide or a polypeptide such as, but not limited to, polylysine, polyornithine and/or polyarginine and the cationic peptides. In some embodiments, the nucleic acid molecules and/or compositions disclosed herein may be formulated in a lipid-polycation complex which may further include a neutral lipid such as, but not limited to, cholesterol or dioleoyl phosphatidylethanolamine (DOPE). The liposome formulation may be influenced by, but not limited to, the selection of the cationic lipid component, the degree of cationic lipid saturation, the nature of the PEGylation, ratio of all components and biophysical parameters such as size.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In another embodiment, the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200, and DLin-KC2-DMA.

In some embodiments, LNP formulations described herein may comprise a polycationic composition. In some embodiments, the LNP formulations comprising a polycationic composition may be used for the delivery of the modified RNA described herein in vivo and/or ex vitro. In some embodiments, the LNP formulations described herein may additionally comprise a permeability enhancer molecule. The nanoparticle formulations may be a carbohydrate nanoparticle comprising a carbohydrate carrier and a modified nucleic acid molecule (e.g., mRNA). As a non-limiting example, the carbohydrate carrier may include, but is not limited to, an anhydride-modified phytoglycogen or glycogen-type material, phytoglycogen octenyl succinate, phytoglycogen beta-dextrin, and anhydride-modified phytoglycogen beta-dextrin.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

Additional information regarding cationic lipids suitable for LNP formulations can be found in, for example, U.S. Publication No. US2017/0151339, which is herein incorporated by reference in its entirety.

The molecules and/or compositions of the disclosure can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so that delivery of the molecules and/or compositions of the disclosure may be enhanced.

Pharmaceutical formulations of the present disclosure may additionally comprise one or more pharmaceutically acceptable excipients, which, as used herein, includes any and all solvents, dispersion media, diluents, or other liquid vehicles, dispersion or suspension aids, surface active agents, isotonic agents, thickening and emulsifying agents, preservatives, solid binders, lubricants, and the like, as suited to the particular dosage form desired. More information in this regard can be found in Remington's The Science and Practice of Pharmacy, 21st Edition, A. R. Gennaro (Lippincott, Williams & Wilkins, Baltimore, Md., 2006) which discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

### EXAMPLES

Additional alternatives are disclosed in further detail in the following examples, which are not in any way intended to limit the scope of the claims.

### Example 1

### General Experimental Procedure

### DNA Template Preparation

Plasmid DNA templates were purified (Qiagen Cat. no. 12163) from 300 mL of saturated E. coli TransforMax Epi300 (Epicentre Cat. no. EC300105) cultures grown in LB broth media (Teknova Cat. no. L8000 06) supplemented with 50 ng/ml carbamicilin (Teknova Cat. no. NC9730116). Plasmid DNA was linearized by Not-I digestion (New England Biolabs NEB cat. no. R3189S) for one hour at 37° C. Linearized template DNA was then re-purified (Zymo Cat. no. D4003), and analyzed by 0.8% agarose gel (Life Technologies Cat. no. G5018-08) against a commercial 2-log DNA ladder (New England Biolabs, NEB Cat. no. N3200S). The presence of a single band was confirmed in each sample, corresponding to the expected fragment size of the linear DNA template, prior to proceeding with ex vitro transcription.

### Ex Vitro Transcription

Ex vitro transcription (IVT) reactions were performed using 1 μg of DNA template prepared as described above, in a 20 μl reaction over a one hour incubation at 37° C. (NEB cat. no. E2065S). 1 Unit of DNase I, provided by the supplier was then added directly to the IVT reaction, and incubated at 37° C. for an additional 15 mins. Reactions were then placed on ice, and purified using the manufactures suggested method (Qiagen Cat. no. 74104). Purified RNA was then quantified using a NanoDrop 2000c UV-Vis Spectrophotometer. RNA was visualized by electrophoresis through 0.8% Agarose gels (Life Technologies Cat. no. G5018-08) and compared with Millennium RNA Marker (Ambion Cat. No. AM7150), prior to proceeding with electroporation.

### Transfection and Analysis

In a typical cell transfection experiment, replicon RNA was introduced into BHK-21 cells by electroporation using the SF Cell Line Nucleofector™ kit for the 4D-Nucleofector™ System (Lonza). BHK-21 cells were harvested using 0.25% trypsin and washed once with cold PBS. Cells were resuspended in SF Buffer at a cell density of 1×10⁶ cells per 20 μL electroporation reaction. Three micrograms of RNA was electroporated into cells in triplicate in a 16-well cuvette strip and incubated at room temperature for 10 minutes. Electroporated cells were recovered into plates containing Dulbecco's Modified Eagle Medium containing 10% fetal bovine serum, followed by incubation for 16-18 h at standard cell culture conditions.

Intracellular analyses of replicon transfection efficiency and protein production were performed by flow cytometry. In these assays, transfected BHK-21 cells were fixed and permeabilized using fix/perm concentrate and permeabilization buffer (eBioscience). Cells were then incubated with antibodies for double-stranded RNA production (J2 anti-dsRNA IgG2A monoclonal antibody, English & Scientific Company) conjugated with R-Phycoerythrin (Innova Biosciences). Antigen production was assessed by additional incubation with antigen-specific antibodies conjugated with PE-Cy5 (Innova Biosciences) (e.g. antibodies for red Firefly, green Renilla, HA, or RSV-F0 (Abcam)). Cells were then washed once and analyzed using a FACSAria™ Fusion Cell Sorter (BD Biosciences) or FACSAria™ II Cell Sorter (BD Biosciences). Transfected BHK-21 cells stained with single colors for compensation controls were run prior to sample collection. Data was collected using FACSDiva (BD Biosciences) and further analyzed using FlowJo software. Initial gating was performed to exclude dead cells and debris using forward and side scatter plots. Further gating was conducted to identify cell populations that were positive for both dsRNA (R-PE-positive) and protein expression (PE-Cy5-positive or FITC-positive for GFP expression). Frequencies and mean fluorescence intensities were collected and utilized for construct comparison and optimization.

### Example 2

### Construction of DLP-Containing EAV Replicon Designs

This Example describes the generation of a number of arterivirus RNA replicon-based expression vectors with a DLP motif operably positioned upstream of the polyprotein/non-structural protein genes and/or a reporter gene. These arterivirus RNA replicon-based expression vectors were subsequently characterized and analyzed in the flow cytometry analysis and bulk luciferase analyses described in EXAMPLE 4.

### A. Design

The respective design features of four EAV-based DLP replicon constructs are described below.

(1) rEX-DLP-rFF

In this construct, a DLP motif as placed immediately upstream of rFF and downstream of the TRS7 driving the transcription of rFF.

(2) rEX-DLP-pp1 ab-rFF

In this construct, a DLP motif was placed immediately upstream of the pp1ab genes with a few careful design modifications described below to maintain the stem loop structure in the 5′UTR of the replicon known to be essential for replication and subgenomic mRNA transcription.

(i) The first 79 nucleotides of the nonstructural viral gene 1a is duplicated with its start codon mutated from ATG to TAG, denoted as “ATG-shifting region” (bold in the sequence of SEQ ID NO: 2 below).

(ii) The corresponding nucleotides, located upstream of the 1a gene, base-pairing with its start codon ATG and forming the stem, were also changed accordingly from CAT to CTA (underlined in the sequence of SEQ ID NO: 2 below).

(iii) DLP (italicized in the sequence below) was placed immediately downstream of the “ATG-shifting region” and upstream of the polyprotein 1ab genes (start codon ATG shown in the sequence of SEQ ID NO: 2 below).

(partial sequence)
SEQ ID NO: 2
CGAAGTGTGTATGGTGCCATATACGGCTCACCACCATATACACTGCAA
GAATTACTATTCTTGTGGGCCCCTCTCGGTAAATCCTAGAGGGCTTTC
CTCTCGTTATTGCGAGATTCGTCGTTAGATAACGGCAAGTTCCCTTTC
TTACTATCCTATTTTCATCTTGTGGCTTGACGGGTCACTGCCTACGTC
GTCGATCTCTATCAACTACCCTTGCGACTTAGGCAACCTTCTCCGCTA
CTGGATTTGGAGGGAGTTTTGTTAGGGACTGGTCCCTGGACTTACCCG
ACGCTTGTGAGCATAGTCAGCATAGTACATTTCATCTGACTAATACTA
CAACACCACCACCATGAATAGAGGATTCTTTAACATGCTCGGCCGCCG
CCCCTTCCCGGCCCCCACTGCCATGTGGAGGCCGCGGAGAAGGAGGCA
GGCGGCCCCGATGATGGCAACCTTCTCCGCTACTGGATTTGGAGG . . .

This construct was essentially identical to the second construct, where DLP was placed following the same three design modifications, except that a 2A protease sequence (SEQ ID NO: 3) was added immediately at the 3′ end of DLP such that, when translated, the polyproteins could be released from the DLP-derived peptide through a selective cleavage by the protease. A comparative analysis of performances by replicon Construct 2 (described above) and Construct 3 would provide information on whether the 2A protease was needed for a functional replicon (see EXAMPLE 4 below).

SEQ ID NO: 3
GGAAGCGGAGCTACTAACTTCAGCCTGCTGAAGCAGGCTGGAGACGTGG
AGGAGAACCCTGGACCT

(4) rEX-DLP-2A-pp1ab-DLP-rFF

This construct was essentially identical to the third construct described above, except that another DLP was placed immediately upstream of the reporter rFF gene (the same way as a DLP motif was placed in construct 1). A comparative analysis of performances by replicon Construct 3 (described above) and Construct 4 would provide information on whether the additional DLP placed upstream of the reporter gene has an added value to the expression of the reporter gene.

### B. Construction

rEx-DLP-rFF was built according to a 3-piece Gibson Assembly® procedure described in Gibson et al. (Enzymatic assembly of DNA molecules up to several hundred kilobases. Nat. Methods 6, 343-345, 2009) with rEx-rFF (c4; SEQ ID NO: 34) digested with SphI and EcoRI as a vector and a DLP-containing g-block as an insert. The nucleic acid sequence of the g-block used for construction of rEx-DLP-rFF is set forth at SEQ ID NO: 4 in the Sequence Listing.

The following primers were designed to amplify the corresponding fragments required to build the 3 new EAV-based DLP replicon constructs described above.

TABLE 3

| | Primers designed for construction | |
|---|---|---|
| Primer | DLP-(2A)-pp1ab-rFF/DLP-rFF replicons | |
| RP114 | pp1a-DLP-F | GCCATGTGGAGGCCGCGGAGA |
| | | AGGAGGCAGGCGGCCCCGATG |
| | | ATGGCAACCTTCTCCGCTACT |
| | | GGAT (SEQ ID NO: 5) |
| RP115 | pBR322-3′SrfI-R | ACAATGTTGCCTCCCACATCT |
| | | GCAA (SEQ ID NO: 6) |
| RP116 | pBR322-3′SrfI-F | GGGTCACAAGGTAGTCGCCGT |
| | | GGTT (SEQ ID NO: 7) |
| RP117 | pBR322-bla-R | ACGTCAGGTGGCACTTTTCGG |
| | | GGAA (SEQ ID NO: 8) |
| RP118 | pp1a-DLP-2A-F | AGCCTGCTGAAGCAGGCTGGA |
| | | GACGTGGAGGAGAACCCTGGA |
| | | CCTATGGCAACCTTCTCCGCT |
| | | ACTGGAT (SEQ ID NO: 9) |

Construction of rEx-DLP-pp1ab-rFF

For the construction of the rEx-DLP-pp1ab-rFF vector, three nucleic acid fragments were generated by using a 3-piece Gibson Assembly® procedure, as follows.

Fragment 1 was generated with primers RP114 and RP115 and the template backbone rEx-rFF.

Fragment 2 was generated with primers RP116 and RP117 and template backbone rEx-rFF.

Fragment 3 was a g-block for rEx-DLP-pp1ab-rFF with the nucleic acid sequence set forth at SEQ ID NO: 10 in the Sequence Listing.

Construction of rEx-DLP-2A-pp1ab-rFF

For the construction of rEx-DLP-2A-pp1ab-rFF vector, three nucleic acid fragments were generated by using a 3-piece Gibson Assembly® procedure, as follows.

Fragment 4 was generated with primers RP118 and RP115 and the template backbone rEx-rFF.

Fragment 5 was generated with primers RP116 and RP117 and template backbone rEx-rFF.

Fragment 6 was a g-block for rEx-DLP-2A-pp1ab-rFF with the nucleic acid sequence set forth at SEQ ID NO: 11 in the Sequence Listing.

Construction of rEx-DLP-2A-pp1ab-DLP-rFF

For the construction of rEx-DLP-2A-pp1ab-DLP-rFF vector, three nucleic acid fragments were generated by using a 3-piece Gibson Assembly® procedure, as follows.

Fragment 7 was generated with primers RP118 and RP115 and the template backbone rEx-DLP-rFF.

Fragment 8 was generated with primers RP116 and RP117 and template backbone rEx-DLP-rFF.

Fragment 9 was a g-block for rEx-DLP-2A-pp1ab-DLP-rFF with the nucleic acid sequence set forth at SEQ ID NO: 12 in the Sequence Listing.

Construct assembly was performed according to a 3-piece Gibson Assembly® procedure described in Gibson et al. (2009, supra). In particular, the rEx-DLP-pp1ab-rFF construct was built using fragments 1, 2, and 3; the rEx-DLP-2A-pp1ab-rFF construct was built using fragments 4, 5, and 6; and the rEx-DLP-2A-pp1ab-DLP-rFF construct was built using fragments 7, 8, and 9. Assembled products were subsequently transformed into EPI300 cells from Epicenter. A total of 144 colonies were screened using the primers RP126 (SEQ ID NO: 13) and RP127 (SEQ ID NO: 14) for each transformation, resulting in 4 PCR-positive clones for rEx-DLP-pp1ab-rFF, 3 PCR-positive clones for rEx-DLP-2A-pp1ab-rFF, and 2 PCR-positive clones for rEx-DLP-2A-pp1ab-DLP-rFF. Subsequent MiSeq results revealed that clone 4, clones 3 and 15, and clones 18 and 20 were completely sequence-correct for rEx-DLP-pp1ab-rFF, rEx-DLP-2A-pp1ab-rFF, and rEx-DLP-2A-pp1ab-DLP-rFF, respectively.

TABLE 4

| | Primers designed for colony screening | |
|---|---|---|
| Primer | of the DLP-(2A)-pp1ab replicons | |
| RP126 | DLP-pp1ab- | CAGCATCTTTTACTT |
| | screen-F | TCACCAGCGTTTCTG |
| | | (SEQ ID NO: 13) |
| RP127 | DLP-pp1ab- | GGAACTGGCGAAGCC |
| | screen-R | AGTTTTAACA |
| | | (SEQ ID NO: 14) |

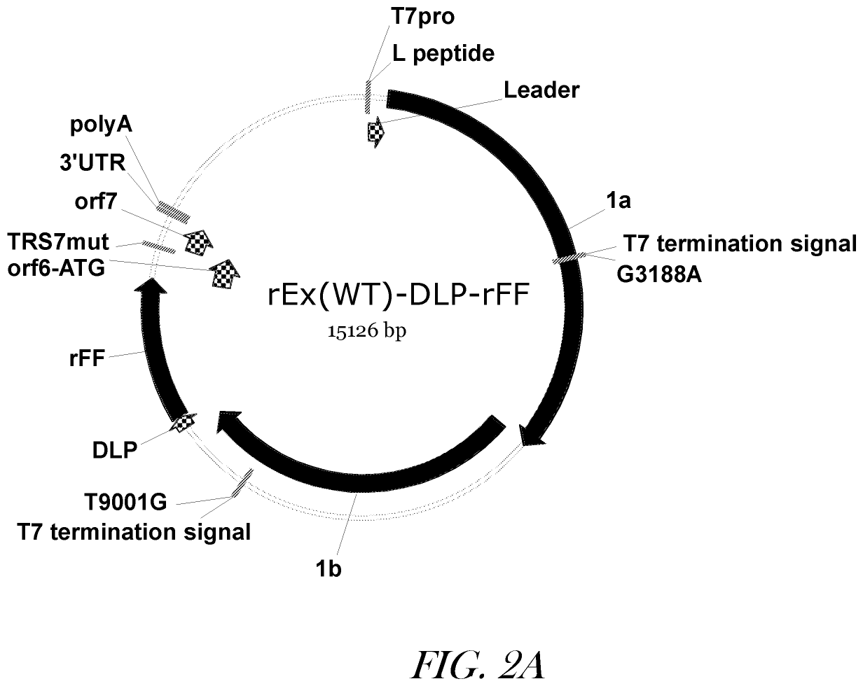

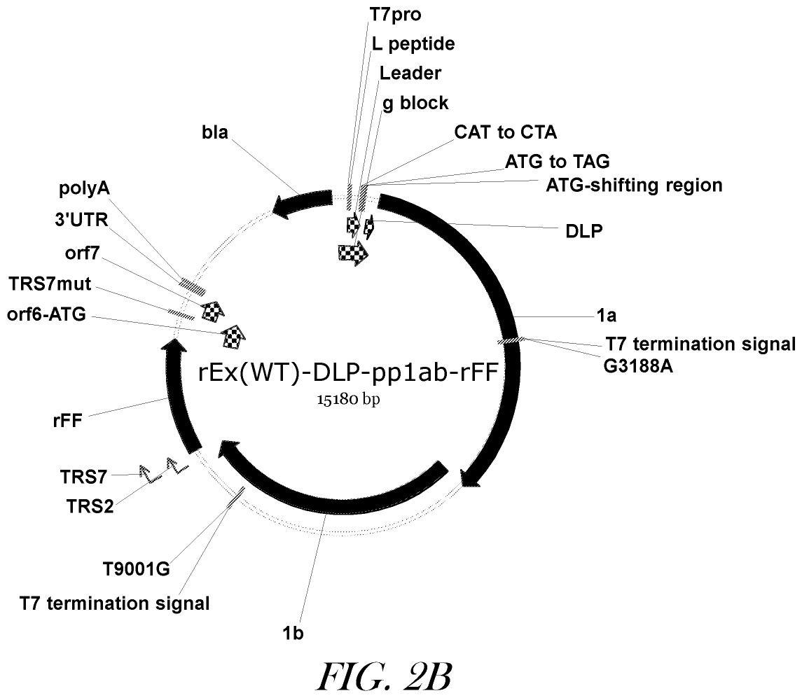

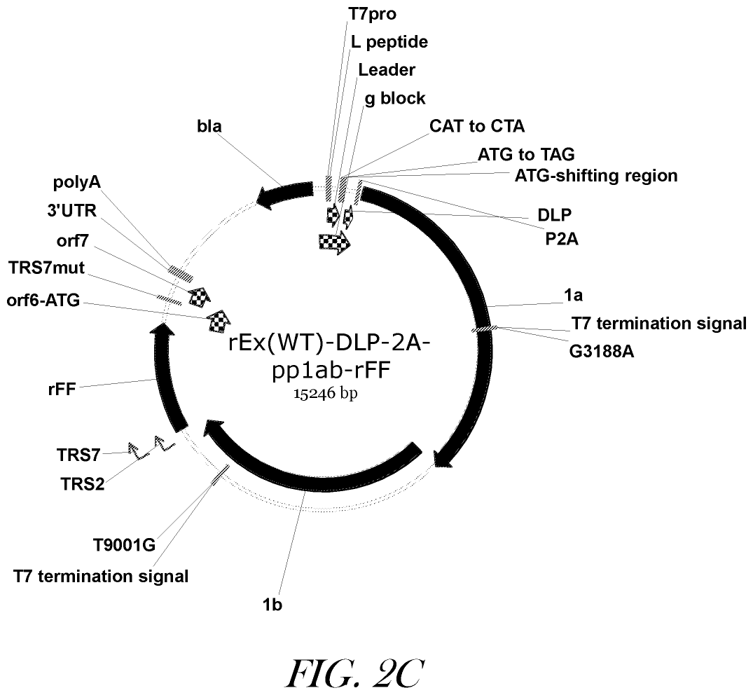

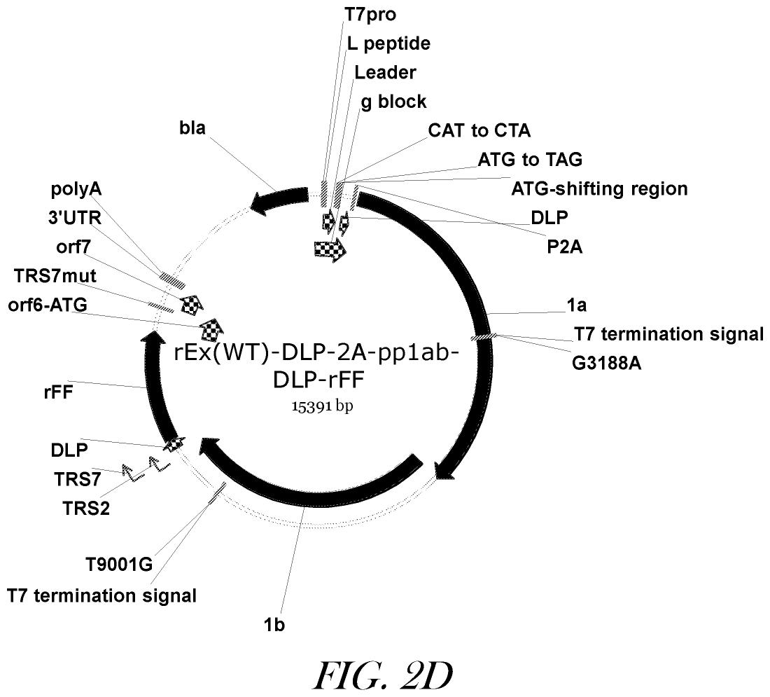

The maps of rEx-DLP-rFF, rEx-DLP-pp1ab-rFF, rEx-DLP-2A-pp1ab-rFF, and rEx-DLP-2A-pp1ab-DLP-rFF are also shown in FIGS. 2A-2D.

The sequences of the resulting replicons are disclosed in the Sequence Listing with a T7 promoter and a polyA tail of 65 A's, as follows: rEx-DLP-rFF (SEQ ID NO: 15), rEx-DLP-pp1ab-rFF (SEQ ID NO: 16), rEx-DLP-2A-pp1ab-rFF (SEQ ID NO: 17), and rEx-DLP-2A-pp1ab-DLP-rFF (SEQ ID NO: 18).

### Example 3

### Construction of DLP-Containing Alphavirus Replicon Designs

This Example describes the generation of a number of Alphavirus RNA replicon-based expression vectors with a DLP motif positioned upstream of the polyprotein/non-structural protein genes and/or a reporter gene. These Alphavirus RNA replicon-based expression vectors were subsequently characterized and analyzed in the flow cytometry analysis and bulk luciferase analyses described in EXAMPLE 5.

### A. Design

The respective design features of three Alphavirus-based DLP replicon constructs are described below.

### (1) Alpha-R-DLP-rFF

In this construct, In this construct, DLP was placed immediately upstream of the start codon of the reporter gene rFF.

### (2) Alpha-R-DLP-2A-nsp-rFF

In this construct, the sequence encoding the DLP motif and the 2A peptide sequence (which was the same sequence used in the rEx-DLP-2A-pp1ab-rFF replicon described in Example 2 above) was placed within the 5′ end of the replicon with a few careful design modifications described below, to potentially maintain the sequence-structure requirement for replication and subgenomic mRNA transcription.

(i) The first 195 nucleotides of the nsp1 gene was duplicated with its start codon mutated from ATG to TAG (bold in the sequence of SEQ ID NO: 19 below).

(ii) This 195-nucleotide duplicated sequence was placed immediately following the 5′ UTR of the wild-type Alphavirus (underlined in the sequence of SEQ ID NO: 19 below) and is followed by the DLP-2A sequence (italicized in the sequence below).

(iii) The start codon of the nsp1 gene following the DLP-2A sequence was removed (strike-through in the sequence of SEQ ID NO: 19 below).

(partial sequence)
SEQ ID NO: 19
GATAGGCGGCGCATGAGAGAAGCCCAGACCAATTACCTACCCAAATAG
GAGAAAGTTCACGTTGACATCGAGGAAGACAGCCCATTCCTCAGAGCT
TTGCAGCGGAGCTTCCCGCAGTTTGAGGTAGAAGCCAAGCAGGTCACT
GATAATGACCATGCTAATGCCAGAGCGTTTTCGCATCTGGCTTCAAAA
CTGATCGAAACGGAGGTGGACCCATCCGACACGATCCTTGACATTGGA
ATAGTCAGCATAGTACATTTCATCTGACTAATACTACAACACCACCAC
CATGAATAGAGGATTCTTTAACATGCTCGGCCGCCGCCCCTTCCCGGC
CCCCACTGCCATGTGGAGGCCGCGGAGAAGGAGGCAGGCGGCCCCGGG
AAGCGGAGCTACTAACTTCAGCCTGCTGAAGCAGGCTGGAGACGTGGA
GGAGAACCCTGGACCT [symbol] GAGAAAGTTCACG . . .

### (3) Alpha-R-DLP-2A-nsp-DLP-rFF

This construct is essentially identical to Construct 2 following the same three design modifications, except that another DLP motif was placed immediately upstream of the reporter rFF gene (the same way as a DLP motif was placed in Construct 1). A comparative analysis of performances by replicon Constructs 2 and 3 would provide information on whether the additional DLP placed upstream of the reporter gene has an added value to the expression of the reporter gene (see EXAMPLE 5 below).

### B. Construction

### Construction of Alpha-R-DLP-rFF

Alpha-R-DLP-rFF was built via Gibson Assembly® procedure, using Alpha-R-eGFP (c6; SEQ ID NO: 35) digested with EcoRI/SapI as a vector and DLP-rFF as an insert PCR-amplified from the template rEx-DLP-rFF (c2, SEQ ID NO: 15) using the primers RP112 (SEQ ID NO: 20) and RP113 (SEQ ID NO: 21) to replace eGFP with DLP-rFF. Clones 2 and 3 were sequence-confirmed to be completely correct via MiSeq sequencing.

TABLE 5

| | Primers used to clone DLP-rFF | |
|---|---|---|
| Primer | into Alpha-R-GFP (EcoRV/SapI) | |
| RP112 | DLP-rFF-F | CCTGAATGGACTACGACATAGTCTAG |
| | | TCCGCCAAGATATCGCACCATAGTCA |
| | | GCATAGTACATTTCATCTGACTAATA |
| | | CT (SEQ ID NO: 20) |
| RP113 | DLP-rFF-R | GCAGCTTGCCAATTGCTGCTGTATCG |
| | | ATCAATTAATCACATCTTGGCCACGG |
| | | GTTTCTTC (SEQ ID NO: 21) |

### Construction of Alpha-R-DLP-2A-nsp-rFF and Alpha-R-DLP-2A-nsp-DLP-rFF

Alpha-R-DLP-2A-nsp-rFF (Construct 2) and Alpha-R-DLP-2A-nsp-DLP-rFF (Construct 3) were built via Gibson Assembly© procedure, using the respective g-blocks as inserts and the vectors that had been PCR-amplified from the respective templates, Alpha-R-rFF (c6; SEQ ID NO: 35) and Alpha-R-DLP-rFF (c2; SEQ ID NO: 26), using the primers RP124 (SEQ ID NO: 22) and RP125 (SEQ ID NO: 23). Clones 1 and 3 of Alpha-R-DLP-2A-nsp-rFF and clones 8 and 32 of Alpha-R-DLP-2A-nsp-DLP-rFF were sequence-confirmed to be completely correct via MiSeq.

TABLE 6

| | Primers used for construction | |
|---|---|---|
| Primer | of Alpha-DLP-nsp-rFF/DLP-rFF | |
| RP124 | 5′Alpha- | GAAGCAGGCTGGAGACGTGGAGGAGAACCC |
| | P2A-F | TGGACCTGAGAAAGTTCACGTTGACATCGA |
| | | GGAAGAC (SEQ ID NO: 22) |
| RP125 | 5′ScaI-R | CACCAGTCACAGAAAAGCATCTTACGGATG |
| | | (SEQ ID NO: 23) |

The sequence of g-block used for the construction of Alpha-R-DLP-2A-nsp-rFF is provided in the Sequence Listing as SEQ ID NO: 24. The sequence of g-block used for the construction of Alpha-R-DLP-2A-nsp-DLP-rFF is also provided in the Sequence Listing as SEQ ID NO: 25.

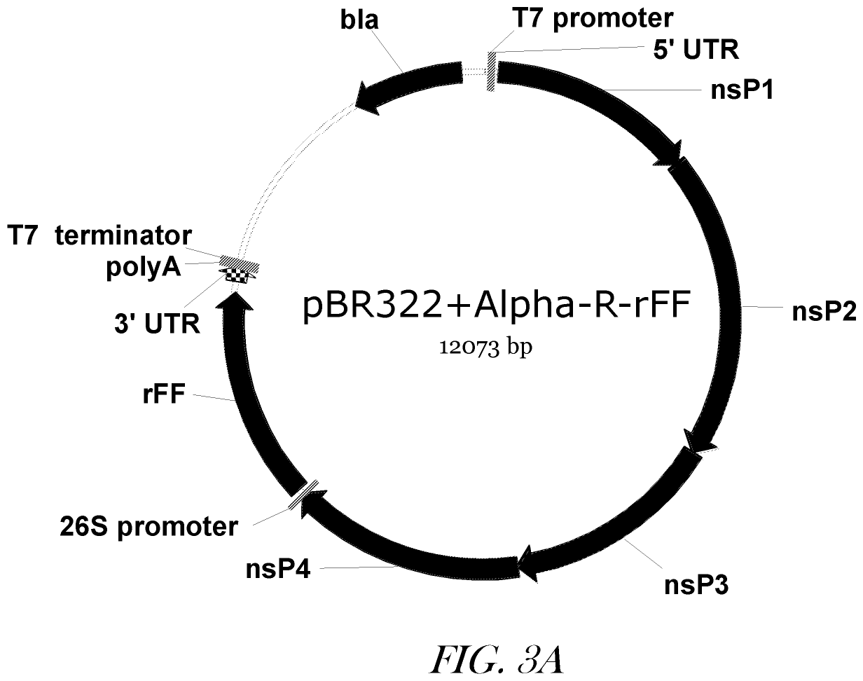

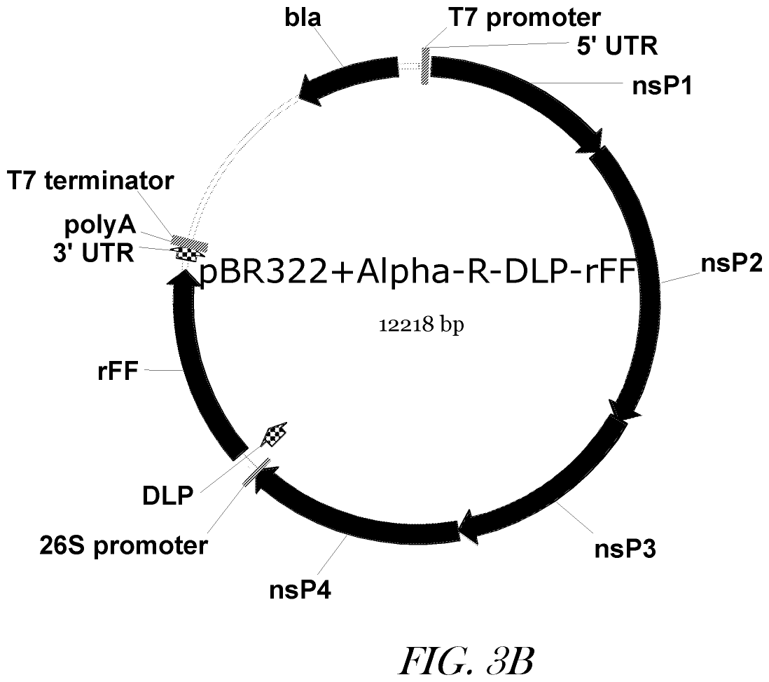

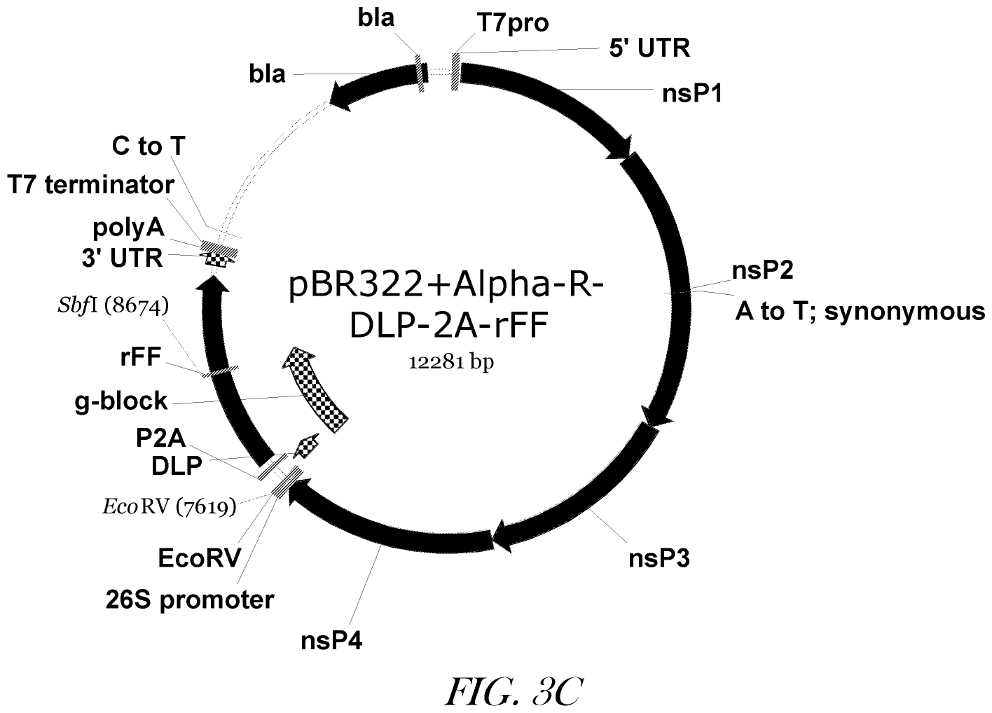

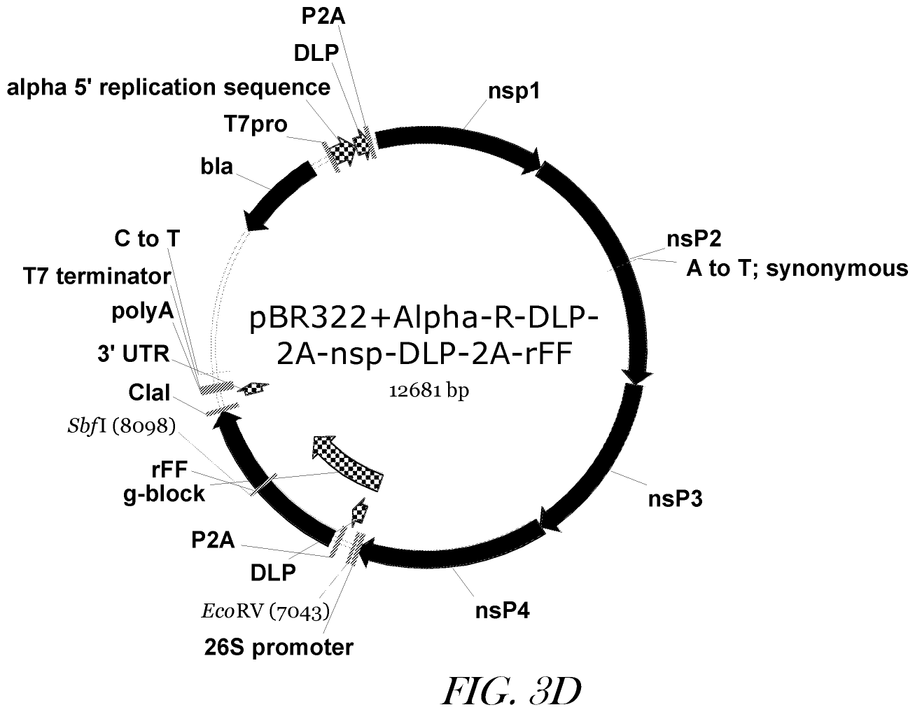

The maps of Alpha-R-rFF, Alpha-R-DLP-rFF, Alpha-R-DLP-2A-nsp-rFF, and Alpha-R-DLP-2A-nsp-DLP-rFF are shown in FIGS. 3A-3D.

The sequences of the resulting replicons are also provided in the Sequence Listing with a T7 promoter and a polyA tail of 40 A's, as follows: Alpha-R-rFF (SEQ ID NO: 26), Alpha-R-DLP-rFF (SEQ ID NO: 27), Alpha-R-DLP-2A-nsp-rFF (SEQ ID NO: 28), and Alpha-R-DLP-2A-nsp-DLP-rFF (SEQ ID NO: 29).

### Construction of Alpha-R-DLP-2A-rFF and Alpha-R-DLP-2A-nsp-DLP-2A-rFF

Without being bound by any particular theory, it is believed that placing a DLP motif immediately upstream of the reporter gene rFF without the inclusion of the 2A protease in between them may negatively impact protein expression of the GOI; this negative impact could be due to the fact that rFF now became a “fusion” protein, resulting from the presence of the DLP sequence translated into a peptide at the 5′ end of rFF. Therefore, 2 new constructs were designed and built, including the 2A protease sequence between the DLP motif and the rFF gene for the two Alphavirus-replicon constructs, Alpha-R-DLP-rFF and Alpha-R-DLP-2A-nsp-DLP-rFF, to generate Alpha-R-DLP-2A-rFF and Alpha-R-DLP-2A-nsp-DLP-2A-rFF, respectively. The inclusion of the 2A protease peptide sequence would enable cleavage of the peptide encoded by the DLP sequence from rFF (see Example 5 below).

For this purpose, two g-block fragments were synthesized (SEQ ID NOS: 30 and 31) and cloned into their respective vectors digested with EcoRV/SbfI via Gibson Assembly. Clone 1 of Alpha-R-DLP-2A-rFF and clones 8 and 9 of Alpha-R-DLP-2A-nsp-DLP-2A-rFF were sequence-confirmed to be completely correct via Sanger sequencing using RP123 (SEQ ID NO: 32) and RP96 (P89; SEQ ID NO: 96).

TABLE 7

| | Primers used to sequence Alpha-R- | |
|---|---|---|
| Primer | (DLP-2A-nsp)-DLP-2A-rFF constructs | |
| RP123 | Alpha-3′nsp4-F | GGCTGTTTAAGCTTGGCAAACCTCT |
| | | (SEQ ID NO: 32) |
| RP96 | rFF-seq1 | AGCGAGAACTGCGAGGAATTCTT |
| | | (SEQ ID NO: 33) |

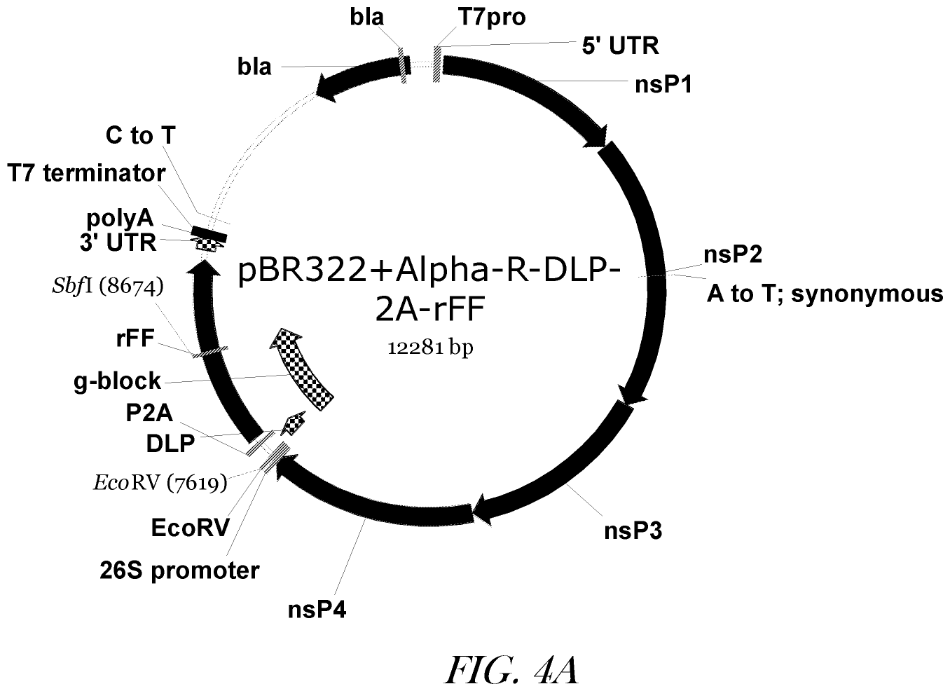

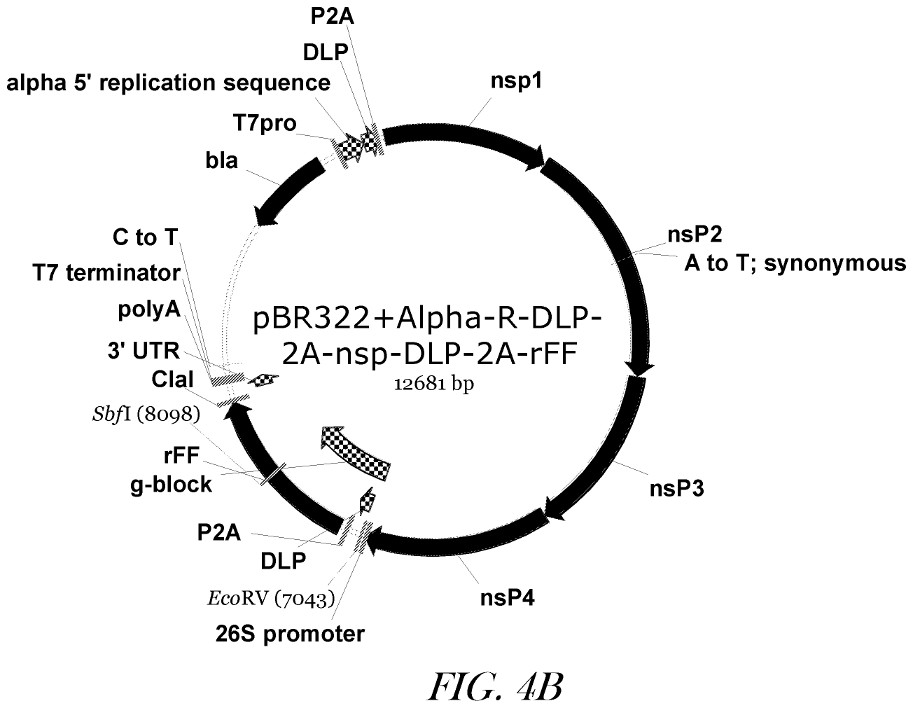

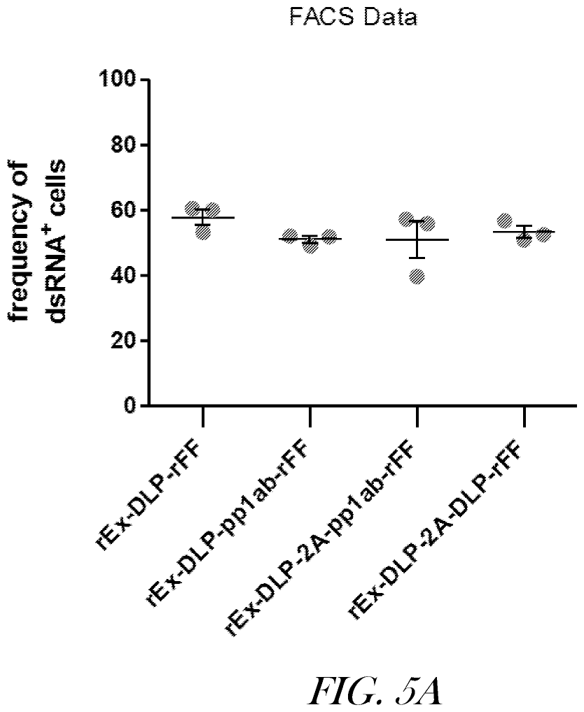

Schematic maps of Alpha-R-DLP-2A-rFF and Alpha-R-DLP-2A-nsp-DLP-2A-rFF are provided in FIGS. 4A-4B.

### Example 4

### Expression Analysis of EAV-Based DLP Containing Replicons

As presented in Examples 2 and 3 above, a number of EAV-based DLP containing replicons were constructed to determine the impact of engineering a DLP motif positioned upstream of either the replicon nonstructural protein genes or the GOI gene on a subgenomic mRNA (TABLE 8).

TABLE 8
Listing of DLP-containing EAV Replicons
and DLP-containing VEEV replicons.

EAV DLP Replicons
rEx-DLP-rFF
rEx-DLP-2A-rFF
rEx-DLP-pp1ab-rFF
rEx-DLP-2A-pp1ab-rFF
rEx-DLP-2A-pp1ab-DLP-rFF
rEx-DLP-2A-pp1ab-DLP-2A-rFF
VEEV DLP replicons
alpha-R-DLP-rFF
alpha-R-DLP-2A-rFF
alpha-R-DLP-2A-nsp-rFF
alpha-R-DLP-2A-nsp-DLP-rFF
alpha-R-DLP-2A-nsp-DLP-2A-rFF

Initial characterization of the DLP replicon constructs was carried out ex vitro. RNA was produced and used to electroporate BHK cells as described in EXAMPLE 1 above. After electroporation cells were analyzed for protein expression by FACs analysis, Western blot or bulk luciferase assay.

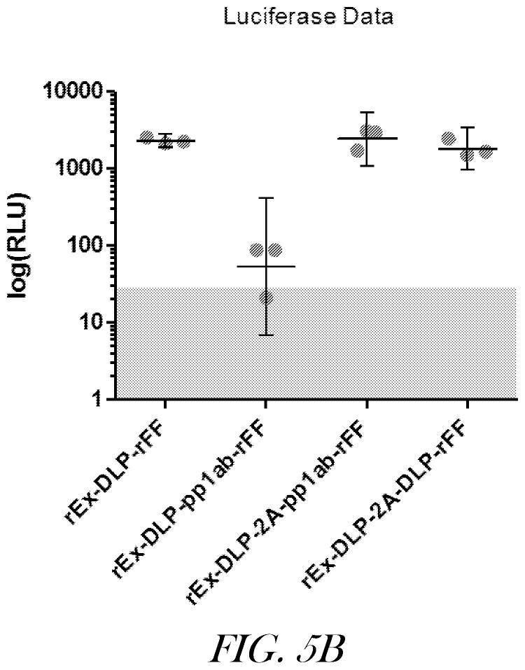

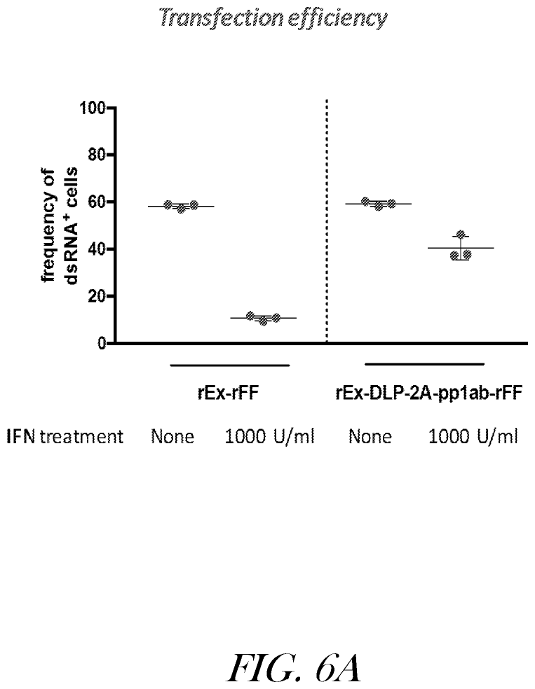

A graphical summary of the results of experiments performed to measure the expression level of an exemplary gene of interest (GOI), rFF luciferase reporter, from EAV-based DLP replicons is shown in FIG. 5. Both FACs analysis and bulk luciferase data are presented. In these experiments, four different EAV DLP replicons were analyzed as follows:

1) rEx-DLP-rFF: an EAV-based replicon with a DLP motif positioned upstream to the subgenomic mRNA rFF transcript);

2) rEx-DLP-pp1ab-rFF: an EAV-based replicon with DLP positioned upstream to the non-structural pp1ab genes);

3) rEx-DLP-2A-pp1ab-rFF: an EAV-based replicon with a DLP motif positioned upstream to the nonstructural proteins and a 2A protease peptide positioned between the DLP and the pp1ab region); and

4) rEx-DLP-2A-pp1ab-DLP-rFF: an EAV-based replicon with a first DLP motif positioned upstream to the nonstructural proteins and a 2A protease peptide positioned between the DLP and the pp1ab region as well as a second DLP motif positioned upstream to the rFF subgenomic mRNA transcript).

The results presented in FIGS. 5A-5B demonstrated that engineering a DLP motif upstream to either the EAV nonstructural protein genes (e.g., rEx-DLP-pp1ab-rFF, rEx-DLP-2A-pp1ab-rFF or rEx-DLP-2A-pp1ab-DLP-rFF) or the rFF reporter gene subgenomic RNA (e.g., rEx-DLP-rFF and rEx-DLP-2A-pp1ab-DLP-rFF) did not negatively impact genomic RNA replication as all four constructs demonstrated nearly identical electroporation efficiencies (FIG. 5A). Interestingly, bulk luciferase activity analysis demonstrated that the rEx-DLP-pp1ab-rFF replicon expressed significantly less luciferase than the other three replicon designs (FIG. 5B). As stated above, incorporation of a DLP motif upstream of any GOI would result in an N terminal fusion of Sindbis capsid amino acids encoded in the in-frame codons found in the DLP sequence. The fusion protein generated with the amino acids encoding DLP and the EAV nsP1 protein is believed to impact the EAV replication complex from efficiently producing subgenomic RNAs and result in the reduced rFF GOI expression levels noted. One of the most remarkable results from this study was that EAV replicon constructs with a DLP controlling translation of the nonstructural protein genes (rEx-DLP-pp1ab-rFF, rEx-DLP-2A-pp1ab-rFF and rEx-DLP-2A-pp1ab-DLP-rFF) were as efficiently translated as the replicon RNA that did not have a DLP in this position (rEx-DLP-rFF). This result would not be predicted based on work conducted by other researchers. It has been previously reported that incorporation 5′ Sindbis virus subgenomic RNA sequences (including the DLP region) were only efficiently translated in cells infected with the virus. Stated differently, mRNA that contains a DLP motif associated with a reporter gene was reported to be poorly translated in cells that were not infected with Sindbis virus. The absence of innate immune activation in these cells rendered the DLP modified mRNA at a distinct translation disadvantage relative to translation of mRNAs that lack the DLP modification (all cellular mRNAs). The innate immune system was not activated in these cells at the time the DLP-containing replicon vectors were introduced so these DLP-containing mRNAs (capable of self-amplification) should be very inefficiently translated. Unexpectedly, that was not borne out in the experiments presented herein.

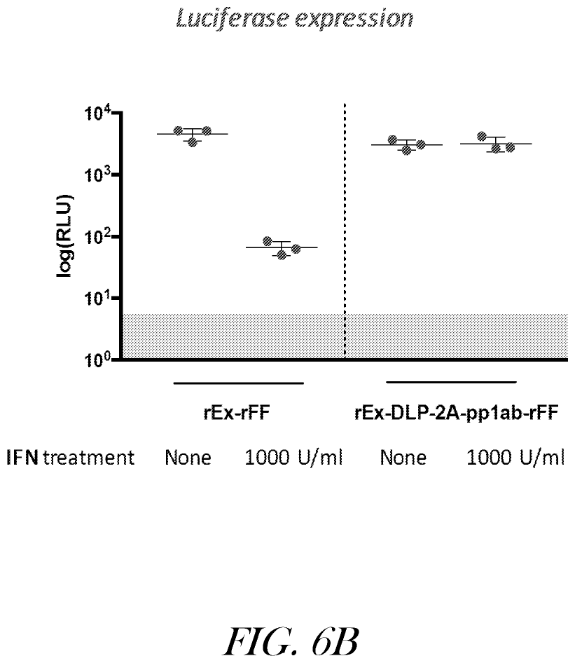

Subsequently, the rEx-DLP-2A-pp1ab-rFF EAV replicon was examined in cells that had been treated with IFN to induce the cellular innate immune system. IFN treatment of BHK cells will induce PKR activation and phosphorylation of eIF2a which in-turn results in shut-down of global cellular mRNA translation. It has been reported previously that arteriviruses are sensitive to IFN treatment (Luo et al. Antiviral Res. August; 91(2):99-101, 2011), therefore the IFN treatment of BHK cells, which are capable of responding to IFN exposure and induce the innate immune system, would result in shut-down of arterivirus replication. A representative example of the expression capacity of the DLP modified EAV replicon in the presence of innate immune system activation is shown in FIG. 6. The rEx-DLP-2A-pp1ab-rFF replicon demonstrated significant resistance to innate immune system activation when compared to an EAV replicon that was not modified to contain the DLP motif, i.e. rEx-rFF. Both replication (FIG. 6A) and expression (FIG. 6B) of the rEx-DLP-2A-pp1ab-rFF replicon were significantly higher in IFN treated cells when compared to the control rEx-rFF replicon. These data demonstrate that DLP modified EAV replicons are capable of overcoming innate immune system shut-down and that this replicon vector represents a significant advance in self-amplifying RNA technology.

### Example 5

### Expression Analysis of DLP-Containing VEEV Replicons

As presented in Examples 2 and 3 above, a number of VEEV-based DLP containing replicons were constructed to determine the impact of engineering a DLP motif positioned upstream of either the replicon nonstructural protein genes or the GOI gene on a subgenomic mRNA.

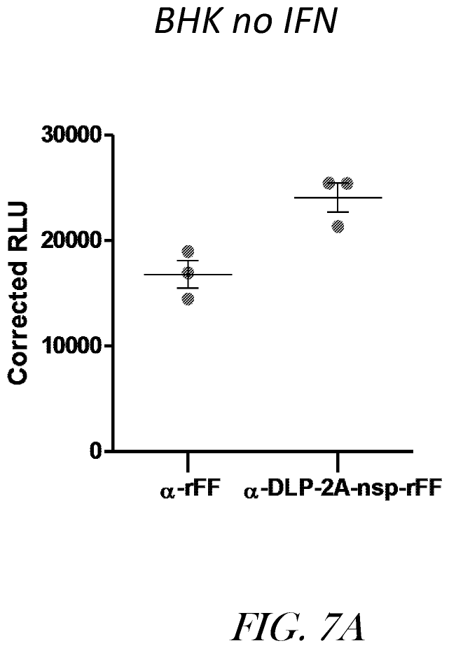

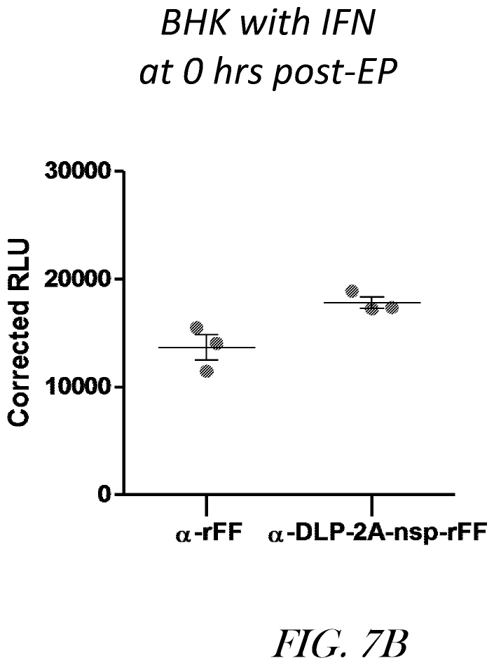

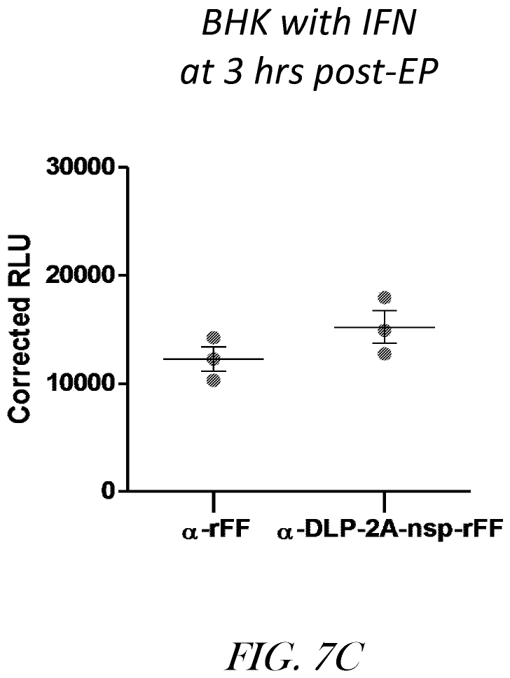

VEEV alphavirus replicon vectors were engineered to contain one or more DLP motifs by using a strategy similar to the construction of EAV-based replicon vectors. Importantly, unlike other members of the Alphavirus genus (mostly Old World virus members), the genome of VEEV does not contain a DLP motif associated with translation of its subgenomic mRNA. Initial analysis of the VEEV DLP replicons was carried out in BHK-21 cells as described in EXAMPLE 1 above. BHK-21 cells do not secrete IFN in response to RNA replication but these cells are able to respond to exogenous IFN to induce innate immune activation. In this experiment, four different alphavirus replicon constructs were tested. The experimental data presented in FIG. 7 shows DLP-containing alphavirus replicon replication and expression of the rFF luciferase gene in BHK cells that had been treated either at the time of electroporation (0 hr) or at 3 hr post electroporation with 1000 U/ml of exogenous IFN. The replicon RNAs tested were:
1) Alpha-R-rFF: a control VEEV-based replicon with no DLP present;
2) Alpha-R-DLP-rFF: a VEEV-based replicon with a DLP motif positioned upstream to the subgenomic mRNA rFF transcript;
3) Alpha-R-DLP-2A-nsp-rFF: a VEEV-based replicon with a DLP motif positioned upstream to the nonstructural proteins with a 2A protease between the DLP and the nsp region; and
4) Alpha-R-DLP-2A-nsp-DLP-rFF: VEEV-based replicon with a first DLP motif positioned upstream to the nonstructural proteins with a 2A protease between the DLP and the nsp region as well as with a second DLP motif positioned upstream to the rFF subgenomic mRNA transcript.

The results of luciferase expression normalized to the number of positive cells detected by FACs analysis are shown in FIG. 7. It was observed that the presence of a DLP motif controlling the translation of the VEEV non-structural protein genes resulted in higher reporter gene expression both in the absence and the presence of IFN treatment post electroporation (FIG. 7A-7C). Although the increase in rFF expression may have been considered statistically insignificant, the trend in all conditions was for increased protein expression. As stated above in EXAMPLE 4 with respect to DLP-containing EAV replicons, one may have expected that a DLP motif would have a negative impact on mRNA translation in cells that are not in an innate immune response activated state. In direct contrast to that expectation, the BHK cells that had not been treated with IFN (FIG. 7A) in these experiments represent the sample with the largest benefit to incorporation of a DLP motif.

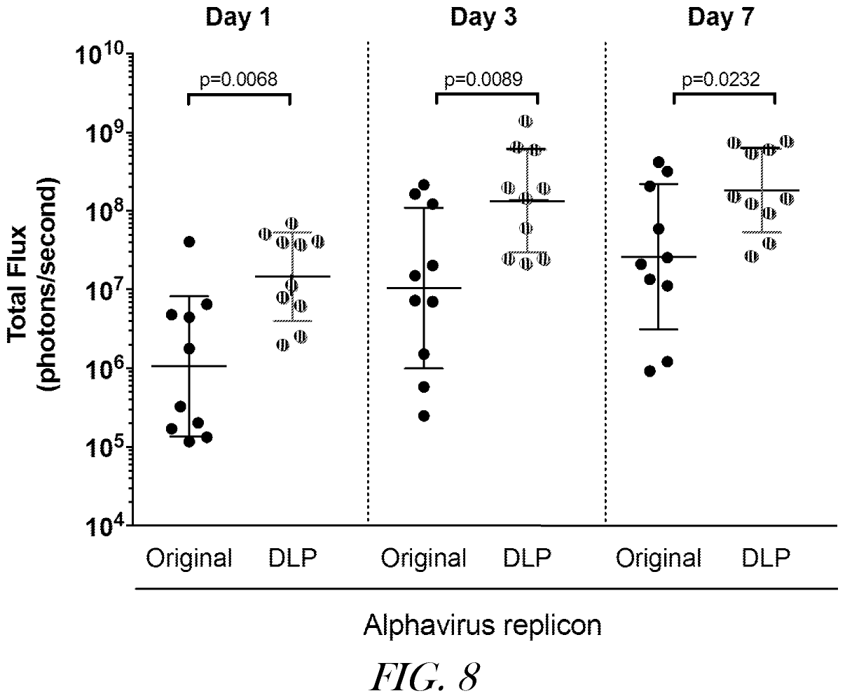

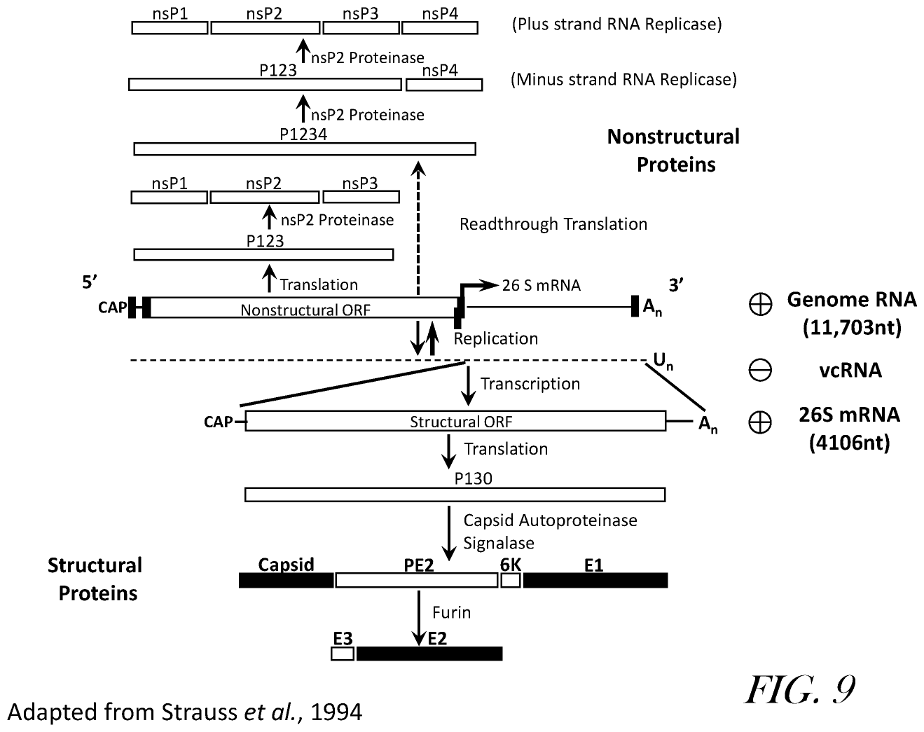

Subsequently, the two RNA replicons alpha-R-rFF and alpha-DLP-2A-nsp-rFF were tested in vivo in Balb/c mice. In this experiment, mice were tested in groups of 10 animals. In these experiments, equal doses of RNA were injected intramuscularly into mice and whole body IVIS (In vivo Imaging System) analysis was carried out over course of one week. Whole body imaging was performed at day 1, day 3 and day 7 post injection. The total flux measured at the injection site is shown in FIG. 8. Although only modest increases in protein expression were noted ex vitro (FIG. 8) from the DLP modified VEEV replicon, statistically significantly higher protein expression was detected at all time points measured from the DLP modified VEEV replicon RNA (FIG. 8). This observation represents a significant advantage, because as unmodified VEEV replicon vectors are capable of very high protein expression that can reach up to 20% of the total cellular protein (Pushko et al 1997). The DLP modified VEEV replicon surpassed even this expression potential and demonstrated superior protein expression; for this reason, the DLP modified alphavirus replicon vector represents a significant advance over existing alphavirus replicon RNA technology.

There are at least three unexpected results that can be drawn from the experimental data presented in the Examples above. First, the DLP motif has been shown to negatively impact translation of mRNAs when a cell is not in an innate immune system activated state. The DLP-containing replicon RNAs disclosed herein were found to have not been negatively impacted in cells at a basal state of innate activation. Second, expression levels, especially for the DLP-containing VEEV replicons, were found to have been even higher than unmodified replicons in vivo; this observation demonstrated that expression levels even from an alphavirus replicon can be increased from previously high historic expression levels. Third, all positive strand RNA viruses have considerable sequence conservation in both the 5′ and 3′ ends of their genomes. The fact that both the VEEV replicon and the EAV replicon are flexible enough to accept incorporation of a stem loop structure (the DLP) in the 5′ end of their RNAs is unexpected.

### Example 6

### In Vivo Immunogenicity Response Using DLP Replicon Expression Systems

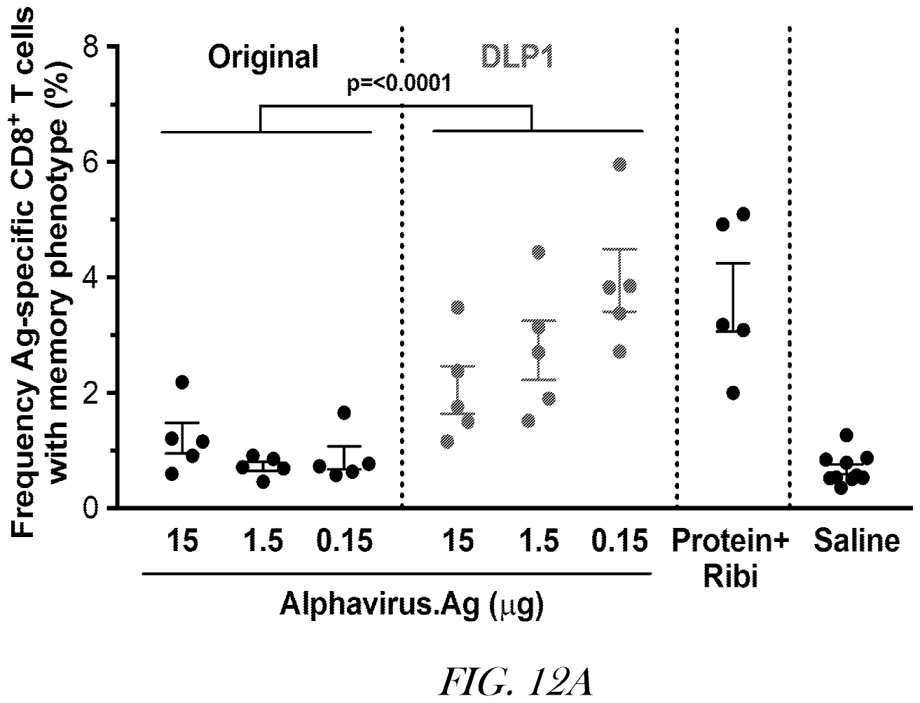

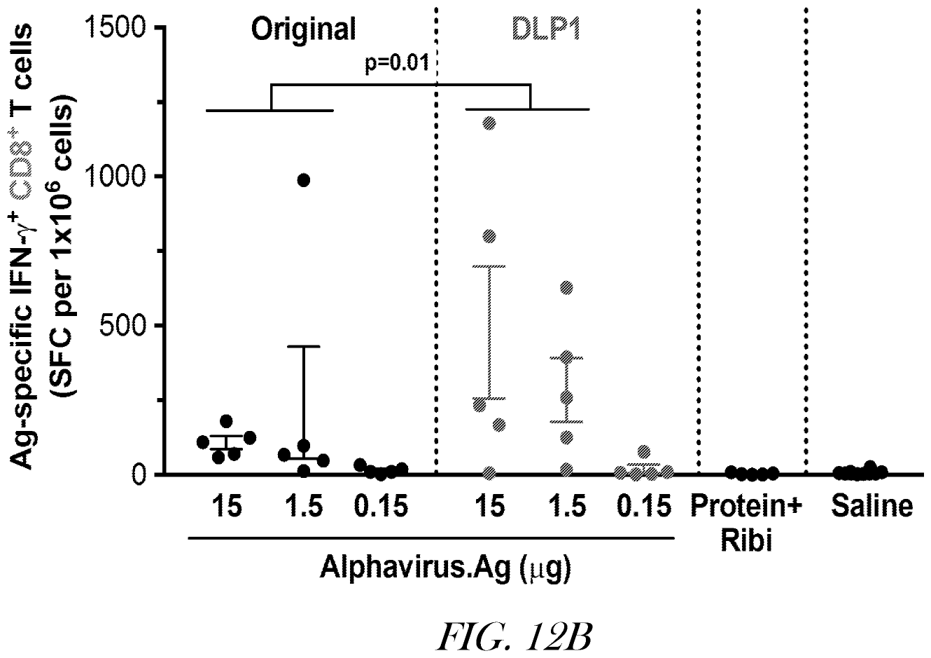

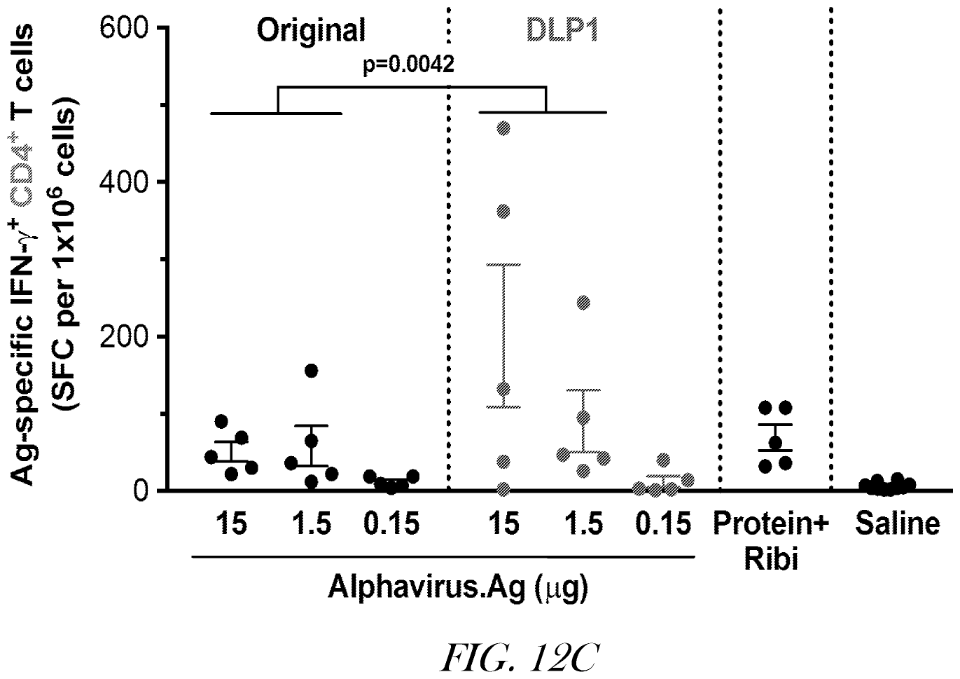

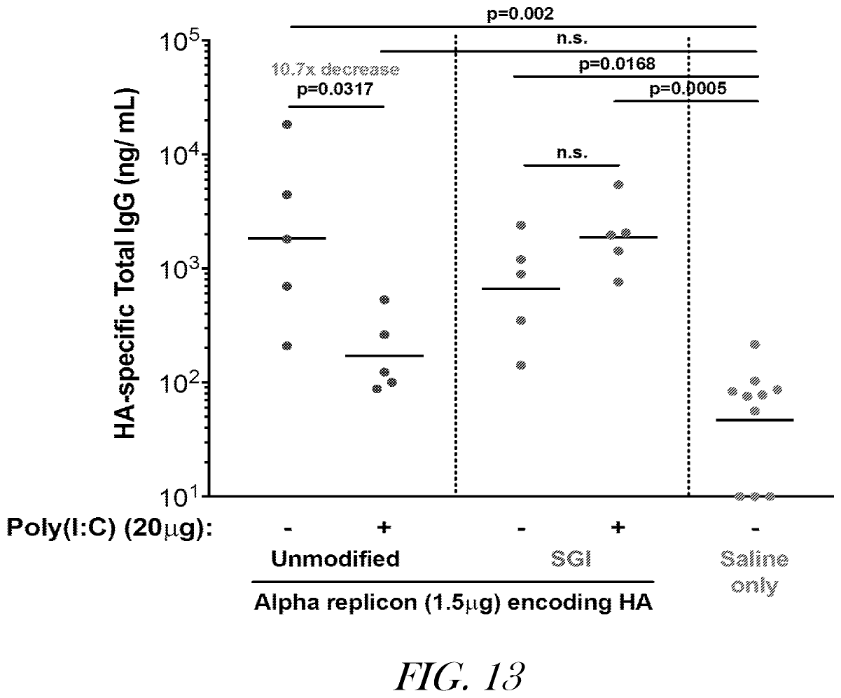

Alphavirus replicon vectors were engineered to contain one or more DLP motifs, as described above. The RNA replicon, Alpha-R-gDLP-HA, containing the DLP sequence was further analyzed in vivo in Balb/c mice. In this experiment, 15 μg, 1.5 μg, or 0.15 μg of RNA encoding Hemagglutinin from Influenza A/Vietnam/1203/2004 (H5N1) was injected into mice at intervals 6 weeks apart. Fourteen days following the final boost, spleens and serum were collected to analyze the immune responses to HA. A summary of the results of these experiments is presented in FIGS. 12A-12C. In FIG. 12A, a significant increase in memory precursor effector cells (MPECs) was observed in constructs containing the DLP motif compared with each comparable dose of an unmodified replicon. HA-specific MPECs were detected using dextramers (H-2 Kd (IYSTVASSL; SEQ ID NO: 44)) along with other population-specific markers (CD8⁺CD44⁺CD62L^{Lo}KLRG-1^{Lo}IL-7Ra^{Hi}CXCR3^{Hi}). Of note, this benefit was also observable at low doses. In FIGS. 12B and 12C, effector T cell responses were measured by the number of antigen-specific HA cells that were secreting IFN-γ following stimulation with a CD4⁺ T cell or CD8⁺ T cell peptide. Animals immunized with replicons containing the DLP motif had a significantly higher frequency of cytokine-expressing CD4⁺ and CD8⁺ T cells are the 15 ug and 1.5 ug doses. Taken together, these data indicate a significant increase in both effector and memory T cell responses in response to immunization with antigen expressed by replicons containing the DLP motif as compared to the unmodified version.

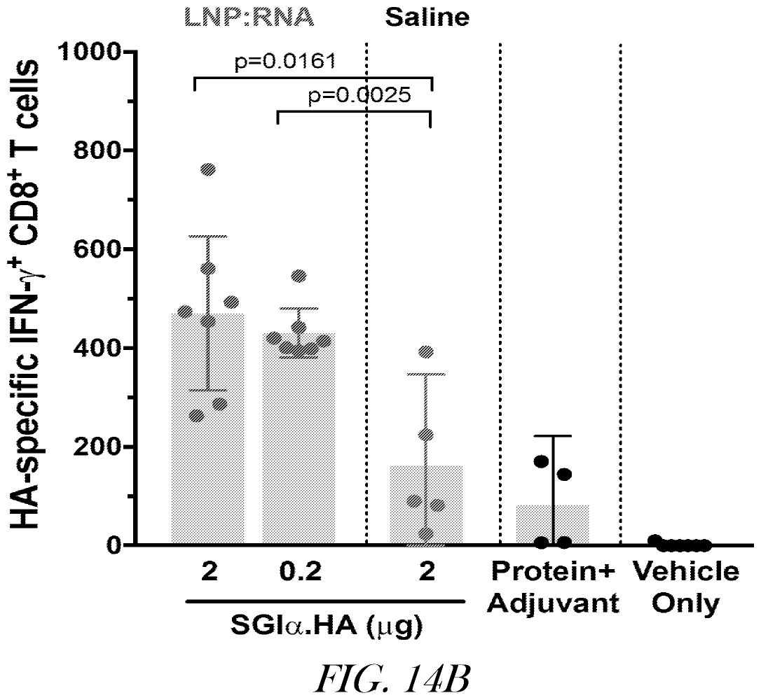

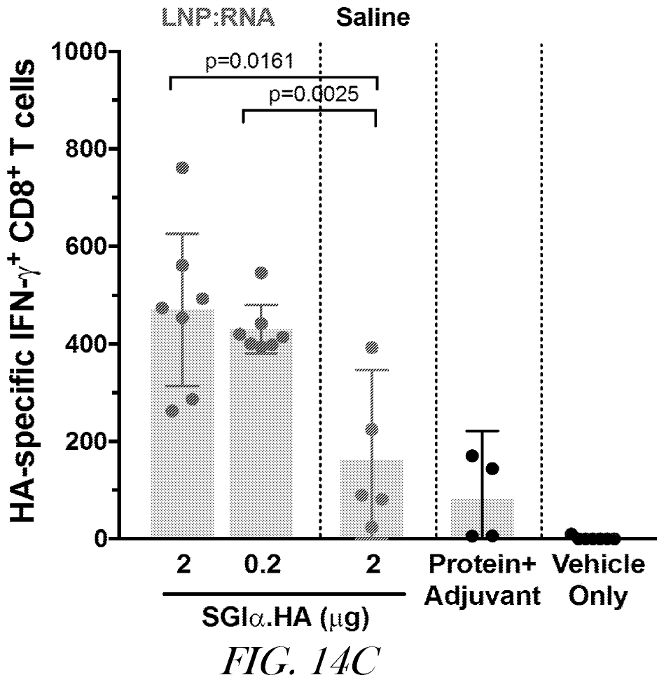

The above DLP-containing replicons were further analyzed in vivo in Balb/c mice for compatibility with LNP formulations. In this experiment, 2 μg or 0.2 μg of RNA encoding Hemagglutinin from Influenza A/Vietnam/1203/2004 (H5N1) was injected into mice at intervals 4 weeks apart. Fourteen days following the final boost, spleens and serum were collected to analyze the immune response to HA. A summary of these experiments is presented in FIG. 14A-14C. In FIGS. 14A-14C, an increase in T-cell and B-cell responses was observed using constructs containing the DLP motif when combined with LNP (cationic lipid nanoparticles) formulations. In FIG. 14A, HA-specific total IgG titers were significantly higher in all dose groups using LNP formulations compared to the group with replicon administered in saline. Furthermore, in FIGS. 14B and 14C, it was observed that HA-specific CD8+ and CD4+ T cells were also significantly higher in all dose groups using LNP formulations compared to the group with replicon administered in saline. Taken together, this data demonstrates that replicon constructs containing the DLP motif are compatible with representative formulations.

### Example 7

### Preventing Suppression of Immune Response Using DLP-Containing Replicons

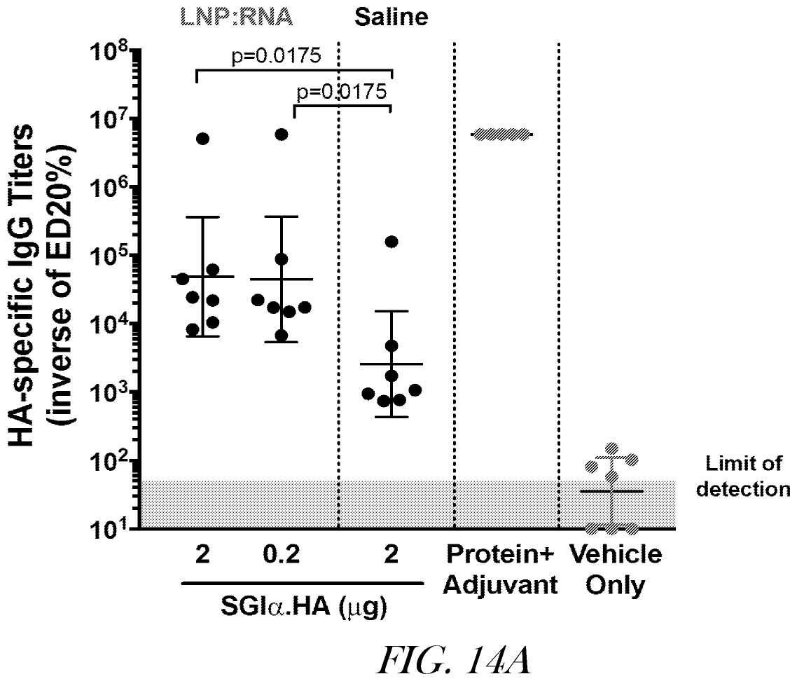

DLP-containing replicons constructed as described above were further evaluated in vivo for the ability to prevent suppression of immune response in Balb/c mice. In these experiments, 1.5 μg of mRNA, with or without DLP motif, and carrying a coding sequence for Hemagglutinin derived from Influenza A/Vietnam/1203/2004 (H5N1) is injected into mice at intervals 4 weeks apart. Approximately 24 hours prior to injection, 6-8 week old BALB/c mice are pre-treated with 20 μg of Poly(I:C) or saline by hydrodynamic tail vein injection to simulate a viral infection. Fourteen days following the final boost, serum from these mice are collected to analyze the immune response to Hemagglutinin (HA). A summary of these experiments is presented in FIG. 13. In FIG. 13, a significant decrease is observed in the serum concentration of HA-specific antibodies in mice who were pre-treated with Poly(I:C) and received a doses of unmodified replicons. The levels in the Poly(I:C) group were not significantly above background. In contrast, animals pre-treated with Poly(I:C) and dosed with a construct containing the DLP motif showed no significant reductions in serum antigen-specific total IgG concentration. Taken together, these data show that the DLP motif protects against suppression of serum antibody levels in response to vaccination following a simulated viral infection compared to the unmodified version.

### Example 8

### Construction of DLP-Containing Expression Cassettes

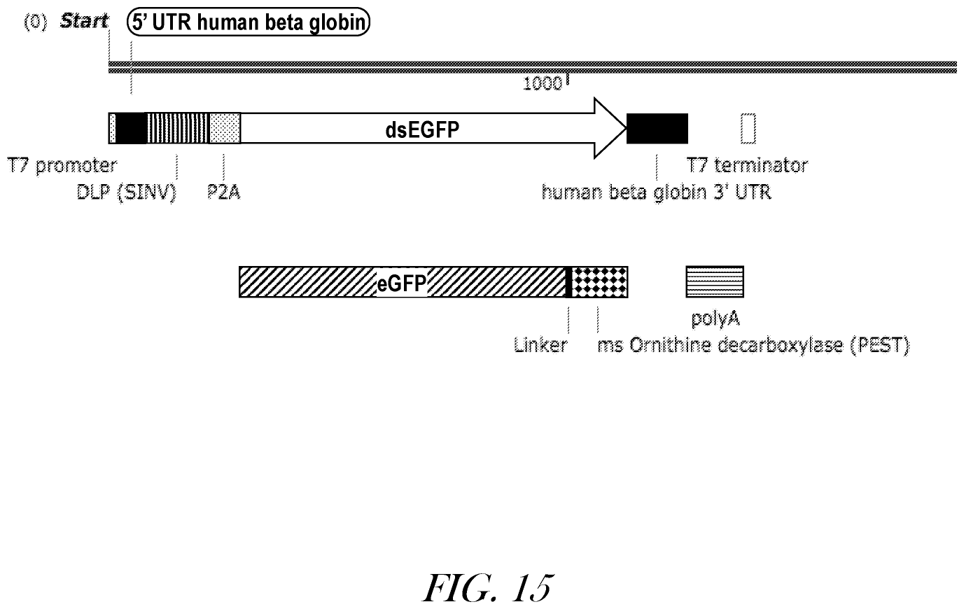

This Example describes the generation of a plasmid vector for ex vitro transcription of an mRNA containing a Sindbis virus DLP element upstream of a gene of interest, e.g., a reporter gene, in accordance with some embodiments of the disclosure. The 5′ and 3′ untranslated regions (UTR) used in these experiments (SEQ ID NO: 36 and SEQ ID NO: 41, respectively) were derived from the human beta globin gene. The 5′ UTR sequence was placed immediately downstream of a T7 promoter (SEQ ID NO: 37) and upstream of the Sindbis virus DLP sequence (SEQ ID NO: 38). In some experiments, the coding sequence for a gene of interest (GOI) was linked to the DLP via a P2A signal, which is an autocatalytic self-cleaving peptide (e.g., autoprotease peptide) derived from the porcine teschovirus-1. In some experiments, a coding sequence for a destabilized form of EGFP reporter gene (dsGFP) which, in this case used as a GOI, was operably linked to the proteolytic PEST degradation signal derived from a mouse ornithine decarboxylase gene (MODC). In some other experiments, a coding sequence of the Red firefly luciferase reported gene was used as the gene of interest (also see, Example 9 below). However, it is contemplated that coding sequences for any gene of interest could be deployed in this configuration. In addition, as illustrated in FIG. 15, a 3′ UTR sequence derived from human beta globin, a polyA tail consisting of 120 adenine residues, and a T7 terminator were inserted downstream and adjacent to the stop codon of dsGFP. The nucleic acid sequences of each of the components described above are as follows:

TABLE 9
Components of DLP dsGFP mRNAs

| | |
|---|---|
| 5′ human beta | 5′-ACATTTGCTTCTGACACAACTGTGTT |
| globin UTR | CACTAGCAACCTCAAACAGACACCGCCGC |
| | CACC-3′ (SEQ ID NO 36) |
| T7 Promoter | 5′-TAATACGACTCACTATAG-3′ |
| | (SEQ ID NO 37) |
| DLP Motif | 5′-ATAGTCAGCATAGTACATTTCATCTG |
| | ACTAATACTACAACACCACCACCATGAAT |
| | AGAGGATTCTTTAACATGCTCGGCCGCCG |
| | CCCCTTCCCGGCCCCCACTGCCATGTGGA |
| | GGCCGCGGAGAAGGAGGCAGGCGGCCCC |
| | G-3′ (SEQ ID NO 38) |
| P2A peptide | 5′-GGAAGCGGAGCTACTAACTTCAGCCT |
| | GCTGAAGCAGGCTGGAGACGTGGAGGAGA |
| | ACCCTGGACCT-3′ (SEQ ID NO 39) |
| DsGFP | 5′-ATGGTGAGCAAGGGCGAGGAGCTGTT |
| | CACCGGGGTGGTGCCCATCCTGGTCGAGC |
| | TGGACGGCGACGTAAACGGCCACAAGTTC |
| | AGCGTGTCCGGCGAGGGCGAGGGCGATGC |
| | CACCTACGGCAAGCTGACCCTGAAGTTCA |
| | TCTGCACCACCGGCAAGCTGCCCGTGCCC |
| | TGGCCCACCCTCGTGACCACCCTGACCTA |
| | CGGCGTGCAGTGCTTCAGCCGCTACCCCG |
| | ACCACATGAAGCAGCACGACTTCTTCAAG |
| | TCCGCCATGCCCGAAGGCTACGTCCAGGA |
| | GCGCACCATCTTCTTCAAGGACGACGGCA |
| | ACTACAAGACCCGCGCCGAGGTGAAGTTC |
| | GAGGGCGACACCCTGGTGAACCGCATCGA |
| | GCTGAAGGGCATCGACTTCAAGGAGGACG |
| | GCAACATCCTGGGGCACAAGCTGGAGTAC |
| | AACTACAACAGCCACAACGTCTATATCAT |
| | GGCCGACAAGCAGAAGAACGGCATCAAGG |
| | TGAACTTCAAGATCCGCCACAACATCGAG |
| | GACGGCAGCGTGCAGCTCGCCGACCACTA |
| | CCAGCAGAACACCCCCATCGGCGACGGCC |
| | CCGTGCTGCTGCCCGACAACCACTACCTG |
| | AGCACCCAGTCCGCCCTGAGCAAAGACCC |
| | CAACGAGAAGCGCGATCACATGGTCCTGC |
| | TGGAGTTCGTGACCGCCGCCGGGATCACT |
| | CTCGGCATGGACGAGCTGTACAAGAAGCT |
| | TAGCCATGGCTTCCCGCCGGAGGTGGAGG |
| | AGCAGGATGATGGCACGCTGCCCATGTCT |
| | TGTGCCCAGGAGAGCGGGATGGACCGTCA |
| | CCCTGCAGCCTGTGCTTCTGCTAGGATCA |
| | ATGTGTAG-3′ (SEQ ID NO 40) |
| 3′ Human beta | 5′-GCTCGCTTTCTTGCTGTCCAATTTCT |
| globin UTR | ATTAAAGGTTCCTTTGTTCCCTAAGTCCA |
| | ACTACTAAACTGGGGGATATTATGAAGGG |
| | CCTTGAGCATCTGGATTCTGCCTAATAAA |
| | AAACATTTATTTTCATTGCAA-3′ |
| | (SEQ ID NO: 41) |
| T7 Terminator | 5′-AACCCCTCTCTAAACGGAGGGGTTTT |
| | TTT-3′ (SEQ ID NO: 42) |
| Sequence of DLP | 5′-TAATACGACTCACTATAGACATTTGC |
| dsGFP Mrna | TTCTGACACAACTGTGTTCACTAGCAACC |
| | TCAAACAGACACCGCCGCCACCATAGTCA |
| | GCATAGTACATTTCATCTGACTAATACTA |
| | CAACACCACCACCATGAATAGAGGATTCT |
| | TTAACATGCTCGGCCGCCGCCCCTTCCCG |
| | GCCCCCACTGCCATGTGGAGGCCGCGGAG |
| | AAGGAGGCAGGCGGCCCCGGGAAGCGGAG |
| | CTACTAACTTCAGCCTGCTGAAGCAGGCT |
| | GGAGACGTGGAGGAGAACCCTGGACCTAT |
| | GGTGAGCAAGGGCGAGGAGCTGTTCACCG |
| | GGGTGGTGCCCATCCTGGTCGAGCTGGAC |
| | GGCGACGTAAACGGCCACAAGTTCAGCGT |
| | GTCCGGCGAGGGCGAGGGCGATGCCACCT |
| | ACGGCAAGCTGACCCTGAAGTTCATCTGC |
| | ACCACCGGCAAGCTGCCCGTGCCCTGGCC |
| | CACCCTCGTGACCACCCTGACCTACGGCG |
| | TGCAGTGCTTCAGCCGCTACCCCGACCAC |
| | ATGAAGCAGCACGACTTCTTCAAGTCCGC |
| | CATGCCCGAAGGCTACGTCCAGGAGCGCA |
| | CCATCTTCTTCAAGGACGACGGCAACTAC |
| | AAGACCCGCGCCGAGGTGAAGTTCGAGGG |
| | CGACACCCTGGTGAACCGCATCGAGCTGA |
| | AGGGCATCGACTTCAAGGAGGACGGCAAC |
| | ATCCTGGGGCACAAGCTGGAGTACAACTA |
| | CAACAGCCACAACGTCTATATCATGGCCG |
| | ACAAGCAGAAGAACGGCATCAAGGTGAAC |
| | TTCAAGATCCGCCACAACATCGAGGACGG |
| | CAGCGTGCAGCTCGCCGACCACTACCAGC |
| | AGAACACCCCCATCGGCGACGGCCCCGTG |
| | CTGCTGCCCGACAACCACTACCTGAGCAC |
| | CCAGTCCGCCCTGAGCAAAGACCCCAACG |
| | AGAAGCGCGATCACATGGTCCTGCTGGAG |
| | TTCGTGACCGCCGCCGGGATCACTCTCGG |
| | CATGGACGAGCTGTACAAGAAGCTTAGCC |
| | ATGGCTTCCCGCCGGAGGTGGAGGAGCAG |
| | GATGATGGCACGCTGCCCATGTCTTGTGC |
| | CCAGGAGAGCGGGATGGACCGTCACCCTG |
| | CAGCCTGTGCTTCTGCTAGGATCAATGTG |
| | TAGGCTCGCTTTCTTGCTGTCCAATTTCT |
| | ATTAAAGGTTCCTTTGTTCCCTAAGTCCA |
| | ACTACTAAACTGGGGGATATTATGAAGGG |
| | CCTTGAGCATCTGGATTCTGCCTAATAAA |
| | AAACATTTATTTTCATTGCAAAAAAAAAA |
| | AAAAAAAAAAAAAAAAAAAAAAAAAAAAA |
| | AAAAAAAAAAAAAAAAAAAAAAAAAAAAA |
| | AAAAAAAAAAAAAAAAAAAAAAAAAAAAA |
| | AAAAAAAAAAAAAAAAAAAAAAAAACCCC |
| | TCTCTAAACGGAGGGGTTTTTTT-3′ |
| | (SEQ ID NO: 43) |

In the above experiments, a DLP sequence from Sindbis virus was used. Additional experiments are performed to incorporate DLP sequences from other Old World alphavirus members such as SV, SFV, BEBV, RRV, SAG, GETV, MIDV, CHIKV, and ONNV, into the nucleic acid molecules of the present disclosure. The linkage of the DLP to the gene of interest can be configured with or without a self-cleaving peptide such as P2A. Without bound to any particular theory, it is believed that the requirement for a 2A sequence or other self-cleaving peptide is dependent on the individual gene being inserted into the gene cassette and on whether the additional amino acids added by the inclusion of DLP would affect the translated proteins function. It is further contemplated that the 5′ and 3′ UTR sequences used here may also be changed for any other set of functional UTRs regardless of origin.

### Example 9

### Ex Vivo Evaluation of Gene Expression in DLP-Containing Expression Cassettes

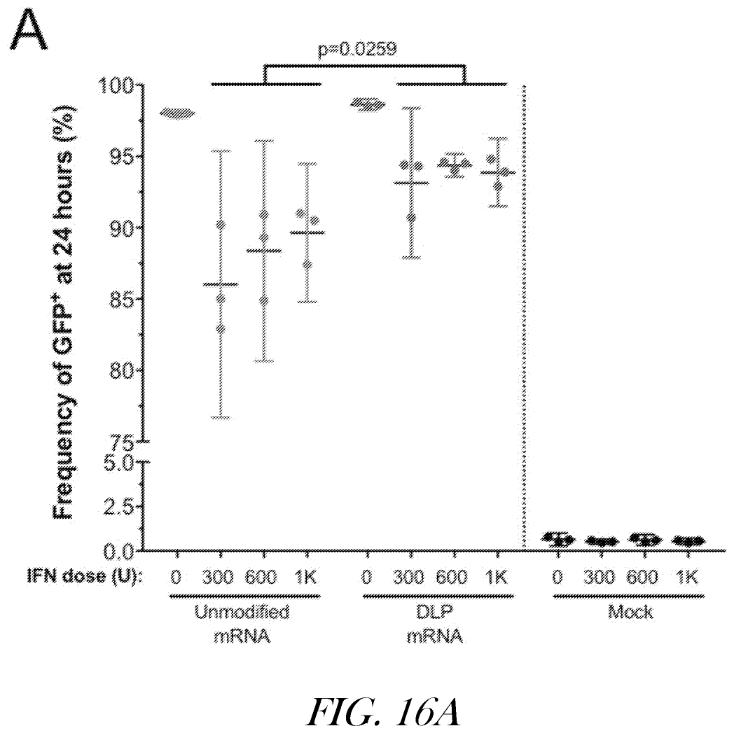

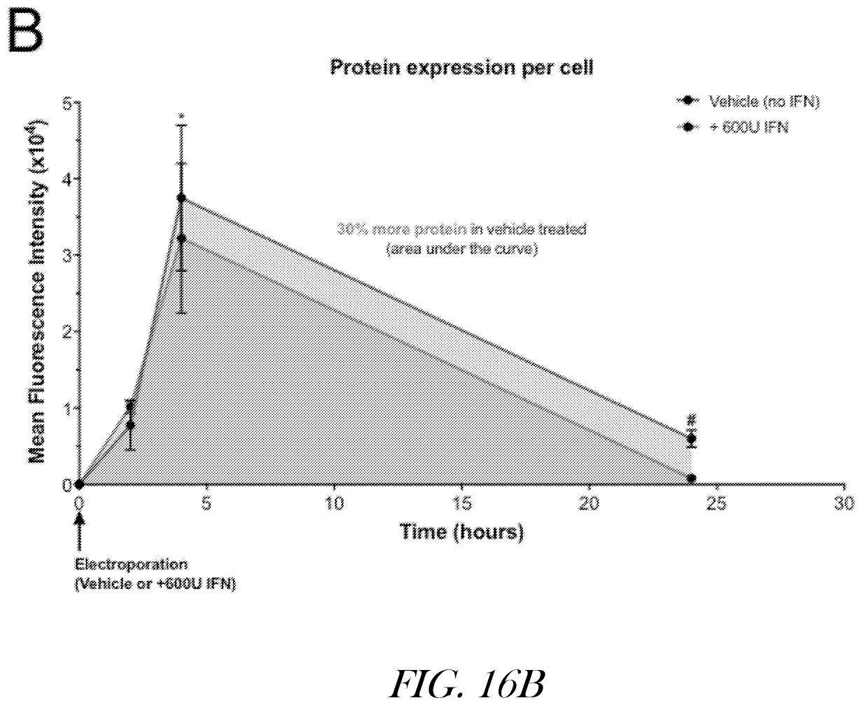

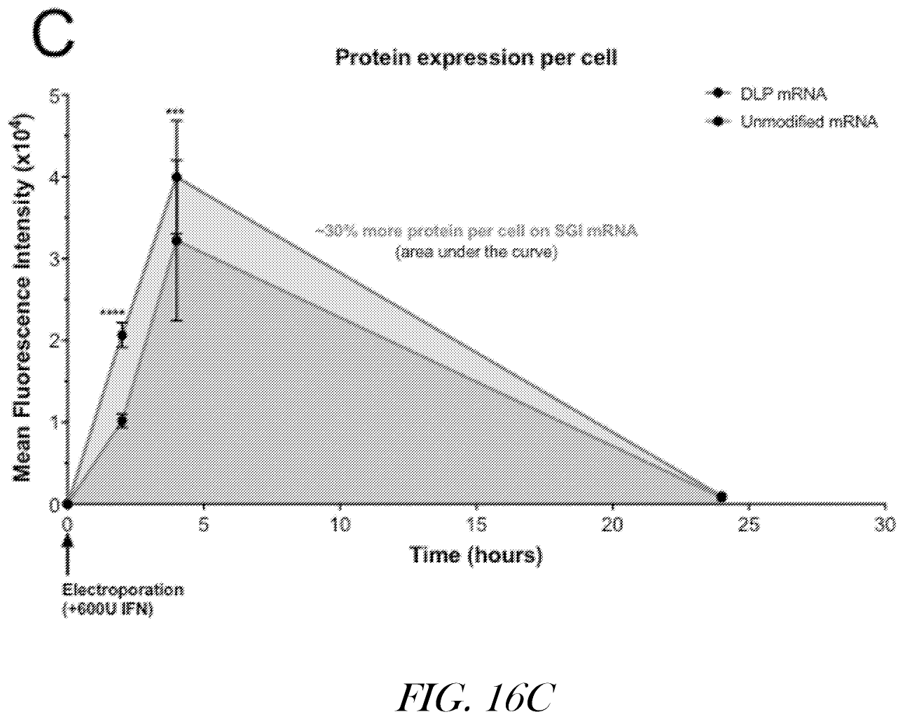

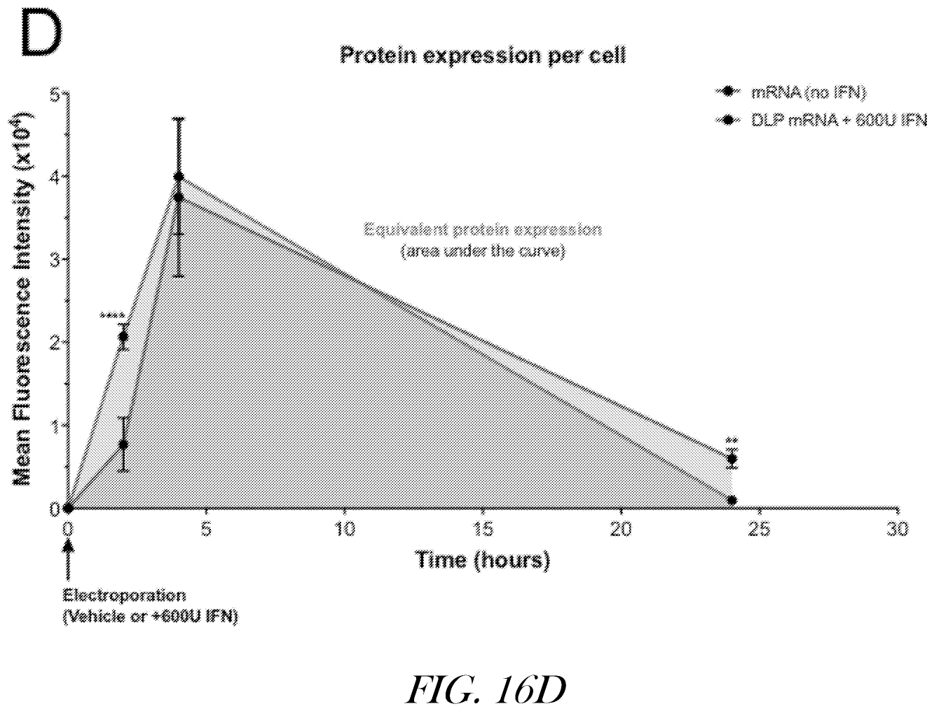

mRNAs derived from DLP-containing expression cassettes engineered to contain one or more DLP motifs, as described above, were evaluated ex vivo for the ability to enhance expression of the gene of interest in BHK-21 cells. As control, mRNA samples lacking the DLP sequence but otherwise identical to the DLP-containing mRNAs described above were assayed in parallel under the same conditions. In these experiments, BHK-21 cells were pre-treated with 300, 600 or 1000 U/mL of universal type I interferon or vehicle control for 2 hours. Following pre-treatment the cells were electroporated, in triplicate, with 2.5 μg of mRNA containing or lacking DLP motifs. The cells were placed back into media containing the same concentrations of interferon used during the pretreatment. The frequency of GFP positive cells and Mean Fluorescence Intensity (MFI) was assayed at 2, 4 and 24 hours post electroporation by flow cytometry. It was observed that DLP-containing mRNA yields significantly higher frequency of GFP positive cells compared to the non-DLP mRNA in the presence of interferon (FIG. 16A).

Furthermore, when the MFI of GFP was normalized to the frequency of GFP positive cells and plotted versus time, it was observed that the unmodified mRNA was sensitive to interferon treatment as exhibited by a statistically significant reduction of 30% in overall protein produced during the 24-hour time course (FIG. 16B). In contrast, the DLP-containing modified mRNA demonstrated resistance to interferon treatment as exhibited by a statistically significant increase of 30% in overall protein production over the control unmodified mRNA during the same 24-hour time course (FIG. 16C). The resistance to interferon treatment conferred by the presence of the DLP motifs was further strengthened by the finding that cells treated with interferon and electroporated with a DLP-containing mRNA produced as much protein as untreated cells electroporated with an unmodified mRNA (FIG. 16C).

### Example 10

### In Vivo Evaluation of Gene Expression in DLP-Containing Expression Cassettes

mRNAs derived from DLP-containing expression cassettes engineered to contain one or more DLP motifs, as described above, are further evaluated in vivo for the ability to enhance expression of the gene of interest in Balb/c mice. In this experiment, 30 μg, 15 μg, or 1.5 μg of DLP-containing mRNA encoding red firefly luciferase is injected into mice at interval of 6 weeks apart. Red firefly luciferase expression is subsequently monitored by IVIS (In vivo Imaging System) analysis at 1, 3, 7, 10, 14, 21 and 28 days post injection. A significant increase in luciferase expression is observed in mice that receive DLP-containing mRNAs when compared to control animals that receive mRNA lacking the DLP motif.

### Example 11

Preventing Suppression of Immune Response Using DLP-Containing mRNAs

DLP-containing mRNAs as described above are further evaluated in vivo for the ability to enhance expression of the gene of interest in Balb/c mice. In this experiment, 30 μg, 15 μg, or 1.5 μg of mRNA, with or without DLP motif, and carrying a coding sequence for Hemagglutinin derived from Influenza A/Vietnam/1203/2004 (H5N1) is injected into mice at intervals 4 weeks apart. Approximately 24 hours prior to injection, mice are pre-treated with 20 μg of Poly(I:C) or saline by hydrodynamic tail vein injection to simulate a viral infection. Fourteen days following the final boost, serum from these mice are collected to analyze the immune response to Hemagglutinin (HA). A significant decrease in the serum concentration of HA-specific antibodies is expected to be observed in mice that are pre-treated with Poly(I:C) and receive a dose of mRNA lacking the DLP sequence. In contrast, animals pre-treated with Poly(I:C) and dosed with mRNA containing the DLP motif are expected to not show significant reductions in serum antigen-specific total IgG concentration.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

All of the references disclosed herein, including but not limited to journal articles, textbooks, publications, patents and patent applications are hereby incorporated by reference in their entireties to the same extent as if each reference was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; any discussion and comment in a specific information source should no way be considered as an admission that such comment was widely accepted as the general opinion in the field.

The discussion of the general compositions and methods given herein is intended for illustrative purposes only. It is not intended to be exhaustive or to limit the disclosure. Individual aspects or features of a particular embodiment are generally not limited to that particular embodiment, but, where applicable, are interchangeable and can be used in a selected embodiment, even if not specifically shown or described. It is expressly contemplated that any aspect or feature of the present disclosure can be combined with any other aspect, features, or combination of aspects and features disclosed herein. Other alternative compositions, methods, and embodiments will be apparent to those of skill in the art upon review of this disclosure, and are to be included within the spirit and purview of this application.

1. A nucleic acid molecule, comprising a nucleic acid sequence encoding a modified viral RNA replicon, wherein the modified viral RNA replicon comprises:
a first nucleic acid sequence encoding a viral capsid enhancer; and
a second nucleic acid downstream from the first nucleic acid sequence, wherein the second nucleic acid sequence is operably linked to the first nucleic acid sequence, wherein the second nucleic acid sequence comprises a sequence encoding a nonstructural protein and a coding sequence for a gene of interest (GOI), wherein the modified viral RNA replicon is derived from an alphavirus or a virus species belonging to the Arterivirus genus of the Arteriviridae family; and the viral capsid enhancer is from an alpha virus.

2. (canceled)

3. The nucleic acid molecule of claim 1, further comprising a promoter operably linked upstream to the first nucleic acid sequence.

4. The nucleic acid molecule of claim 3, further comprising a 5′ UTR sequence operably linked upstream to the first nucleic acid sequence.

5. The nucleic acid molecule of claim 4, wherein the 5′ UTR sequence is operably linked downstream to the promoter and upstream to the first nucleic acid sequence.

6. The nucleic acid molecule of claim 1, further comprising a coding sequence for an autoprotease peptide operably linked operably linked downstream to the first nucleic acid sequence and upstream to the second nucleic acid sequence.

7. (canceled)

8. The nucleic acid molecule of claim 6, wherein the autoprotease peptide comprises a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof.

9. The nucleic acid molecule of claim 1, further comprising a 3′ UTR sequence operably linked downstream to the second sequence nucleic acid sequence.

10-13. (canceled)

14. The nucleic acid molecule of claim 1, wherein the viral capsid enhancer comprises a nucleic acid sequence exhibiting at least 95% sequence identity to at least one of SEQ ID NOs: 1 and 46-52.

15. The nucleic acid molecule of claim 14, wherein the nucleic acid sequence comprises one of SEQ ID NOs: 1 and 46-52.

16. The nucleic acid molecule of any claim 1, wherein the coding sequence for the GOI encodes a polypeptide.

17. The nucleic acid molecule of claim 1, wherein the polypeptide is selected from the group consisting of a therapeutic polypeptide, a prophylactic polypeptide, a diagnostic polypeptide, a nutraceutical polypeptide, an industrial enzyme, a reporter polypeptide, and a combination thereof; or the polypeptide is selected from the group consisting of an antibody, an antigen, an immune modulator, a cytokine, an enzyme, and a combination thereof.

18. (canceled)

19. The nucleic acid molecule of claim 1, further comprising:
a third nucleic acid sequence encoding one or more RNA stem-loops of a second viral capsid enhancer or a variant thereof; and
a fourth nucleic acid sequence operably linked to the third nucleic acid sequence, wherein the fourth nucleic acid sequence comprises a coding sequence for a second gene of interest (GOI).

20. The nucleic acid molecule of claim 19, further comprising a coding sequence for a second autoprotease peptide operably linked downstream to the third nucleic acid sequence and upstream to the fourth nucleic acid sequence.

21. (canceled)

22. The nucleic acid molecule of claim 1, wherein the nucleic acid molecule is an expression vector or a transcription vector.

23-24. (canceled)

25. The nucleic acid molecule of claim 22, wherein the nucleic acid molecule is a plasmid, a bacteriophage vector, a cosmid, a fosmid, a shuttle vector, or a combination thereof.

26-81. (canceled)

82. The nucleic acid molecule of claim 1, wherein the modified viral replicon is derived from an arterivirus virus species selected from the group consisting of Equine arteritis virus (EAV), Porcine respiratory and reproductive syndrome virus (PRRSV), Lactate dehydrogenase elevating virus (LDV), and Simian hemorrhagic fever virus (SHFV).

83. The nucleic acid molecule of claim 82, wherein the second nucleic acid sequence encodes the entire pp1ab nonstructural protein of the modified arterivirus RNA replicon.

84. (canceled)

85. The nucleic acid molecule of claim 83, wherein the modified arterivirus RNA replicon comprises at least two, three, four, five, or six expression cassettes wherein each expression cassette comprises a promoter operably linked to a coding sequence for a gene of interest (GOI).

86. The nucleic acid molecule of claim 85, wherein at least one expression cassette is operably linked downstream of the second nucleic acid sequence encoding the entire pp1ab nonstructural protein of the modified arterivirus RNA replicon.

87. The nucleic acid molecule of claim 83, wherein at least one of the one or more expression cassettes is operably positioned downstream to a transcriptional regulatory sequence (TRS) of the modified arterivirus RNA replicon, wherein the TRS is selected from the group consisting of TRS1, TRS2, TRS3, TRS4, TRS5, TRS6, and TRS7.

88-91. (canceled)

92. The nucleic acid molecule of claim 1, wherein the modified viral RNA replicon comprises a modified RNA replicon derived from an alphavirus virus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), Salmonid alphavirus (SAV), and Buggy Creek virus.

93. The nucleic acid molecule of claim 92, wherein the second nucleic acid sequence encodes nonstructural proteins nsp1-4.

94. The nucleic acid molecule of claim 92, wherein the modified alphavirus RNA replicon is derived from Eastern equine encephalitis virus (EEEV).

95. The nucleic acid molecule of claim 94, wherein the modified alphavirus RNA replicon comprises at least two, three, four, five, or six expression cassettes.

96-163. (canceled)

164. A RNA replicon comprising:
a first nucleic acid sequence encoding a viral capsid enhancer; and
a second nucleic acid downstream from the first nucleic acid sequence, wherein the second nucleic acid sequence is operably linked to the first nucleic acid sequence, wherein the second nucleic acid sequence comprises a sequence encoding a nonstructural protein and a coding sequence for a gene of interest (GOI), wherein the modified viral RNA replicon is derived from an alphavirus or a virus species belonging to the Arterivirus genus of the Arteriviridae family; and the viral capsid enhancer is from an alpha virus.

165. The replicon of claim 164, wherein the viral capsid enhancer comprises a nucleic acid sequence exhibiting at least 95% sequence identity to the RNA version of at least one of SEQ ID NOs: 1 and 46-52.

166. The replicon of claim 165, wherein the nucleic acid sequence comprises the RNA version of any of SEQ ID NOs: 1 and 46-52.

167. The replicon of claim 166, wherein the modified viral RNA replicon is a modified arterivirus RNA replicon derived from an arterivirus virus species selected from the group consisting of Equine arteritis virus (EAV), Porcine respiratory and reproductive syndrome virus (PRRSV), Lactate dehydrogenase elevating virus (LDV), and Simian hemorrhagic fever virus (SHFV).

168. The replicon of claim 167, wherein the second nucleic acid sequence encodes the entire pp1ab nonstructural protein of the modified arterivirus RNA replicon.

169. The replicon of claim 168, wherein the replicon comprises one or more expression cassettes operably positioned downstream to a transcriptional regulatory sequence (TRS) of the modified arterivirus RNA replicon, wherein the TRS is selected from the group consisting of TRS1, TRS2, TRS3, TRS4, TRS5, TRS6, and TRS7.

170. The replicon molecule of claim 164, wherein the modified viral RNA replicon comprises a modified RNA replicon derived from an alphavirus virus species selected from the group consisting of Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), Salmonid alphavirus (SAV), and Buggy Creek virus.

172. The replicon of claim 171, wherein the second nucleic acid sequence encodes nonstructural proteins nsp1-4.

173. The replicon of claim 172, wherein the modified RNA replicon derived is Venezuelan equine encephalitis virus (VEEV).


SEQUENCE LISTING (52 sequences)

SEQ ID NO: 1 - DNA, 145 bp, synthetic construct
  source 1..145: mol_type = other DNA; note = Sindbis virus DNA
atagtcagcatagtacatttcatctgactaatactacaacaccaccaccatgaatagaggattctttaacatgctcggccgccgccccttcccggcccccactgccatgtggaggccgcggagaaggaggcaggcggccccgatg

SEQ ID NO: 2 - DNA, 480 bp, synthetic construct
  misc_feature 1..480: note = Synthetic polynucleotide
  source 1..480: mol_type = other DNA
gctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgcctacgtcgtcgatctctatcaactacccttgcgacttaggcaaccttctccgctactggatttggagggagttttgttagggactggtccctggacttacccgacgcttgtgagcatagtcagcatagtacatttcatctgactaatactacaacaccaccaccatgaatagaggattctttaacatgctcggccgccgccccttcccggcccccactgccatgtggaggccgcggagaaggaggcaggcggccccgatgatggcaaccttctccgctactggatttggagg

SEQ ID NO: 3 - DNA, 66 bp, synthetic construct
  misc_feature 1..66: note = Synthetic polynucleotide
  misc_feature 1..66: note = 2A proteolytic cleavage site
  source 1..66: mol_type = other DNA
ggaagcggagctactaacttcagcctgctgaagcaggctggagacgtggaggagaaccctggacct

SEQ ID NO: 4 - DNA, 1749 bp, synthetic construct
  misc_feature 1..1749: note = Synthetic polynucleotide
  misc_feature 1..1749: note = g-block for construction of rEx-DLP-rFF
  source 1..1749: mol_type = other DNA
cagaggcgcaggacttgatccatggcccacctacagcatgccacctgggccaagaaattgacctttggtccaatgagggcctcgaatattacaaggaagtcaacctgctgtacacacacgtccccatcaaggatggtgtaatacacagttaccctaattgtggccctgcctgtggctgggaaaagcaatccaacaaaatttcgtgcctcccgagagtggcacaaaatttgggctaccactattccccagacttaccaggattttgccccataccaaaagaactcgctgagcattggcccgtagtgtccaatgatagatacccgaattgcttgcaaattaccttacagcaagtatgtgaactcagtaaaccgtgctcagcgggctatatggttggacaatcggttttcgtgcagacgcctggtgtgacatcttactggcttactgaatgggtcgacggcaaagcgcgtgctctaccagattccttattctcgtccggtaggttcgagactaacagccgcgctttcctcgatgaagccgaggaaaagtttgccgccgctcaccctcatgcctgtttgggagaaattaataagtccaccgtgggaggatcccacttcatcttttcccaatatttaccaccattgctacccgcagacgctgttgccctggtaggtgcttcattggctgggaaagctgctaaagctgcttgcagcgttgttgatgtctatgctccatcatttgaaccttatctacaccctgagacactgagtcgcgtgtacaagattatgatcgatttcaagccgtgtaggcttatggtgtggagaaacgcgaccttttatgtccaagagggtgttgatgcagttacatcagcactagcagctgtgtccaaactcatcaaagtgccggccaatgagcctgtttcattccatgtggcatcagggtacagaaccaacgcgctggtagcgccccaggctaaaatttcaattggagcctacgccgccgagtgggcactgtcaactgaaccgccacctgctggttatgcgatcgtgcggcgatatattgtaaagaggctcctcagctcaacagaagtgttcttgtgccgcaggggtgttgtgtcttccacctcagtgcagaccatttgtgcactagagggatgtaaacctctgttcaacttcttacaaattggttcagtcattgggcccgtgtgactctagagtggacctgttcccatcccccgctcaactactcaggtagtggttcgcggcaacgggtacaccgcagttggtaacaagcttgtcgatagtcagcatagtacatttcatctgactaatactacaacaccaccaccatgaatagaggattctttaacatgctcggccgccgccccttcccggcccccactgccatgtggaggccgcggagaaggaggcaggcggccccgatgatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtggg

SEQ ID NO: 5 - DNA, 67 bp, synthetic construct
  misc_feature 1..67: note = Synthetic polynucleotide
  misc_feature 1..67: note = primer pp1a-DLP-F
  misc_feature 1..67: note = RP114
  source 1..67: mol_type = other DNA
gccatgtggaggccgcggagaaggaggcaggcggccccgatgatggcaaccttctccgctactggat

SEQ ID NO: 6 - DNA, 25 bp, synthetic construct
  misc_feature 1..25: note = Synthetic polynucleotide
  misc_feature 1..25: note = primer pBR322-3'SrfI-R
  misc_feature 1..25: note = RP115
  source 1..25: mol_type = other DNA
acaatgttgcctcccacatctgcaa

SEQ ID NO: 7 - DNA, 25 bp, synthetic construct
  misc_feature 1..25: note = Synthetic polynucleotide
  misc_feature 1..25: note = primer pBR322-3'SrfI-F
  misc_feature 1..25: note = RP116
  source 1..25: mol_type = other DNA
gggtcacaaggtagtcgccgtggtt

SEQ ID NO: 8 - DNA, 24 bp, synthetic construct
  misc_feature 1..24: note = Synthetic polynucleotide
  misc_feature 1..24: note = primer pBR322-bla-R
  misc_feature 1..24: note = RP117
  source 1..24: mol_type = other DNA
cgtcaggtggcacttttcggggaa

SEQ ID NO: 9 - DNA, 70 bp, synthetic construct
  misc_feature 1..70: note = Synthetic polynucleotide
  misc_feature 1..70: note = primer pp1a-DLP-2A-F
  source 1..70: mol_type = other DNA
agcctgctgaagcaggctggagacgtggaggagaaccctggacctatggcaaccttctccgctactggat

SEQ ID NO: 10 - DNA, 589 bp, synthetic construct
  misc_feature 1..589: note = Synthetic polynucleotide
  misc_feature 1..589: note = g-block for rEx-DLP-pp1ab-rFF
  source 1..589: mol_type = other DNA
aaacaaataggggttccgcgcacatttccccgaaaagtgccacctgacgtctaagaaaccattattatcatgacattaagcatccgcctttcgttttatttgaccatgttggtatgtaatacgactcactatagctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgcctacgtcgtcgatctctatcaactacccttgcgacttaggcaaccttctccgctactggatttggagggagttttgttagggactggtccctggacttacccgacgcttgtgagcatagtcagcatagtacatttcatctgactaatactacaacaccaccaccatgaatagaggattctttaacatgctcggccgccgccccttcccggcccccactgccatgtggaggccgcggagaaggaggcaggcggccccgatgatggcaac

SEQ ID NO: 11 - DNA, 655 bp, synthetic construct
  misc_feature 1..655: note = Synthetic polynucleotide
  misc_feature 1..655: note = g-block for rEx-DLP-2A-pp1ab-rFF
  source 1..655: mol_type = other DNA
aaacaaataggggttccgcgcacatttccccgaaaagtgccacctgacgtctaagaaaccattattatcatgacattaagcatccgcctttcgttttatttgaccatgttggtatgtaatacgactcactatagctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgcctacgtcgtcgatctctatcaactacccttgcgacttaggcaaccttctccgctactggatttggagggagttttgttagggactggtccctggacttacccgacgcttgtgagcatagtcagcatagtacatttcatctgactaatactacaacaccaccaccatgaatagaggattctttaacatgctcggccgccgccccttcccggcccccactgccatgtggaggccgcggagaaggaggcaggcggccccgatgggaagcggagctactaacttcagcctgctgaagcaggctggagacgtggaggagaaccctggacctatggcaac

SEQ ID NO: 12 - DNA, 655 bp, synthetic construct; identical to SEQ ID NO: 11
  misc_feature 1..655: note = Synthetic polynucleotide
  misc_feature 1..655: note = g-block for rEx-DLP-2A-pp1ab-DLP-rFF
  source 1..655: mol_type = other DNA

SEQ ID NO: 13 - DNA, 30 bp, synthetic construct
  misc_feature 1..30: note = Synthetic polynucleotide
  misc_feature 1..30: note = primer DLP-pp1ab-screen-F
  misc_feature 1..30: note = RP126
  source 1..30: mol_type = other DNA
cagcatcttttactttcaccagcgtttctg

SEQ ID NO: 14 - DNA, 25 bp, synthetic construct
  misc_feature 1..25: note = Synthetic polynucleotide
  misc_feature 1..25: note = primer DLP-pp1ab-screen-R
  misc_feature 1..25: note = RP127
  source 1..25: mol_type = other DNA
ggaactggcgaagccagttttaaca

SEQ ID NO: 15 - DNA, 12529 bp, synthetic construct
  misc_feature 1..12529: note = Synthetic polynucleotide
  misc_feature 1..12529: note = Construct rEx-DLP-rFF
  misc_feature 1..18: note = T7 promoter
  source 1..12529: mol_type = other DNA
taatacgactcactatagctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgccatcgtcgtcgatctctatcaactacccttgcgactatggcaaccttctccgctactggatttggagggagttttgttagggactggtccctggacttacccgacgcttgtgagcatggcgcgggattgtgctgcgaagtggacggctccaccttatgcgccgagtgttttcgcggttgcgaaggaatggagcaatgtcctggcttgttcatgggactgttaaaactggcttcgccagttccagtgggacataagttcctgattggttggtatcgagctgccaaagtcaccgggcgttacaatttccttgagctgttgcaacaccctgctttcgcccagctgcgtgtggttgatgctaggttagccattgaagaggcaagtgtgtttatttccactgaccacgcgtctgctaagcgtttccctggcgctagatttgcgctgacaccggtgtatgctaacgcttgggttgtgagcccggctgctaacagtttgatagtgaccactgaccaggaacaagatgggttctgctggttaaaacttttgccacctgaccgccgtgaggctggtttgcggttgtattacaaccattaccgcgaacaaaggaccgggtggctgtctaaaacaggacttcgcttatggcttggagacctgggtttgggcatcaatgcgagctctggagggctgaaattccacattatgaggggttcgcctcagcgagcttggcatatcacaacacgcagctgcaagctgaagagctactacgtttgtgacatctctgaagcagactggtcctgtttgcctgctggcaactacggcggctacaatccaccaggggacggagcttgcggttacaggtgcttggccttcatgaatggcgccactgttgtgtcggctggttgcagttctgacttgtggtgtgatgatgagttggcttatcgagtctttcaattgtcacccacgttcacggttaccatcccaggtgggcgagtttgtccgaatgccaagtacgcaatgatttgtgacaagcagcactggcgcgtcaaacgtgcaaagggcgtcggcctgtgtctcgatgaaagctgtttcaggggcatctgcaattgccaacgcatgagtggaccaccacctgcacccgtgtcagccgccgtgttagatcacatactggaggcggcgacgtttggcaacgttcgcgtggttacacctgaagggcagccacgccccgtaccagcgccgcgagttcgtcccagcgccaactcttctggagatgtcaaagatccggcgcccgttccgccagtaccaaaaccaaggaccaagcttgccacaccgaacccaactcaggcgcccatcccagcaccgcgcacgcgacttcaaggggcctcaacacaggagccactggcgagtgcaggagttgcttctgactcggcacctaaatggcgtgtggccaaaactgtgtacagctccgcggagcgctttcggaccgaactggtacaacgtgctcggtccgttggggacgttcttgttcaagcgctaccgctcaaaaccccagcagtgcagcggtataccatgactctgaagatgatgcgttcacgcttcagttggcactgcgacgtgtggtaccctttggctgtaatcgcttgtttgctccctatatggccatctcttgctttgctccttagctttgccattgggttgatacccagtgtgggcaataatgttgttctgacagcgcttctggtttcatcagctaattatgttgcgtcaatggaccatcaatgtgaaggtgcggcttgcttagccttgctggaagaagaacactattatagagcggtccgttggcgcccgattacaggcgcgctgtcgcttgtgctcaatttactggggcaggtaggctatgtagctcgttccacctttgatgcagcttatgttccttgcactgtgttcgatctttgcagctttgctattctgtacctctgccgcaatcgttgctggagatgcttcggacgctgtgtgcgagttgggcctgccacgcatgttttgggctccaccgggcaacgagtttccaaactggcgctcattgatttgtgtgaccacttttcaaagcccaccatcgatgttgtgggcatggcaactggttggagcggatgttacacaggaaccgccgcaatggagcgtcagtgtgcctctacggtggaccctcactcgttcgaccagaagaaggcaggagcgactgtttacctcaccccccctgtcaacagcgggtcagcgctgcagtgcctcaatgtcatgtggaagcgaccaattgggtccactgtccttggggaacaaacaggagctgttgtgacggcggtcaagagtatctctttctcacctccctgctgcgtctctaccactttgcccacccgacccggtgtgaccgttgtcgaccatgctctttacaaccggttgactgcttcaggggtcgatcccgctttattgcgtgttgggcaaggtgattttctaaaacttaatccggggttccggctgataggtggatggatttatgggatatgctattttgtgttggtggttgtgtcaacttttacctgcttacctatcaaatgtggcattggcacccgcgaccctttctgccgcagagtgttttctgtacccgtcaccaagacccaagagcactgccatgctggaatgtgtgctagcgctgaaggcatctctctggactctctggggttaactcagttacaaagttactggatcgcagccgtcactagcggattagtgatcttgttggtctgccaccgcctggccatcagcgccttggacttgttgactctagcttcccctttagtgttgcttgtgttcccttgggcatctgtggggcttttacttgcttgcagtctcgctggtgctgctgtgaaaatacagttgttggcgacgctttttgtgaatctattctttccccaagctacccttgtcactatgggatactgggcgtgcgtggcggctttggccgtttacagtttgatgggcttgcgagtgaaagtgaatgtgcccatgtgtgtgacacctgcccattttctgctgctggcgaggtcagctggacagtcaagagagcagatgctccgggtcagcgctgctgcccccaccaattcactgcttggagtggctcgtgattgttatgtcacaggcacaactcggctgtacatacccaaggaaggcgggatggtgtttgaagggctattcaggtcaccgaaggcgcgcggcaacgtcggcttcgtggctggtagcagctacggcacagggtcagtgtggaccaggaacaacgaggtcgtcgtactgacagcgtcacacgtggttggccgcgctaacatggccactctgaagatcggtgacgcaatgctgactctgactttcaaaaagaatggcgacttcgccgaggcagtgacgacacagtccgagctcccaggcaattggccacagttgcatttcgcccaaccaacaaccgggcccgcttcatggtgcactgccacaggagatgaagaaggcttgctcagtggcgaggtttgtctggcgtggactactagtggcgactctggatctgcagtggttcagggtgacgctgtggtaggggtccacaccggttcgaacacaagtggtgttgcctacgtgaccaccccaagcggaaaactccttggcgccgacaccgtgactttgtcatcactgtcaaagcatttcacaggccctttgacatcaatcccgaaggacatccctgacaacattattgccgatgttgatgctgttcctcgttctctggccatgctgattgatggcttatccaatagagagagcagcctttctggacctcagttgttgttaattgcttgttttatgtggtcttatcttaaccaacctgcttacttgccttatgtgctgggcttctttgccgctaacttcttcctgccaaaaagtgttggccgccctgtggtcactgggcttctatggttgtgctgcctcttcacaccgctttccatgcgcttgtgcttgttccatctggtctgtgctaccgtcacgggaaacgtgatatctttgtggttctacatcactgccgctggcacgtcttacctttctgagatgtggttcggaggctatcccaccatgttgtttgtgccacggttcctagtgtaccagttccccggctgggctattggcacagtactagcggtatgcagcatcaccatgctggctgctgccctcggtcacaccctgttactggatgtgttctccgcctcaggtcgctttgacaggactttcatgatgaaatacttcctggagggaggagtgaaagagagtgtcaccgcctcagtcacccgcgcttatggcaaaccaattacccaggagagtctcactgcaacattagctgccctcactgatgatgacttccaattcctctctgatgtgcttgactgtcgggccgtccgatcggcaatgaatctgcgtgccgctctcacaagttttcaagtggcgcagtatcgtaacatccttaatgcatccttgcaagtcgatcgtgacgctgctcgtagtcgcagactaatggcaaaactggctgattttgcggttgaacaagaagtaacagctggagaccgtgttgtggttatcgacggtctggaccgcatggctcacttcaaagacgatttggtgctggttcctttgaccaccaaagtagtaggcggttctaggtgcaccatttgtgacgtcgttaaggaagaagccaatgacaccccagttaagccaatgcccagcaggagacgccgcaagggcctgcctaaaggtgctcagttggagtgggaccgtcaccaggaagagaagaggaacgccggtgatgatgattttgcggtctcgaatgattatgtcaagagagtgccaaagtactgggatcccagcgacacccgaggcacgacagtgaaaatcgccggcactacctatcagaaagtggttgactattcaggcaatgtgcattacgtggagcatcaggaagatctgctagactacgtgctgggcaaggggagctatgaaggcctagatcaggacaaagtgttggacctcacaaacatgcttaaagtggaccccacggagctctcctccaaagacaaagccaaggcgcgtcagcttgctcatctgctgttggatctggctaacccagttgaggcagtgaatcagttaaactgagagcgccccacatctttcccggcgatgtggggcgtcggacctttgctgactctaaagacaagggtttcgtggctctacacagtcgcacaatgtttttagctgcccgggactttttatttaacatcaaatttgtgtgcgacgaagagttcacaaagaccccaaaagacacactgcttgggtacgtacgcgcctgccctggttactggtttattttccgtcgtacgcaccggtcgctgattgatgcatactgggacagtatggagtgcgtttacgcgcttcccaccatatctgattttgatgtgagcccaggtgacgtcgcagtgacgggcgagcgatgggattttgaatctcccggaggaggccgtgcaaaacgtctcacagctgatctggtgcacgcttttcaagggttccacggagcctcttattcctatgatgacaaggtggcagctgctgtcagtggtgacccgtatcggtcggacggcgtcttgtataacacccgttggggcaacattccatattctgtcccaaccaatgctttggaagccacagcttgctaccgtgctggatgtgaggccgttaccgacgggaccaacgtcatcgcaacaattgggcccttcccggagcaacaacccataccggacatcccaaagagcgtgcttgacaactgcgctgacatcagctgtgacgctttcatagcgcccgctgcagagacagccctgtgtggagatttagagaaatacaacctatccacgcagggttttgtgttgcctagtgttttctccatggtgcgggcgtacttaaaagaggagattggagacgctccaccactctacttgccatctactgtaccatctaaaaattcacaagccggaattaacggcgctgagtttcctacaaagtctttacagagctactgtttgattgatgacatggtgtcacagtccatgaaaagcaatctacaaaccgccaccatggcgacttgtaaacggcaatactgttccaaatacaagattaggagcattctgggcaccaacaattacattggcctaggtttgcgtgcctgcctttcgggggttacggccgcattccaaaaagctggaaaggatgggtcaccgatttatttgggcaagtcaaaattcgacccgataccagctcctgacaagtactgccttgaaacagacctggagagttgtgatcgctccaccccggctttggtgcgttggttcgctactaatcttatttttgagctagctggccagcccgagttggtgcacagctacgtgttgaattgctgtcacgatctagttgtggcgggtagtgtagcattcaccaaacgcgggggtttgtcatctggagaccctatcacttccatttccaataccatctattcattggtgctgtacacccagcacatgttgctatgtggacttgaaggctatttcccagagattgcagaaaaatatcttgatggcagcctggagctgcgggacatgttcaagtacgttcgagtgtacatctactcggacgatgtggttctaaccacacccaaccagcattacgcggccagctttgaccgctgggtcccccacctgcaggcgctgctaggtttcaaggttgacccaaagaaaactgtgaacaccagctccccttcctttttgggctgccggttcaagcaagtggacggcaagtgttatctagccagtcttcaggaccgcgttacacgctctctgttataccacattggtgcaaagaatccctcagagtactatgaagctgctgtttccatctttaaggactccattatctgctgtgatgaagactggtggacggacctccatcgacgtatcagtggcgctgcgcgtaccgacggagttgagttccccaccattgaaatgttaacatccttccgcaccaagcagtatgagagtgccgtgtgcacagtttgtggggccgcccccgtggccaagtctgcttgtggagggtggttctgtggcaattgtgtcccgtaccacgcgggtcattgtcacacaacctcgctcttcgccaactgcgggcacgacatcatgtaccgctccacttactgcacaatgtgtgagggttccccaaaacagatggtaccaaaagtgcctcacccgatcctggatcatttgctgtgccacattgattacggcagtaaagaggaactaactctggtagtggcggatggtcgaacaacatcaccgcccgggcgctacaaagtgggtcacaaggtagtcgccgtggttgcagatgtgggaggcaacattgtgtttgggtgcggtcctggatcacacatcgcagtaccacttcaggatacgctcaagggcgtggtggtgaataaagctctgaagaacgccgccgcctctgagtacgtggaaggaccccctgggagtgggaagacttttcacctggtcaaagatgtgctagccgtggtcggtagcgcgaccttggttgtgcccacccacgcgtccatgctggactgcatcaacaagctcaaacaagcgggcgccgatccatactttgtggtgcccaagtatacagttcttgactttccccggcctggcagtggaaacatcacagtgcgactgccacaggtcggaaccagtgagggagaaacctttgtggatgaggtggcctacttctcaccagtggatctggcgcgcattttaacccagggtcgagtcaagggttacggtgatttaaatcagctcgggtgcgtcggacccgcgagcgtgccacgtaacctttggctccgacattttgtcagcctggagcccttgcgagtgtgccatcgattcggcgctgctgtgtgtgatttgatcaagggcatttatccttattatgagccagctccacataccactaaagtggtgtttgtgccaaatccagactttgagaaaggtgtagtcatcaccgcctaccacaaagatcgcggtcttggtcaccgcacaattgattcaattcaaggctgtacattccctgttgtgactcttcgactgcccacaccccaatcactgacgcgcccgcgcgcagttgtggcggttactagggcgtctcaggaattatacatctacgacccctttgatcagcttagcgggttgttgaagttcaccaaggaagcagaggcgcaggacttgatccatggcccacctacagcatgccacctgggccaagaaattgacctttggtccaatgagggcctcgaatattacaaggaagtcaacctgctgtacacacacgtccccatcaaggatggtgtaatacacagttaccctaattgtggccctgcctgtggctgggaaaagcaatccaacaaaatttcgtgcctcccgagagtggcacaaaatttgggctaccactattccccagacttaccaggattttgccccataccaaaagaactcgctgagcattggcccgtagtgtccaatgatagatacccgaattgcttgcaaattaccttacagcaagtatgtgaactcagtaaaccgtgctcagcgggctatatggttggacaatcggttttcgtgcagacgcctggtgtgacatcttactggcttactgaatgggtcgacggcaaagcgcgtgctctaccagattccttattctcgtccggtaggttcgagactaacagccgcgctttcctcgatgaagccgaggaaaagtttgccgccgctcaccctcatgcctgtttgggagaaattaataagtccaccgtgggaggatcccacttcatcttttcccaatatttaccaccattgctacccgcagacgctgttgccctggtaggtgcttcattggctgggaaagctgctaaagctgcttgcagcgttgttgatgtctatgctccatcatttgaaccttatctacaccctgagacactgagtcgcgtgtacaagattatgatcgatttcaagccgtgtaggcttatggtgtggagaaacgcgaccttttatgtccaagagggtgttgatgcagttacatcagcactagcagctgtgtccaaactcatcaaagtgccggccaatgagcctgtttcattccatgtggcatcagggtacagaaccaacgcgctggtagcgccccaggctaaaatttcaattggagcctacgccgccgagtgggcactgtcaactgaaccgccacctgctggttatgcgatcgtgcggcgatatattgtaaagaggctcctcagctcaacagaagtgttcttgtgccgcaggggtgttgtgtcttccacctcagtgcagaccatttgtgcactagagggatgtaaacctctgttcaacttcttacaaattggttcagtcattgggcccgtgtgactctagagtggacctgttcccatcccccgctcaactactcaggtagtggttcgcggcaacgggtacaccgcagttggtaacaagcttgtcgatagtcagcatagtacatttcatctgactaatactacaacaccaccaccatgaatagaggattctttaacatgctcggccgccgccccttcccggcccccactgccatgtggaggccgcggagaaggaggcaggcggccccgatgatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattataactcgagggagccatagattcattttgtggtgacgggattttaggtgagtatctagattactttattctgtccgtcccactcttgctgttgcttactaggtatgtagcatctgggttagtgtatgttttgactgccttgttctattcctttgtattagcagcttatatttggtttgttatagttggaagagccttttctactgcttatgcttttgtgcttttggctgcttttctgttattagtaatgaggatgattgtgggtatgatgcctcgtcttcggtccattttcaaccatcgccaactggtggtagctgattttgtggacacacctagtggacctgttcccatcccccgcccaaccactcaggtagtggttcgcggcaacgggtacaccgcagttggtaacaagcttgtcgatggcgtcaagacgatcacgtccgcaggccgcctcttttcgaaacggacggcggcgacagcctacaagctacaatgacctactgcgcatgtttggtcagatgcgggtccgcaaaccgcccgcgcaacccactcaggctattattgcagagcctggagaccttaggcatgatttaaatcaacaggagcgcgccaccctttcgtcgaacgtacaacggttcttcatgattgggcatggttcactcactgcagatgccggaggactcacgtacaccgtcagttgggttcctaccaaacaaatccagcgcaaagttgcgcctccagcagggccgtaagacgtggatattctcctgtgtggcgtcatgttgaagtagttattagccacccaggaaccaaaaaaaaaaaaaaaaaaaaaaaaaaaaaaaaaaaaaaaaaaaaaaaaaaaaaaaaaaaaaaaaa

SEQ ID NO: 16 - DNA, 12608 bp, synthetic construct
  misc_feature 1..12608: note = Synthetic polynucleotide
  misc_feature 1..12608: note = Construct rEx-DLP-pp1ab-rFF
  misc_feature 1..18: note = T7 promoter
  source 1..12608: mol_type = other DNA
taatacgactcactatagctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgcctacgtcgtcgatctctatcaactacccttgcgacttaggcaaccttctccgctactggatttggagggagttttgttagggactggtccctggacttacccgacgcttgtgagcatagtcagcatagtacatttcatctgactaatactacaacaccaccaccatgaatagaggattctttaacatgctcggccgccgccccttcccggcccccactgccatgtggaggccgcggagaaggaggcaggcggccccgatgatggcaaccttctccgctactggatttggagggagttttgttagggactggtccctggacttacccgacgcttgtgagcatggcgcgggattgtgctgcgaagtggacggctccaccttatgcgccgagtgttttcgcggttgcgaaggaatggagcaatgtcctggcttgttcatgggactgttaaaactggcttcgccagttccagtgggacataagttcctgattggttggtatcgagctgccaaagtcaccgggcgttacaatttccttgagctgttgcaacaccctgctttcgcccagctgcgtgtggttgatgctaggttagccattgaagaggcaagtgtgtttatttccactgaccacgcgtctgctaagcgtttccctggcgctagatttgcgctgacaccggtgtatgctaacgcttgggttgtgagcccggctgctaacagtttgatagtgaccactgaccaggaacaagatgggttctgctggttaaaacttttgccacctgaccgccgtgaggctggtttgcggttgtattacaaccattaccgcgaacaaaggaccgggtggctgtctaaaacaggacttcgcttatggcttggagacctgggtttgggcatcaatgcgagctctggagggctgaaattccacattatgaggggttcgcctcagcgagcttggcatatcacaacacgcagctgcaagctgaagagctactacgtttgtgacatctctgaagcagactggtcctgtttgcctgctggcaactacggcggctacaatccaccaggggacggagcttgcggttacaggtgcttggccttcatgaatggcgccactgttgtgtcggctggttgcagttctgacttgtggtgtgatgatgagttggcttatcgagtctttcaattgtcacccacgttcacggttaccatcccaggtgggcgagtttgtccgaatgccaagtacgcaatgatttgtgacaagcagcactggcgcgtcaaacgtgcaaagggcgtcggcctgtgtctcgatgaaagctgtttcaggggcatctgcaattgccaacgcatgagtggaccaccacctgcacccgtgtcagccgccgtgttagatcacatactggaggcggcgacgtttggcaacgttcgcgtggttacacctgaagggcagccacgccccgtaccagcgccgcgagttcgtcccagcgccaactcttctggagatgtcaaagatccggcgcccgttccgccagtaccaaaaccaaggaccaagcttgccacaccgaacccaactcaggcgcccatcccagcaccgcgcacgcgacttcaaggggcctcaacacaggagccactggcgagtgcaggagttgcttctgactcggcacctaaatggcgtgtggccaaaactgtgtacagctccgcggagcgctttcggaccgaactggtacaacgtgctcggtccgttggggacgttcttgttcaagcgctaccgctcaaaaccccagcagtgcagcggtataccatgactctgaagatgatgcgttcacgcttcagttggcactgcgacgtgtggtaccctttggctgtaatcgcttgtttgctccctatatggccatctcttgctttgctccttagctttgccattgggttgatacccagtgtgggcaataatgttgttctgacagcgcttctggtttcatcagctaattatgttgcgtcaatggaccatcaatgtgaaggtgcggcttgcttagccttgctggaagaagaacactattatagagcggtccgttggcgcccgattacaggcgcgctgtcgcttgtgctcaatttactggggcaggtaggctatgtagctcgttccacctttgatgcagcttatgttccttgcactgtgttcgatctttgcagctttgctattctgtacctctgccgcaatcgttgctggagatgcttcggacgctgtgtgcgagttgggcctgccacgcatgttttgggctccaccgggcaacgagtttccaaactggcgctcattgatttgtgtgaccacttttcaaagcccaccatcgatgttgtgggcatggcaactggttggagcggatgttacacaggaaccgccgcaatggagcgtcagtgtgcctctacggtggaccctcactcgttcgaccagaagaaggcaggagcgactgtttacctcaccccccctgtcaacagcgggtcagcgctgcagtgcctcaatgtcatgtggaagcgaccaattgggtccactgtccttggggaacaaacaggagctgttgtgacggcggtcaagagtatctctttctcacctccctgctgcgtctctaccactttgcccacccgacccggtgtgaccgttgtcgaccatgctctttacaaccggttgactgcttcaggggtcgatcccgctttattgcgtgttgggcaaggtgattttctaaaacttaatccggggttccggctgataggtggatggatttatgggatatgctattttgtgttggtggttgtgtcaacttttacctgcttacctatcaaatgtggcattggcacccgcgaccctttctgccgcagagtgttttctgtacccgtcaccaagacccaagagcactgccatgctggaatgtgtgctagcgctgaaggcatctctctggactctctggggttaactcagttacaaagttactggatcgcagccgtcactagcggattagtgatcttgttggtctgccaccgcctggccatcagcgccttggacttgttgactctagcttcccctttagtgttgcttgtgttcccttgggcatctgtggggcttttacttgcttgcagtctcgctggtgctgctgtgaaaatacagttgttggcgacgctttttgtgaatctattctttccccaagctacccttgtcactatgggatactgggcgtgcgtggcggctttggccgtttacagtttgatgggcttgcgagtgaaagtgaatgtgcccatgtgtgtgacacctgcccattttctgctgctggcgaggtcagctggacagtcaagagagcagatgctccgggtcagcgctgctgcccccaccaattcactgcttggagtggctcgtgattgttatgtcacaggcacaactcggctgtacatacccaaggaaggcgggatggtgtttgaagggctattcaggtcaccgaaggcgcgcggcaacgtcggcttcgtggctggtagcagctacggcacagggtcagtgtggaccaggaacaacgaggtcgtcgtactgacagcgtcacacgtggttggccgcgctaacatggccactctgaagatcggtgacgcaatgctgactctgactttcaaaaagaatggcgacttcgccgaggcagtgacgacacagtccgagctcccaggcaattggccacagttgcatttcgcccaaccaacaaccgggcccgcttcatggtgcactgccacaggagatgaagaaggcttgctcagtggcgaggtttgtctggcgtggactactagtggcgactctggatctgcagtggttcagggtgacgctgtggtaggggtccacaccggttcgaacacaagtggtgttgcctacgtgaccaccccaagcggaaaactccttggcgccgacaccgtgactttgtcatcactgtcaaagcatttcacaggccctttgacatcaatcccgaaggacatccctgacaacattattgccgatgttgatgctgttcctcgttctctggccatgctgattgatggcttatccaatagagagagcagcctttctggacctcagttgttgttaattgcttgttttatgtggtcttatcttaaccaacctgcttacttgccttatgtgctgggcttctttgccgctaacttcttcctgccaaaaagtgttggccgccctgtggtcactgggcttctatggttgtgctgcctcttcacaccgctttccatgcgcttgtgcttgttccatctggtctgtgctaccgtcacgggaaacgtgatatctttgtggttctacatcactgccgctggcacgtcttacctttctgagatgtggttcggaggctatcccaccatgttgtttgtgccacggttcctagtgtaccagttccccggctgggctattggcacagtactagcggtatgcagcatcaccatgctggctgctgccctcggtcacaccctgttactggatgtgttctccgcctcaggtcgctttgacaggactttcatgatgaaatacttcctggagggaggagtgaaagagagtgtcaccgcctcagtcacccgcgcttatggcaaaccaattacccaggagagtctcactgcaacattagctgccctcactgatgatgacttccaattcctctctgatgtgcttgactgtcgggccgtccgatcggcaatgaatctgcgtgccgctctcacaagttttcaagtggcgcagtatcgtaacatccttaatgcatccttgcaagtcgatcgtgacgctgctcgtagtcgcagactaatggcaaaactggctgattttgcggttgaacaagaagtaacagctggagaccgtgttgtggttatcgacggtctggaccgcatggctcacttcaaagacgatttggtgctggttcctttgaccaccaaagtagtaggcggttctaggtgcaccatttgtgacgtcgttaaggaagaagccaatgacaccccagttaagccaatgcccagcaggagacgccgcaagggcctgcctaaaggtgctcagttggagtgggaccgtcaccaggaagagaagaggaacgccggtgatgatgattttgcggtctcgaatgattatgtcaagagagtgccaaagtactgggatcccagcgacacccgaggcacgacagtgaaaatcgccggcactacctatcagaaagtggttgactattcaggcaatgtgcattacgtggagcatcaggaagatctgctagactacgtgctgggcaaggggagctatgaaggcctagatcaggacaaagtgttggacctcacaaacatgcttaaagtggaccccacggagctctcctccaaagacaaagccaaggcgcgtcagcttgctcatctgctgttggatctggctaacccagttgaggcagtgaatcagttaaactgagagcgccccacatctttcccggcgatgtggggcgtcggacctttgctgactctaaagacaagggtttcgtggctctacacagtcgcacaatgtttttagctgcccgggactttttatttaacatcaaatttgtgtgcgacgaagagttcacaaagaccccaaaagacacactgcttgggtacgtacgcgcctgccctggttactggtttattttccgtcgtacgcaccggtcgctgattgatgcatactgggacagtatggagtgcgtttacgcgcttcccaccatatctgattttgatgtgagcccaggtgacgtcgcagtgacgggcgagcgatgggattttgaatctcccggaggaggccgtgcaaaacgtctcacagctgatctggtgcacgcttttcaagggttccacggagcctcttattcctatgatgacaaggtggcagctgctgtcagtggtgacccgtatcggtcggacggcgtcttgtataacacccgttggggcaacattccatattctgtcccaaccaatgctttggaagccacagcttgctaccgtgctggatgtgaggccgttaccgacgggaccaacgtcatcgcaacaattgggcccttcccggagcaacaacccataccggacatcccaaagagcgtgcttgacaactgcgctgacatcagctgtgacgctttcatagcgcccgctgcagagacagccctgtgtggagatttagagaaatacaacctatccacgcagggttttgtgttgcctagtgttttctccatggtgcgggcgtacttaaaagaggagattggagacgctccaccactctacttgccatctactgtaccatctaaaaattcacaagccggaattaacggcgctgagtttcctacaaagtctttacagagctactgtttgattgatgacatggtgtcacagtccatgaaaagcaatctacaaaccgccaccatggcgacttgtaaacggcaatactgttccaaatacaagattaggagcattctgggcaccaacaattacattggcctaggtttgcgtgcctgcctttcgggggttacggccgcattccaaaaagctggaaaggatgggtcaccgatttatttgggcaagtcaaaattcgacccgataccagctcctgacaagtactgccttgaaacagacctggagagttgtgatcgctccaccccggctttggtgcgttggttcgctactaatcttatttttgagctagctggccagcccgagttggtgcacagctacgtgttgaattgctgtcacgatctagttgtggcgggtagtgtagcattcaccaaacgcgggggtttgtcatctggagaccctatcacttccatttccaataccatctattcattggtgctgtacacccagcacatgttgctatgtggacttgaaggctatttcccagagattgcagaaaaatatcttgatggcagcctggagctgcgggacatgttcaagtacgttcgagtgtacatctactcggacgatgtggttctaaccacacccaaccagcattacgcggccagctttgaccgctgggtcccccacctgcaggcgctgctaggtttcaaggttgacccaaagaaaactgtgaacaccagctccccttcctttttgggctgccggttcaagcaagtggacggcaagtgttatctagccagtcttcaggaccgcgttacacgctctctgttataccacattggtgcaaagaatccctcagagtactatgaagctgctgtttccatctttaaggactccattatctgctgtgatgaagactggtggacggacctccatcgacgtatcagtggcgctgcgcgtaccgacggagttgagttccccaccattgaaatgttaacatccttccgcaccaagcagtatgagagtgccgtgtgcacagtttgtggggccgcccccgtggccaagtctgcttgtggagggtggttctgtggcaattgtgtcccgtaccacgcgggtcattgtcacacaacctcgctcttcgccaactgcgggcacgacatcatgtaccgctccacttactgcacaatgtgtgagggttccccaaaacagatggtaccaaaagtgcctcacccgatcctggatcatttgctgtgccacattgattacggcagtaaagaggaactaactctggtagtggcggatggtcgaacaacatcaccgcccgggcgctacaaagtgggtcacaaggtagtcgccgtggttgcagatgtgggaggcaacattgtgtttgggtgcggtcctggatcacacatcgcagtaccacttcaggatacgctcaagggcgtggtggtgaataaagctctgaagaacgccgccgcctctgagtacgtggaaggaccccctgggagtgggaagacttttcacctggtcaaagatgtgctagccgtggtcggtagcgcgaccttggttgtgcccacccacgcgtccatgctggactgcatcaacaagctcaaacaagcgggcgccgatccatactttgtggtgcccaagtatacagttcttgactttccccggcctggcagtggaaacatcacagtgcgactgccacaggtcggaaccagtgagggagaaacctttgtggatgaggtggcctacttctcaccagtggatctggcgcgcattttaacccagggtcgagtcaagggttacggtgatttaaatcagctcgggtgcgtcggacccgcgagcgtgccacgtaacctttggctccgacattttgtcagcctggagcccttgcgagtgtgccatcgattcggcgctgctgtgtgtgatttgatcaagggcatttatccttattatgagccagctccacataccactaaagtggtgtttgtgccaaatccagactttgagaaaggtgtagtcatcaccgcctaccacaaagatcgcggtcttggtcaccgcacaattgattcaattcaaggctgtacattccctgttgtgactcttcgactgcccacaccccaatcactgacgcgcccgcgcgcagttgtggcggttactagggcgtctcaggaattatacatctacgacccctttgatcagcttagcgggttgttgaagttcaccaaggaagcagaggcgcaggacttgatccatggcccacctacagcatgccacctgggccaagaaattgacctttggtccaatgagggcctcgaatattacaaggaagtcaacctgctgtacacacacgtccccatcaaggatggtgtaatacacagttaccctaattgtggccctgcctgtggctgggaaaagcaatccaacaaaatttcgtgcctcccgagagtggcacaaaatttgggctaccactattccccagacttaccaggattttgccccataccaaaagaactcgctgagcattggcccgtagtgtccaatgatagatacccgaattgcttgcaaattaccttacagcaagtatgtgaactcagtaaaccgtgctcagcgggctatatggttggacaatcggttttcgtgcagacgcctggtgtgacatcttactggcttactgaatgggtcgacggcaaagcgcgtgctctaccagattccttattctcgtccggtaggttcgagactaacagccgcgctttcctcgatgaagccgaggaaaagtttgccgccgctcaccctcatgcctgtttgggagaaattaataagtccaccgtgggaggatcccacttcatcttttcccaatatttaccaccattgctacccgcagacgctgttgccctggtaggtgcttcattggctgggaaagctgctaaagctgcttgcagcgttgttgatgtctatgctccatcatttgaaccttatctacaccctgagacactgagtcgcgtgtacaagattatgatcgatttcaagccgtgtaggcttatggtgtggagaaacgcgaccttttatgtccaagagggtgttgatgcagttacatcagcactagcagctgtgtccaaactcatcaaagtgccggccaatgagcctgtttcattccatgtggcatcagggtacagaaccaacgcgctggtagcgccccaggctaaaatttcaattggagcctacgccgccgagtgggcactgtcaactgaaccgccacctgctggttatgcgatcgtgcggcgatatattgtaaagaggctcctcagctcaacagaagtgttcttgtgccgcaggggtgttgtgtcttccacctcagtgcagaccatttgtgcactagagggatgtaaacctctgttcaacttcttacaaattggttcagtcattgggcccgtgtgactctagagtggacctgttcccatcccccgctcaactactcaggtagtggttcgcggcaacgggtacaccgcagttggtaacaagcttgtcgatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattataactcgagggagccatagattcattttgtggtgacgggattttaggtgagtatctagattactttattctgtccgtcccactcttgctgttgcttactaggtatgtagcatctgggttagtgtatgttttgactgccttgttctattcctttgtattagcagcttatatttggtttgttatagttggaagagccttttctactgcttatgcttttgtgcttttggctgcttttctgttattagtaatgaggatgattgtgggtatgatgcctcgtcttcggtccattttcaaccatcgccaactggtggtagctgattttgtggacacacctagtggacctgttcccatcccccgcccaaccactcaggtagtggttcgcggcaacgggtacaccgcagttggtaacaagcttgtcgatggcgtcaagacgatcacgtccgcaggccgcctcttttcgaaacggacggcggcgacagcctacaagctacaatgacctactgcgcatgtttggtcagatgcgggtccgcaaaccgcccgcgcaacccactcaggctattattgcagagcctggagaccttaggcatgatttaaatcaacaggagcgcgccaccctttcgtcgaacgtacaacggttcttcatgattgggcatggttcactcactgcagatgccggaggactcacgtacaccgtcagttgggttcctaccaaacaaatccagcgcaaagttgcgcctccagcagggccgtaagacgtggatattctcctgtgtggcgtcatgttgaagtagttattagccacccaggaaccaaaaaaaaaaaaaaaaaaaaaaaaaaaaaaaaaaaaaaaaaaaaaaaaaaaaaaaaaaaaaaaaa

SEQ ID NO: 17 - DNA, 12674 bp, synthetic construct
  misc_feature 1..12674: note = Synthetic polynucleotide
  misc_feature 1..12674: note = Construct rEx-DLP-2A-pp1ab-rFF
  misc_feature 1..18: note = T7 promoter
  source 1..12674: mol_type = other DNA
taatacgactcactatagctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgcctacgtcgtcgatctctatcaactacccttgcgacttaggcaaccttctccgctactggatttggagggagttttgttagggactggtccctggacttacccgacgcttgtgagcatagtcagcatagtacatttcatctgactaatactacaacaccaccaccatgaatagaggattctttaacatgctcggccgccgccccttcccggcccccactgccatgtggaggccgcggagaaggaggcaggcggccccgatgggaagcggagctactaacttcagcctgctgaagcaggctggagacgtggaggagaaccctggacctatggcaaccttctccgctactggatttggagggagttttgttagggactggtccctggacttacccgacgcttgtgagcatggcgcgggattgtgctgcgaagtggacggctccaccttatgcgccgagtgttttcgcggttgcgaaggaatggagcaatgtcctggcttgttcatgggactgttaaaactggcttcgccagttccagtgggacataagttcctgattggttggtatcgagctgccaaagtcaccgggcgttacaatttccttgagctgttgcaacaccctgctttcgcccagctgcgtgtggttgatgctaggttagccattgaagaggcaagtgtgtttatttccactgaccacgcgtctgctaagcgtttccctggcgctagatttgcgctgacaccggtgtatgctaacgcttgggttgtgagcccggctgctaacagtttgatagtgaccactgaccaggaacaagatgggttctgctggttaaaacttttgccacctgaccgccgtgaggctggtttgcggttgtattacaaccattaccgcgaacaaaggaccgggtggctgtctaaaacaggacttcgcttatggcttggagacctgggtttgggcatcaatgcgagctctggagggctgaaattccacattatgaggggttcgcctcagcgagcttggcatatcacaacacgcagctgcaagctgaagagctactacgtttgtgacatctctgaagcagactggtcctgtttgcctgctggcaactacggcggctacaatccaccaggggacggagcttgcggttacaggtgcttggccttcatgaatggcgccactgttgtgtcggctggttgcagttctgacttgtggtgtgatgatgagttggcttatcgagtctttcaattgtcacccacgttcacggttaccatcccaggtgggcgagtttgtccgaatgccaagtacgcaatgatttgtgacaagcagcactggcgcgtcaaacgtgcaaagggcgtcggcctgtgtctcgatgaaagctgtttcaggggcatctgcaattgccaacgcatgagtggaccaccacctgcacccgtgtcagccgccgtgttagatcacatactggaggcggcgacgtttggcaacgttcgcgtggttacacctgaagggcagccacgccccgtaccagcgccgcgagttcgtcccagcgccaactcttctggagatgtcaaagatccggcgcccgttccgccagtaccaaaaccaaggaccaagcttgccacaccgaacccaactcaggcgcccatcccagcaccgcgcacgcgacttcaaggggcctcaacacaggagccactggcgagtgcaggagttgcttctgactcggcacctaaatggcgtgtggccaaaactgtgtacagctccgcggagcgctttcggaccgaactggtacaacgtgctcggtccgttggggacgttcttgttcaagcgctaccgctcaaaaccccagcagtgcagcggtataccatgactctgaagatgatgcgttcacgcttcagttggcactgcgacgtgtggtaccctttggctgtaatcgcttgtttgctccctatatggccatctcttgctttgctccttagctttgccattgggttgatacccagtgtgggcaataatgttgttctgacagcgcttctggtttcatcagctaattatgttgcgtcaatggaccatcaatgtgaaggtgcggcttgcttagccttgctggaagaagaacactattatagagcggtccgttggcgcccgattacaggcgcgctgtcgcttgtgctcaatttactggggcaggtaggctatgtagctcgttccacctttgatgcagcttatgttccttgcactgtgttcgatctttgcagctttgctattctgtacctctgccgcaatcgttgctggagatgcttcggacgctgtgtgcgagttgggcctgccacgcatgttttgggctccaccgggcaacgagtttccaaactggcgctcattgatttgtgtgaccacttttcaaagcccaccatcgatgttgtgggcatggcaactggttggagcggatgttacacaggaaccgccgcaatggagcgtcagtgtgcctctacggtggaccctcactcgttcgaccagaagaaggcaggagcgactgtttacctcaccccccctgtcaacagcgggtcagcgctgcagtgcctcaatgtcatgtggaagcgaccaattgggtccactgtccttggggaacaaacaggagctgttgtgacggcggtcaagagtatctctttctcacctccctgctgcgtctctaccactttgcccacccgacccggtgtgaccgttgtcgaccatgctctttacaaccggttgactgcttcaggggtcgatcccgctttattgcgtgttgggcaaggtgattttctaaaacttaatccggggttccggctgataggtggatggatttatgggatatgctattttgtgttggtggttgtgtcaacttttacctgcttacctatcaaatgtggcattggcacccgcgaccctttctgccgcagagtgttttctgtacccgtcaccaagacccaagagcactgccatgctggaatgtgtgctagcgctgaaggcatctctctggactctctggggttaactcagttacaaagttactggatcgcagccgtcactagcggattagtgatcttgttggtctgccaccgcctggccatcagcgccttggacttgttgactctagcttcccctttagtgttgcttgtgttcccttgggcatctgtggggcttttacttgcttgcagtctcgctggtgctgctgtgaaaatacagttgttggcgacgctttttgtgaatctattctttccccaagctacccttgtcactatgggatactgggcgtgcgtggcggctttggccgtttacagtttgatgggcttgcgagtgaaagtgaatgtgcccatgtgtgtgacacctgcccattttctgctgctggcgaggtcagctggacagtcaagagagcagatgctccgggtcagcgctgctgcccccaccaattcactgcttggagtggctcgtgattgttatgtcacaggcacaactcggctgtacatacccaaggaaggcgggatggtgtttgaagggctattcaggtcaccgaaggcgcgcggcaacgtcggcttcgtggctggtagcagctacggcacagggtcagtgtggaccaggaacaacgaggtcgtcgtactgacagcgtcacacgtggttggccgcgctaacatggccactctgaagatcggtgacgcaatgctgactctgactttcaaaaagaatggcgacttcgccgaggcagtgacgacacagtccgagctcccaggcaattggccacagttgcatttcgcccaaccaacaaccgggcccgcttcatggtgcactgccacaggagatgaagaaggcttgctcagtggcgaggtttgtctggcgtggactactagtggcgactctggatctgcagtggttcagggtgacgctgtggtaggggtccacaccggttcgaacacaagtggtgttgcctacgtgaccaccccaagcggaaaactccttggcgccgacaccgtgactttgtcatcactgtcaaagcatttcacaggccctttgacatcaatcccgaaggacatccctgacaacattattgccgatgttgatgctgttcctcgttctctggccatgctgattgatggcttatccaatagagagagcagcctttctggacctcagttgttgttaattgcttgttttatgtggtcttatcttaaccaacctgcttacttgccttatgtgctgggcttctttgccgctaacttcttcctgccaaaaagtgttggccgccctgtggtcactgggcttctatggttgtgctgcctcttcacaccgctttccatgcgcttgtgcttgttccatctggtctgtgctaccgtcacgggaaacgtgatatctttgtggttctacatcactgccgctggcacgtcttacctttctgagatgtggttcggaggctatcccaccatgttgtttgtgccacggttcctagtgtaccagttccccggctgggctattggcacagtactagcggtatgcagcatcaccatgctggctgctgccctcggtcacaccctgttactggatgtgttctccgcctcaggtcgctttgacaggactttcatgatgaaatacttcctggagggaggagtgaaagagagtgtcaccgcctcagtcacccgcgcttatggcaaaccaattacccaggagagtctcactgcaacattagctgccctcactgatgatgacttccaattcctctctgatgtgcttgactgtcgggccgtccgatcggcaatgaatctgcgtgccgctctcacaagttttcaagtggcgcagtatcgtaacatccttaatgcatccttgcaagtcgatcgtgacgctgctcgtagtcgcagactaatggcaaaactggctgattttgcggttgaacaagaagtaacagctggagaccgtgttgtggttatcgacggtctggaccgcatggctcacttcaaagacgatttggtgctggttcctttgaccaccaaagtagtaggcggttctaggtgcaccatttgtgacgtcgttaaggaagaagccaatgacaccccagttaagccaatgcccagcaggagacgccgcaagggcctgcctaaaggtgctcagttggagtgggaccgtcaccaggaagagaagaggaacgccggtgatgatgattttgcggtctcgaatgattatgtcaagagagtgccaaagtactgggatcccagcgacacccgaggcacgacagtgaaaatcgccggcactacctatcagaaagtggttgactattcaggcaatgtgcattacgtggagcatcaggaagatctgctagactacgtgctgggcaaggggagctatgaaggcctagatcaggacaaagtgttggacctcacaaacatgcttaaagtggaccccacggagctctcctccaaagacaaagccaaggcgcgtcagcttgctcatctgctgttggatctggctaacccagttgaggcagtgaatcagttaaactgagagcgccccacatctttcccggcgatgtggggcgtcggacctttgctgactctaaagacaagggtttcgtggctctacacagtcgcacaatgtttttagctgcccgggactttttatttaacatcaaatttgtgtgcgacgaagagttcacaaagaccccaaaagacacactgcttgggtacgtacgcgcctgccctggttactggtttattttccgtcgtacgcaccggtcgctgattgatgcatactgggacagtatggagtgcgtttacgcgcttcccaccatatctgattttgatgtgagcccaggtgacgtcgcagtgacgggcgagcgatgggattttgaatctcccggaggaggccgtgcaaaacgtctcacagctgatctggtgcacgcttttcaagggttccacggagcctcttattcctatgatgacaaggtggcagctgctgtcagtggtgacccgtatcggtcggacggcgtcttgtataacacccgttggggcaacattccatattctgtcccaaccaatgctttggaagccacagcttgctaccgtgctggatgtgaggccgttaccgacgggaccaacgtcatcgcaacaattgggcccttcccggagcaacaacccataccggacatcccaaagagcgtgcttgacaactgcgctgacatcagctgtgacgctttcatagcgcccgctgcagagacagccctgtgtggagatttagagaaatacaacctatccacgcagggttttgtgttgcctagtgttttctccatggtgcgggcgtacttaaaagaggagattggagacgctccaccactctacttgccatctactgtaccatctaaaaattcacaagccggaattaacggcgctgagtttcctacaaagtctttacagagctactgtttgattgatgacatggtgtcacagtccatgaaaagcaatctacaaaccgccaccatggcgacttgtaaacggcaatactgttccaaatacaagattaggagcattctgggcaccaacaattacattggcctaggtttgcgtgcctgcctttcgggggttacggccgcattccaaaaagctggaaaggatgggtcaccgatttatttgggcaagtcaaaattcgacccgataccagctcctgacaagtactgccttgaaacagacctggagagttgtgatcgctccaccccggctttggtgcgttggttcgctactaatcttatttttgagctagctggccagcccgagttggtgcacagctacgtgttgaattgctgtcacgatctagttgtggcgggtagtgtagcattcaccaaacgcgggggtttgtcatctggagaccctatcacttccatttccaataccatctattcattggtgctgtacacccagcacatgttgctatgtggacttgaaggctatttcccagagattgcagaaaaatatcttgatggcagcctggagctgcgggacatgttcaagtacgttcgagtgtacatctactcggacgatgtggttctaaccacacccaaccagcattacgcggccagctttgaccgctgggtcccccacctgcaggcgctgctaggtttcaaggttgacccaaagaaaactgtgaacaccagctccccttcctttttgggctgccggttcaagcaagtggacggcaagtgttatctagccagtcttcaggaccgcgttacacgctctctgttataccacattggtgcaaagaatccctcagagtactatgaagctgctgtttccatctttaaggactccattatctgctgtgatgaagactggtggacggacctccatcgacgtatcagtggcgctgcgcgtaccgacggagttgagttccccaccattgaaatgttaacatccttccgcaccaagcagtatgagagtgccgtgtgcacagtttgtggggccgcccccgtggccaagtctgcttgtggagggtggttctgtggcaattgtgtcccgtaccacgcgggtcattgtcacacaacctcgctcttcgccaactgcgggcacgacatcatgtaccgctccacttactgcacaatgtgtgagggttccccaaaacagatggtaccaaaagtgcctcacccgatcctggatcatttgctgtgccacattgattacggcagtaaagaggaactaactctggtagtggcggatggtcgaacaacatcaccgcccgggcgctacaaagtgggtcacaaggtagtcgccgtggttgcagatgtgggaggcaacattgtgtttgggtgcggtcctggatcacacatcgcagtaccacttcaggatacgctcaagggcgtggtggtgaataaagctctgaagaacgccgccgcctctgagtacgtggaaggaccccctgggagtgggaagacttttcacctggtcaaagatgtgctagccgtggtcggtagcgcgaccttggttgtgcccacccacgcgtccatgctggactgcatcaacaagctcaaacaagcgggcgccgatccatactttgtggtgcccaagtatacagttcttgactttccccggcctggcagtggaaacatcacagtgcgactgccacaggtcggaaccagtgagggagaaacctttgtggatgaggtggcctacttctcaccagtggatctggcgcgcattttaacccagggtcgagtcaagggttacggtgatttaaatcagctcgggtgcgtcggacccgcgagcgtgccacgtaacctttggctccgacattttgtcagcctggagcccttgcgagtgtgccatcgattcggcgctgctgtgtgtgatttgatcaagggcatttatccttattatgagccagctccacataccactaaagtggtgtttgtgccaaatccagactttgagaaaggtgtagtcatcaccgcctaccacaaagatcgcggtcttggtcaccgcacaattgattcaattcaaggctgtacattccctgttgtgactcttcgactgcccacaccccaatcactgacgcgcccgcgcgcagttgtggcggttactagggcgtctcaggaattatacatctacgacccctttgatcagcttagcgggttgttgaagttcaccaaggaagcagaggcgcaggacttgatccatggcccacctacagcatgccacctgggccaagaaattgacctttggtccaatgagggcctcgaatattacaaggaagtcaacctgctgtacacacacgtccccatcaaggatggtgtaatacacagttaccctaattgtggccctgcctgtggctgggaaaagcaatccaacaaaatttcgtgcctcccgagagtggcacaaaatttgggctaccactattccccagacttaccaggattttgccccataccaaaagaactcgctgagcattggcccgtagtgtccaatgatagatacccgaattgcttgcaaattaccttacagcaagtatgtgaactcagtaaaccgtgctcagcgggctatatggttggacaatcggttttcgtgcagacgcctggtgtgacatcttactggcttactgaatgggtcgacggcaaagcgcgtgctctaccagattccttattctcgtccggtaggttcgagactaacagccgcgctttcctcgatgaagccgaggaaaagtttgccgccgctcaccctcatgcctgtttgggagaaattaataagtccaccgtgggaggatcccacttcatcttttcccaatatttaccaccattgctacccgcagacgctgttgccctggtaggtgcttcattggctgggaaagctgctaaagctgcttgcagcgttgttgatgtctatgctccatcatttgaaccttatctacaccctgagacactgagtcgcgtgtacaagattatgatcgatttcaagccgtgtaggcttatggtgtggagaaacgcgaccttttatgtccaagagggtgttgatgcagttacatcagcactagcagctgtgtccaaactcatcaaagtgccggccaatgagcctgtttcattccatgtggcatcagggtacagaaccaacgcgctggtagcgccccaggctaaaatttcaattggagcctacgccgccgagtgggcactgtcaactgaaccgccacctgctggttatgcgatcgtgcggcgatatattgtaaagaggctcctcagctcaacagaagtgttcttgtgccgcaggggtgttgtgtcttccacctcagtgcagaccatttgtgcactagagggatgtaaacctctgttcaacttcttacaaattggttcagtcattgggcccgtgtgactctagagtggacctgttcccatcccccgctcaactactcaggtagtggttcgcggcaacgggtacaccgcagttggtaacaagcttgtcgatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattataactcgagggagccatagattcattttgtggtgacgggattttaggtgagtatctagattactttattctgtccgtcccactcttgctgttgcttactaggtatgtagcatctgggttagtgtatgttttgactgccttgttctattcctttgtattagcagcttatatttggtttgttatagttggaagagccttttctactgcttatgcttttgtgcttttggctgcttttctgttattagtaatgaggatgattgtgggtatgatgcctcgtcttcggtccattttcaaccatcgccaactggtggtagctgattttgtggacacacctagtggacctgttcccatcccccgcccaaccactcaggtagtggttcgcggcaacgggtacaccgcagttggtaacaagcttgtcgatggcgtcaagacgatcacgtccgcaggccgcctcttttcgaaacggacggcggcgacagcctacaagctacaatgacctactgcgcatgtttggtcagatgcgggtccgcaaaccgcccgcgcaacccactcaggctattattgcagagcctggagaccttaggcatgatttaaatcaacaggagcgcgccaccctttcgtcgaacgtacaacggttcttcatgattgggcatggttcactcactgcagatgccggaggactcacgtacaccgtcagttgggttcctaccaaacaaatccagcgcaaagttgcgcctccagcagggccgtaagacgtggatattctcctgtgtggcgtcatgttgaagtagttattagccacccaggaaccaaaaaaaaaaaaaaaaaaaaaaaaaaaaaaaaaaaaaaaaaaaaaaaaaaaaaaaaaaaaaaaaa

SEQ ID NO: 18 - DNA, 12819 bp, synthetic construct
  misc_feature 1..12819: note = Synthetic polynucleotide
  misc_feature 1..12819: note = Construct rEx-DLP-2A-pp1ab-DLP-rFF
  misc_feature 1..18: note = T7 promoter
  source 1..12819: mol_type = other DNA
taatacgactcactatagctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgcctacgtcgtcgatctctatcaactacccttgcgacttaggcaaccttctccgctactggatttggagggagttttgttagggactggtccctggacttacccgacgcttgtgagcatagtcagcatagtacatttcatctgactaatactacaacaccaccaccatgaatagaggattctttaacatgctcggccgccgccccttcccggcccccactgccatgtggaggccgcggagaaggaggcaggcggccccgatgggaagcggagctactaacttcagcctgctgaagcaggctggagacgtggaggagaaccctggacctatggcaaccttctccgctactggatttggagggagttttgttagggactggtccctggacttacccgacgcttgtgagcatggcgcgggattgtgctgcgaagtggacggctccaccttatgcgccgagtgttttcgcggttgcgaaggaatggagcaatgtcctggcttgttcatgggactgttaaaactggcttcgccagttccagtgggacataagttcctgattggttggtatcgagctgccaaagtcaccgggcgttacaatttccttgagctgttgcaacaccctgctttcgcccagctgcgtgtggttgatgctaggttagccattgaagaggcaagtgtgtttatttccactgaccacgcgtctgctaagcgtttccctggcgctagatttgcgctgacaccggtgtatgctaacgcttgggttgtgagcccggctgctaacagtttgatagtgaccactgaccaggaacaagatgggttctgctggttaaaacttttgccacctgaccgccgtgaggctggtttgcggttgtattacaaccattaccgcgaacaaaggaccgggtggctgtctaaaacaggacttcgcttatggcttggagacctgggtttgggcatcaatgcgagctctggagggctgaaattccacattatgaggggttcgcctcagcgagcttggcatatcacaacacgcagctgcaagctgaagagctactacgtttgtgacatctctgaagcagactggtcctgtttgcctgctggcaactacggcggctacaatccaccaggggacggagcttgcggttacaggtgcttggccttcatgaatggcgccactgttgtgtcggctggttgcagttctgacttgtggtgtgatgatgagttggcttatcgagtctttcaattgtcacccacgttcacggttaccatcccaggtgggcgagtttgtccgaatgccaagtacgcaatgatttgtgacaagcagcactggcgcgtcaaacgtgcaaagggcgtcggcctgtgtctcgatgaaagctgtttcaggggcatctgcaattgccaacgcatgagtggaccaccacctgcacccgtgtcagccgccgtgttagatcacatactggaggcggcgacgtttggcaacgttcgcgtggttacacctgaagggcagccacgccccgtaccagcgccgcgagttcgtcccagcgccaactcttctggagatgtcaaagatccggcgcccgttccgccagtaccaaaaccaaggaccaagcttgccacaccgaacccaactcaggcgcccatcccagcaccgcgcacgcgacttcaaggggcctcaacacaggagccactggcgagtgcaggagttgcttctgactcggcacctaaatggcgtgtggccaaaactgtgtacagctccgcggagcgctttcggaccgaactggtacaacgtgctcggtccgttggggacgttcttgttcaagcgctaccgctcaaaaccccagcagtgcagcggtataccatgactctgaagatgatgcgttcacgcttcagttggcactgcgacgtgtggtaccctttggctgtaatcgcttgtttgctccctatatggccatctcttgctttgctccttagctttgccattgggttgatacccagtgtgggcaataatgttgttctgacagcgcttctggtttcatcagctaattatgttgcgtcaatggaccatcaatgtgaaggtgcggcttgcttagccttgctggaagaagaacactattatagagcggtccgttggcgcccgattacaggcgcgctgtcgcttgtgctcaatttactggggcaggtaggctatgtagctcgttccacctttgatgcagcttatgttccttgcactgtgttcgatctttgcagctttgctattctgtacctctgccgcaatcgttgctggagatgcttcggacgctgtgtgcgagttgggcctgccacgcatgttttgggctccaccgggcaacgagtttccaaactggcgctcattgatttgtgtgaccacttttcaaagcccaccatcgatgttgtgggcatggcaactggttggagcggatgttacacaggaaccgccgcaatggagcgtcagtgtgcctctacggtggaccctcactcgttcgaccagaagaaggcaggagcgactgtttacctcaccccccctgtcaacagcgggtcagcgctgcagtgcctcaatgtcatgtggaagcgaccaattgggtccactgtccttggggaacaaacaggagctgttgtgacggcggtcaagagtatctctttctcacctccctgctgcgtctctaccactttgcccacccgacccggtgtgaccgttgtcgaccatgctctttacaaccggttgactgcttcaggggtcgatcccgctttattgcgtgttgggcaaggtgattttctaaaacttaatccggggttccggctgataggtggatggatttatgggatatgctattttgtgttggtggttgtgtcaacttttacctgcttacctatcaaatgtggcattggcacccgcgaccctttctgccgcagagtgttttctgtacccgtcaccaagacccaagagcactgccatgctggaatgtgtgctagcgctgaaggcatctctctggactctctggggttaactcagttacaaagttactggatcgcagccgtcactagcggattagtgatcttgttggtctgccaccgcctggccatcagcgccttggacttgttgactctagcttcccctttagtgttgcttgtgttcccttgggcatctgtggggcttttacttgcttgcagtctcgctggtgctgctgtgaaaatacagttgttggcgacgctttttgtgaatctattctttccccaagctacccttgtcactatgggatactgggcgtgcgtggcggctttggccgtttacagtttgatgggcttgcgagtgaaagtgaatgtgcccatgtgtgtgacacctgcccattttctgctgctggcgaggtcagctggacagtcaagagagcagatgctccgggtcagcgctgctgcccccaccaattcactgcttggagtggctcgtgattgttatgtcacaggcacaactcggctgtacatacccaaggaaggcgggatggtgtttgaagggctattcaggtcaccgaaggcgcgcggcaacgtcggcttcgtggctggtagcagctacggcacagggtcagtgtggaccaggaacaacgaggtcgtcgtactgacagcgtcacacgtggttggccgcgctaacatggccactctgaagatcggtgacgcaatgctgactctgactttcaaaaagaatggcgacttcgccgaggcagtgacgacacagtccgagctcccaggcaattggccacagttgcatttcgcccaaccaacaaccgggcccgcttcatggtgcactgccacaggagatgaagaaggcttgctcagtggcgaggtttgtctggcgtggactactagtggcgactctggatctgcagtggttcagggtgacgctgtggtaggggtccacaccggttcgaacacaagtggtgttgcctacgtgaccaccccaagcggaaaactccttggcgccgacaccgtgactttgtcatcactgtcaaagcatttcacaggccctttgacatcaatcccgaaggacatccctgacaacattattgccgatgttgatgctgttcctcgttctctggccatgctgattgatggcttatccaatagagagagcagcctttctggacctcagttgttgttaattgcttgttttatgtggtcttatcttaaccaacctgcttacttgccttatgtgctgggcttctttgccgctaacttcttcctgccaaaaagtgttggccgccctgtggtcactgggcttctatggttgtgctgcctcttcacaccgctttccatgcgcttgtgcttgttccatctggtctgtgctaccgtcacgggaaacgtgatatctttgtggttctacatcactgccgctggcacgtcttacctttctgagatgtggttcggaggctatcccaccatgttgtttgtgccacggttcctagtgtaccagttccccggctgggctattggcacagtactagcggtatgcagcatcaccatgctggctgctgccctcggtcacaccctgttactggatgtgttctccgcctcaggtcgctttgacaggactttcatgatgaaatacttcctggagggaggagtgaaagagagtgtcaccgcctcagtcacccgcgcttatggcaaaccaattacccaggagagtctcactgcaacattagctgccctcactgatgatgacttccaattcctctctgatgtgcttgactgtcgggccgtccgatcggcaatgaatctgcgtgccgctctcacaagttttcaagtggcgcagtatcgtaacatccttaatgcatccttgcaagtcgatcgtgacgctgctcgtagtcgcagactaatggcaaaactggctgattttgcggttgaacaagaagtaacagctggagaccgtgttgtggttatcgacggtctggaccgcatggctcacttcaaagacgatttggtgctggttcctttgaccaccaaagtagtaggcggttctaggtgcaccatttgtgacgtcgttaaggaagaagccaatgacaccccagttaagccaatgcccagcaggagacgccgcaagggcctgcctaaaggtgctcagttggagtgggaccgtcaccaggaagagaagaggaacgccggtgatgatgattttgcggtctcgaatgattatgtcaagagagtgccaaagtactgggatcccagcgacacccgaggcacgacagtgaaaatcgccggcactacctatcagaaagtggttgactattcaggcaatgtgcattacgtggagcatcaggaagatctgctagactacgtgctgggcaaggggagctatgaaggcctagatcaggacaaagtgttggacctcacaaacatgcttaaagtggaccccacggagctctcctccaaagacaaagccaaggcgcgtcagcttgctcatctgctgttggatctggctaacccagttgaggcagtgaatcagttaaactgagagcgccccacatctttcccggcgatgtggggcgtcggacctttgctgactctaaagacaagggtttcgtggctctacacagtcgcacaatgtttttagctgcccgggactttttatttaacatcaaatttgtgtgcgacgaagagttcacaaagaccccaaaagacacactgcttgggtacgtacgcgcctgccctggttactggtttattttccgtcgtacgcaccggtcgctgattgatgcatactgggacagtatggagtgcgtttacgcgcttcccaccatatctgattttgatgtgagcccaggtgacgtcgcagtgacgggcgagcgatgggattttgaatctcccggaggaggccgtgcaaaacgtctcacagctgatctggtgcacgcttttcaagggttccacggagcctcttattcctatgatgacaaggtggcagctgctgtcagtggtgacccgtatcggtcggacggcgtcttgtataacacccgttggggcaacattccatattctgtcccaaccaatgctttggaagccacagcttgctaccgtgctggatgtgaggccgttaccgacgggaccaacgtcatcgcaacaattgggcccttcccggagcaacaacccataccggacatcccaaagagcgtgcttgacaactgcgctgacatcagctgtgacgctttcatagcgcccgctgcagagacagccctgtgtggagatttagagaaatacaacctatccacgcagggttttgtgttgcctagtgttttctccatggtgcgggcgtacttaaaagaggagattggagacgctccaccactctacttgccatctactgtaccatctaaaaattcacaagccggaattaacggcgctgagtttcctacaaagtctttacagagctactgtttgattgatgacatggtgtcacagtccatgaaaagcaatctacaaaccgccaccatggcgacttgtaaacggcaatactgttccaaatacaagattaggagcattctgggcaccaacaattacattggcctaggtttgcgtgcctgcctttcgggggttacggccgcattccaaaaagctggaaaggatgggtcaccgatttatttgggcaagtcaaaattcgacccgataccagctcctgacaagtactgccttgaaacagacctggagagttgtgatcgctccaccccggctttggtgcgttggttcgctactaatcttatttttgagctagctggccagcccgagttggtgcacagctacgtgttgaattgctgtcacgatctagttgtggcgggtagtgtagcattcaccaaacgcgggggtttgtcatctggagaccctatcacttccatttccaataccatctattcattggtgctgtacacccagcacatgttgctatgtggacttgaaggctatttcccagagattgcagaaaaatatcttgatggcagcctggagctgcgggacatgttcaagtacgttcgagtgtacatctactcggacgatgtggttctaaccacacccaaccagcattacgcggccagctttgaccgctgggtcccccacctgcaggcgctgctaggtttcaaggttgacccaaagaaaactgtgaacaccagctccccttcctttttgggctgccggttcaagcaagtggacggcaagtgttatctagccagtcttcaggaccgcgttacacgctctctgttataccacattggtgcaaagaatccctcagagtactatgaagctgctgtttccatctttaaggactccattatctgctgtgatgaagactggtggacggacctccatcgacgtatcagtggcgctgcgcgtaccgacggagttgagttccccaccattgaaatgttaacatccttccgcaccaagcagtatgagagtgccgtgtgcacagtttgtggggccgcccccgtggccaagtctgcttgtggagggtggttctgtggcaattgtgtcccgtaccacgcgggtcattgtcacacaacctcgctcttcgccaactgcgggcacgacatcatgtaccgctccacttactgcacaatgtgtgagggttccccaaaacagatggtaccaaaagtgcctcacccgatcctggatcatttgctgtgccacattgattacggcagtaaagaggaactaactctggtagtggcggatggtcgaacaacatcaccgcccgggcgctacaaagtgggtcacaaggtagtcgccgtggttgcagatgtgggaggcaacattgtgtttgggtgcggtcctggatcacacatcgcagtaccacttcaggatacgctcaagggcgtggtggtgaataaagctctgaagaacgccgccgcctctgagtacgtggaaggaccccctgggagtgggaagacttttcacctggtcaaagatgtgctagccgtggtcggtagcgcgaccttggttgtgcccacccacgcgtccatgctggactgcatcaacaagctcaaacaagcgggcgccgatccatactttgtggtgcccaagtatacagttcttgactttccccggcctggcagtggaaacatcacagtgcgactgccacaggtcggaaccagtgagggagaaacctttgtggatgaggtggcctacttctcaccagtggatctggcgcgcattttaacccagggtcgagtcaagggttacggtgatttaaatcagctcgggtgcgtcggacccgcgagcgtgccacgtaacctttggctccgacattttgtcagcctggagcccttgcgagtgtgccatcgattcggcgctgctgtgtgtgatttgatcaagggcatttatccttattatgagccagctccacataccactaaagtggtgtttgtgccaaatccagactttgagaaaggtgtagtcatcaccgcctaccacaaagatcgcggtcttggtcaccgcacaattgattcaattcaaggctgtacattccctgttgtgactcttcgactgcccacaccccaatcactgacgcgcccgcgcgcagttgtggcggttactagggcgtctcaggaattatacatctacgacccctttgatcagcttagcgggttgttgaagttcaccaaggaagcagaggcgcaggacttgatccatggcccacctacagcatgccacctgggccaagaaattgacctttggtccaatgagggcctcgaatattacaaggaagtcaacctgctgtacacacacgtccccatcaaggatggtgtaatacacagttaccctaattgtggccctgcctgtggctgggaaaagcaatccaacaaaatttcgtgcctcccgagagtggcacaaaatttgggctaccactattccccagacttaccaggattttgccccataccaaaagaactcgctgagcattggcccgtagtgtccaatgatagatacccgaattgcttgcaaattaccttacagcaagtatgtgaactcagtaaaccgtgctcagcgggctatatggttggacaatcggttttcgtgcagacgcctggtgtgacatcttactggcttactgaatgggtcgacggcaaagcgcgtgctctaccagattccttattctcgtccggtaggttcgagactaacagccgcgctttcctcgatgaagccgaggaaaagtttgccgccgctcaccctcatgcctgtttgggagaaattaataagtccaccgtgggaggatcccacttcatcttttcccaatatttaccaccattgctacccgcagacgctgttgccctggtaggtgcttcattggctgggaaagctgctaaagctgcttgcagcgttgttgatgtctatgctccatcatttgaaccttatctacaccctgagacactgagtcgcgtgtacaagattatgatcgatttcaagccgtgtaggcttatggtgtggagaaacgcgaccttttatgtccaagagggtgttgatgcagttacatcagcactagcagctgtgtccaaactcatcaaagtgccggccaatgagcctgtttcattccatgtggcatcagggtacagaaccaacgcgctggtagcgccccaggctaaaatttcaattggagcctacgccgccgagtgggcactgtcaactgaaccgccacctgctggttatgcgatcgtgcggcgatatattgtaaagaggctcctcagctcaacagaagtgttcttgtgccgcaggggtgttgtgtcttccacctcagtgcagaccatttgtgcactagagggatgtaaacctctgttcaacttcttacaaattggttcagtcattgggcccgtgtgactctagagtggacctgttcccatcccccgctcaactactcaggtagtggttcgcggcaacgggtacaccgcagttggtaacaagcttgtcgatagtcagcatagtacatttcatctgactaatactacaacaccaccaccatgaatagaggattctttaacatgctcggccgccgccccttcccggcccccactgccatgtggaggccgcggagaaggaggcaggcggccccgatgatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattataactcgagggagccatagattcattttgtggtgacgggattttaggtgagtatctagattactttattctgtccgtcccactcttgctgttgcttactaggtatgtagcatctgggttagtgtatgttttgactgccttgttctattcctttgtattagcagcttatatttggtttgttatagttggaagagccttttctactgcttatgcttttgtgcttttggctgcttttctgttattagtaatgaggatgattgtgggtatgatgcctcgtcttcggtccattttcaaccatcgccaactggtggtagctgattttgtggacacacctagtggacctgttcccatcccccgcccaaccactcaggtagtggttcgcggcaacgggtacaccgcagttggtaacaagcttgtcgatggcgtcaagacgatcacgtccgcaggccgcctcttttcgaaacggacggcggcgacagcctacaagctacaatgacctactgcgcatgtttggtcagatgcgggtccgcaaaccgcccgcgcaacccactcaggctattattgcagagcctggagaccttaggcatgatttaaatcaacaggagcgcgccaccctttcgtcgaacgtacaacggttcttcatgattgggcatggttcactcactgcagatgccggaggactcacgtacaccgtcagttgggttcctaccaaacaaatccagcgcaaagttgcgcctccagcagggccgtaagacgtggatattctcctgtgtggcgtcatgttgaagtagttattagccacccaggaaccaaaaaaaaaaaaaaaaaaaaaaaaaaaaaaaaaaaaaaaaaaaaaaaaaaaaaaaaaaaaaaaaa

SEQ ID NO: 19 - DNA, 464 bp, synthetic construct
  misc_feature 1..464: note = Synthetic polynucleotide
  misc_feature 1..464: note = Construct Alpha-R-DLP-2A-nsp-rFF
  source 1..464: mol_type = other DNA
gataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggagaaagttcacg

SEQ ID NO: 20 - DNA, 80 bp, synthetic construct
  misc_feature 1..80: note = Synthetic polynucleotide
  misc_feature 1..80: note = primer DLP-rFF-F
  misc_feature 1..80: note = RP112
  source 1..80: mol_type = other DNA
cctgaatggactacgacatagtctagtccgccaagatatcgcaccatagtcagcatagtacatttcatctgactaatact

SEQ ID NO: 21 - DNA, 60 bp, synthetic construct
  misc_feature 1..60: note = Synthetic polynucleotide
  misc_feature 1..60: note = primer DLP-rFF-R
  misc_feature 1..60: note = RP113
  source 1..60: mol_type = other DNA
gcagcttgccaattgctgctgtatcgatcaattaatcacatcttggccacgggtttcttc

SEQ ID NO: 22 - DNA, 67 bp, synthetic construct
  misc_feature 1..67: note = Synthetic polynucleotide
  misc_feature 1..67: note = primer 5'Alpha-P2A-F
  misc_feature 1..67: note = RP124
  source 1..67: mol_type = other DNA
gaagcaggctggagacgtggaggagaaccctggacctgagaaagttcacgttgacatcgaggaagac

SEQ ID NO: 23 - DNA, 30 bp, synthetic construct
  misc_feature 1..30: note = Synthetic polynucleotide
  misc_feature 1..30: note = primer 5'ScaI-R
  misc_feature 1..30: note = RP125
  source 1..30: mol_type = other DNA
caccagtcacagaaaagcatcttacggatg

SEQ ID NO: 24 - DNA, 1014 bp, synthetic construct
  misc_feature 1..1014: note = Synthetic polynucleotide
  misc_feature 1..1014: note = g-block for Alpha-R-DLP-2A-nsp-rFF
  source 1..1014: mol_type = other DNA
tgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaagcatccgcctttcgttttatttgaccatgttggtatgtaatacgactcactatagataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacct

SEQ ID NO: 25 - DNA, 1014 bp, synthetic construct; identical to SEQ ID NO: 24
  misc_feature 1..1014: note = Synthetic polynucleotide
  misc_feature 1..1014: note = g-block for Alpha-R-DLP-2A-nsp-DLP-rFF
  source 1..1014: mol_type = other DNA

SEQ ID NO: 26 - DNA, 9476 bp, synthetic construct
  misc_feature 1..9476: note = Synthetic polynucleotide
  misc_feature 1..9476: note = construct Alpha-R-rFF
  misc_feature 1..18: note = T7 promoter
  source 1..9476: mol_type = other DNA
taatacgactcactatagataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattaattgatcgatacagcagcaattggcaagctgcttacatagaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 27 - DNA, 9621 bp, synthetic construct
  misc_feature 1..9621: note = Synthetic polynucleotide
  misc_feature 1..9621: note = construct Alpha-R-DLP-rFF
  misc_feature 1..18: note = T7 promoter
  source 1..9621: mol_type = other DNA
taatacgactcactatagataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatagtcagcatagtacatttcatctgactaatactacaacaccaccaccatgaatagaggattctttaacatgctcggccgccgccccttcccggcccccactgccatgtggaggccgcggagaaggaggcaggcggccccgatgatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattaattgatcgatacagcagcaattggcaagctgcttacatagaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 28 - DNA, 9876 bp, synthetic construct
  misc_feature 1..9876: note = Synthetic polynucleotide
  misc_feature 1..9876: note = construct Alpha-R-DLP-2A-nsp-rFF
  misc_feature 1..18: note = T7 promoter
  source 1..9876: mol_type = other DNA
taatacgactcactatagataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattaattgatcgatacagcagcaattggcaagctgcttacatagaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 29 - DNA, 10021 bp, synthetic construct
  misc_feature 1..10021: note = Synthetic polynucleotide
  misc_feature 1..10021: note = construct Alpha-R-DLP-2A-nsp-DLP-rFF
  misc_feature 1..18: note = T7 promoter
  source 1..10021: mol_type = other DNA
taatacgactcactatagataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatagtcagcatagtacatttcatctgactaatactacaacaccaccaccatgaatagaggattctttaacatgctcggccgccgccccttcccggcccccactgccatgtggaggccgcggagaaggaggcaggcggccccgatgatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattaattgatcgatacagcagcaattggcaagctgcttacatagaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 30 - DNA, 1150 bp, synthetic construct
  misc_feature 1..1150: note = Synthetic polynucleotide
  misc_feature 1..1150: note = g-block for Alpha-R-DLP-2A-rFF
  source 1..1150: mol_type = other DNA
tctacggctaacctgaatggactacgacatagtctagtccgccaagatatcgcaccatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgcca

SEQ ID NO: 31 - DNA, 1150 bp, synthetic construct; identical to SEQ ID NO: 30
  misc_feature 1..1150: note = Synthetic polynucleotide
  misc_feature 1..1150: note = g-block for Alpha-R-DLP-2A-nsp-DLP-2A-rFF
  source 1..1150: mol_type = other DNA

SEQ ID NO: 32 - DNA, 25 bp, synthetic construct
  misc_feature 1..25: note = Synthetic polynucleotide
  misc_feature 1..25: note = primer Alpha-3'nsp4-F
  misc_feature 1..25: note = RP123
  source 1..25: mol_type = other DNA
ggctgtttaagcttggcaaacctct

SEQ ID NO: 33 - DNA, 23 bp, synthetic construct
  misc_feature 1..23: note = Synthetic polynucleotide
  misc_feature 1..23: note = primer Alpha-3'nsp4-F
  misc_feature 1..23: note = rFF-seq1
  source 1..23: mol_type = other DNA
agcgagaactgcgaggaattctt

SEQ ID NO: 34 - DNA, 12342 bp, synthetic construct
  misc_feature 1..12342: note = Synthetic polypeptide
  misc_feature 1..12342: note = Construct rEx-rFF
  source 1..12342: mol_type = other DNA
gctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgccatcgtcgtcgatctctatcaactacccttgcgactatggcaaccttctccgctactggatttggagggagttttgttagggactggtccctggacttacccgacgcttgtgagcatggcgcgggattgtgctgcgaagtggacggctccaccttatgcgccgagtgttttcgcggttgcgaaggaatggagcaatgtcctggcttgttcatgggactgttaaaactggcttcgccagttccagtgggacataagttcctgattggttggtatcgagctgccaaagtcaccgggcgttacaatttccttgagctgttgcaacaccctgctttcgcccagctgcgtgtggttgatgctaggttagccattgaagaggcaagtgtgtttatttccactgaccacgcgtctgctaagcgtttccctggcgctagatttgcgctgacaccggtgtatgctaacgcttgggttgtgagcccggctgctaacagtttgatagtgaccactgaccaggaacaagatgggttctgctggttaaaacttttgccacctgaccgccgtgaggctggtttgcggttgtattacaaccattaccgcgaacaaaggaccgggtggctgtctaaaacaggacttcgcttatggcttggagacctgggtttgggcatcaatgcgagctctggagggctgaaattccacattatgaggggttcgcctcagcgagcttggcatatcacaacacgcagctgcaagctgaagagctactacgtttgtgacatctctgaagcagactggtcctgtttgcctgctggcaactacggcggctacaatccaccaggggacggagcttgcggttacaggtgcttggccttcatgaatggcgccactgttgtgtcggctggttgcagttctgacttgtggtgtgatgatgagttggcttatcgagtctttcaattgtcacccacgttcacggttaccatcccaggtgggcgagtttgtccgaatgccaagtacgcaatgatttgtgacaagcagcactggcgcgtcaaacgtgcaaagggcgtcggcctgtgtctcgatgaaagctgtttcaggggcatctgcaattgccaacgcatgagtggaccaccacctgcacccgtgtcagccgccgtgttagatcacatactggaggcggcgacgtttggcaacgttcgcgtggttacacctgaagggcagccacgccccgtaccagcgccgcgagttcgtcccagcgccaactcttctggagatgtcaaagatccggcgcccgttccgccagtaccaaaaccaaggaccaagcttgccacaccgaacccaactcaggcgcccatcccagcaccgcgcacgcgacttcaaggggcctcaacacaggagccactggcgagtgcaggagttgcttctgactcggcacctaaatggcgtgtggccaaaactgtgtacagctccgcggagcgctttcggaccgaactggtacaacgtgctcggtccgttggggacgttcttgttcaagcgctaccgctcaaaaccccagcagtgcagcggtataccatgactctgaagatgatgcgttcacgcttcagttggcactgcgacgtgtggtaccctttggctgtaatcgcttgtttgctccctatatggccatctcttgctttgctccttagctttgccattgggttgatacccagtgtgggcaataatgttgttctgacagcgcttctggtttcatcagctaattatgttgcgtcaatggaccatcaatgtgaaggtgcggcttgcttagccttgctggaagaagaacactattatagagcggtccgttggcgcccgattacaggcgcgctgtcgcttgtgctcaatttactggggcaggtaggctatgtagctcgttccacctttgatgcagcttatgttccttgcactgtgttcgatctttgcagctttgctattctgtacctctgccgcaatcgttgctggagatgcttcggacgctgtgtgcgagttgggcctgccacgcatgttttgggctccaccgggcaacgagtttccaaactggcgctcattgatttgtgtgaccacttttcaaagcccaccatcgatgttgtgggcatggcaactggttggagcggatgttacacaggaaccgccgcaatggagcgtcagtgtgcctctacggtggaccctcactcgttcgaccagaagaaggcaggagcgactgtttacctcaccccccctgtcaacagcgggtcagcgctgcagtgcctcaatgtcatgtggaagcgaccaattgggtccactgtccttggggaacaaacaggagctgttgtgacggcggtcaagagtatctctttctcacctccctgctgcgtctctaccactttgcccacccgacccggtgtgaccgttgtcgaccatgctctttacaaccggttgactgcttcaggggtcgatcccgctttattgcgtgttgggcaaggtgattttctaaaacttaatccggggttccggctgataggtggatggatttatgggatatgctattttgtgttggtggttgtgtcaacttttacctgcttacctatcaaatgtggcattggcacccgcgaccctttctgccgcagagtgttttctgtacccgtcaccaagacccaagagcactgccatgctggaatgtgtgctagcgctgaaggcatctctctggactctctggggttaactcagttacaaagttactggatcgcagccgtcactagcggattagtgatcttgttggtctgccaccgcctggccatcagcgccttggacttgttgactctagcttcccctttagtgttgcttgtgttcccttgggcatctgtggggcttttacttgcttgcagtctcgctggtgctgctgtgaaaatacagttgttggcgacgctttttgtgaatctattctttccccaagctacccttgtcactatgggatactgggcgtgcgtggcggctttggccgtttacagtttgatgggcttgcgagtgaaagtgaatgtgcccatgtgtgtgacacctgcccattttctgctgctggcgaggtcagctggacagtcaagagagcagatgctccgggtcagcgctgctgcccccaccaattcactgcttggagtggctcgtgattgttatgtcacaggcacaactcggctgtacatacccaaggaaggcgggatggtgtttgaagggctattcaggtcaccgaaggcgcgcggcaacgtcggcttcgtggctggtagcagctacggcacagggtcagtgtggaccaggaacaacgaggtcgtcgtactgacagcgtcacacgtggttggccgcgctaacatggccactctgaagatcggtgacgcaatgctgactctgactttcaaaaagaatggcgacttcgccgaggcagtgacgacacagtccgagctcccaggcaattggccacagttgcatttcgcccaaccaacaaccgggcccgcttcatggtgcactgccacaggagatgaagaaggcttgctcagtggcgaggtttgtctggcgtggactactagtggcgactctggatctgcagtggttcagggtgacgctgtggtaggggtccacaccggttcgaacacaagtggtgttgcctacgtgaccaccccaagcggaaaactccttggcgccgacaccgtgactttgtcatcactgtcaaagcatttcacaggccctttgacatcaatcccgaaggacatccctgacaacattattgccgatgttgatgctgttcctcgttctctggccatgctgattgatggcttatccaatagagagagcagcctttctggacctcagttgttgttaattgcttgttttatgtggtcttatcttaaccaacctgcttacttgccttatgtgctgggcttctttgccgctaacttcttcctgccaaaaagtgttggccgccctgtggtcactgggcttctatggttgtgctgcctcttcacaccgctttccatgcgcttgtgcttgttccatctggtctgtgctaccgtcacgggaaacgtgatatctttgtggttctacatcactgccgctggcacgtcttacctttctgagatgtggttcggaggctatcccaccatgttgtttgtgccacggttcctagtgtaccagttccccggctgggctattggcacagtactagcggtatgcagcatcaccatgctggctgctgccctcggtcacaccctgttactggatgtgttctccgcctcaggtcgctttgacaggactttcatgatgaaatacttcctggagggaggagtgaaagagagtgtcaccgcctcagtcacccgcgcttatggcaaaccaattacccaggagagtctcactgcaacattagctgccctcactgatgatgacttccaattcctctctgatgtgcttgactgtcgggccgtccgatcggcaatgaatctgcgtgccgctctcacaagttttcaagtggcgcagtatcgtaacatccttaatgcatccttgcaagtcgatcgtgacgctgctcgtagtcgcagactaatggcaaaactggctgattttgcggttgaacaagaagtaacagctggagaccgtgttgtggttatcgacggtctggaccgcatggctcacttcaaagacgatttggtgctggttcctttgaccaccaaagtagtaggcggttctaggtgcaccatttgtgacgtcgttaaggaagaagccaatgacaccccagttaagccaatgcccagcaggagacgccgcaagggcctgcctaaaggtgctcagttggagtgggaccgtcaccaggaagagaagaggaacgccggtgatgatgattttgcggtctcgaatgattatgtcaagagagtgccaaagtactgggatcccagcgacacccgaggcacgacagtgaaaatcgccggcactacctatcagaaagtggttgactattcaggcaatgtgcattacgtggagcatcaggaagatctgctagactacgtgctgggcaaggggagctatgaaggcctagatcaggacaaagtgttggacctcacaaacatgcttaaagtggaccccacggagctctcctccaaagacaaagccaaggcgcgtcagcttgctcatctgctgttggatctggctaacccagttgaggcagtgaatcagttaaactgagagcgccccacatctttcccggcgatgtggggcgtcggacctttgctgactctaaagacaagggtttcgtggctctacacagtcgcacaatgtttttagctgcccgggactttttatttaacatcaaatttgtgtgcgacgaagagttcacaaagaccccaaaagacacactgcttgggtacgtacgcgcctgccctggttactggtttattttccgtcgtacgcaccggtcgctgattgatgcatactgggacagtatggagtgcgtttacgcgcttcccaccatatctgattttgatgtgagcccaggtgacgtcgcagtgacgggcgagcgatgggattttgaatctcccggaggaggccgtgcaaaacgtctcacagctgatctggtgcacgcttttcaagggttccacggagcctcttattcctatgatgacaaggtggcagctgctgtcagtggtgacccgtatcggtcggacggcgtcttgtataacacccgttggggcaacattccatattctgtcccaaccaatgctttggaagccacagcttgctaccgtgctggatgtgaggccgttaccgacgggaccaacgtcatcgcaacaattgggcccttcccggagcaacaacccataccggacatcccaaagagcgtgcttgacaactgcgctgacatcagctgtgacgctttcatagcgcccgctgcagagacagccctgtgtggagatttagagaaatacaacctatccacgcagggttttgtgttgcctagtgttttctccatggtgcgggcgtacttaaaagaggagattggagacgctccaccactctacttgccatctactgtaccatctaaaaattcacaagccggaattaacggcgctgagtttcctacaaagtctttacagagctactgtttgattgatgacatggtgtcacagtccatgaaaagcaatctacaaaccgccaccatggcgacttgtaaacggcaatactgttccaaatacaagattaggagcattctgggcaccaacaattacattggcctaggtttgcgtgcctgcctttcgggggttacggccgcattccaaaaagctggaaaggatgggtcaccgatttatttgggcaagtcaaaattcgacccgataccagctcctgacaagtactgccttgaaacagacctggagagttgtgatcgctccaccccggctttggtgcgttggttcgctactaatcttatttttgagctagctggccagcccgagttggtgcacagctacgtgttgaattgctgtcacgatctagttgtggcgggtagtgtagcattcaccaaacgcgggggtttgtcatctggagaccctatcacttccatttccaataccatctattcattggtgctgtacacccagcacatgttgctatgtggacttgaaggctatttcccagagattgcagaaaaatatcttgatggcagcctggagctgcgggacatgttcaagtacgttcgagtgtacatctactcggacgatgtggttctaaccacacccaaccagcattacgcggccagctttgaccgctgggtcccccacctgcaggcgctgctaggtttcaaggttgacccaaagaaaactgtgaacaccagctccccttcctttttgggctgccggttcaagcaagtggacggcaagtgttatctagccagtcttcaggaccgcgttacacgctctctgttataccacattggtgcaaagaatccctcagagtactatgaagctgctgtttccatctttaaggactccattatctgctgtgatgaagactggtggacggacctccatcgacgtatcagtggcgctgcgcgtaccgacggagttgagttccccaccattgaaatgttaacatccttccgcaccaagcagtatgagagtgccgtgtgcacagtttgtggggccgcccccgtggccaagtctgcttgtggagggtggttctgtggcaattgtgtcccgtaccacgcgggtcattgtcacacaacctcgctcttcgccaactgcgggcacgacatcatgtaccgctccacttactgcacaatgtgtgagggttccccaaaacagatggtaccaaaagtgcctcacccgatcctggatcatttgctgtgccacattgattacggcagtaaagaggaactaactctggtagtggcggatggtcgaacaacatcaccgcccgggcgctacaaagtgggtcacaaggtagtcgccgtggttgcagatgtgggaggcaacattgtgtttgggtgcggtcctggatcacacatcgcagtaccacttcaggatacgctcaagggcgtggtggtgaataaagctctgaagaacgccgccgcctctgagtacgtggaaggaccccctgggagtgggaagacttttcacctggtcaaagatgtgctagccgtggtcggtagcgcgaccttggttgtgcccacccacgcgtccatgctggactgcatcaacaagctcaaacaagcgggcgccgatccatactttgtggtgcccaagtatacagttcttgactttccccggcctggcagtggaaacatcacagtgcgactgccacaggtcggaaccagtgagggagaaacctttgtggatgaggtggcctacttctcaccagtggatctggcgcgcattttaacccagggtcgagtcaagggttacggtgatttaaatcagctcgggtgcgtcggacccgcgagcgtgccacgtaacctttggctccgacattttgtcagcctggagcccttgcgagtgtgccatcgattcggcgctgctgtgtgtgatttgatcaagggcatttatccttattatgagccagctccacataccactaaagtggtgtttgtgccaaatccagactttgagaaaggtgtagtcatcaccgcctaccacaaagatcgcggtcttggtcaccgcacaattgattcaattcaaggctgtacattccctgttgtgactcttcgactgcccacaccccaatcactgacgcgcccgcgcgcagttgtggcggttactagggcgtctcaggaattatacatctacgacccctttgatcagcttagcgggttgttgaagttcaccaaggaagcagaggcgcaggacttgatccatggcccacctacagcatgccacctgggccaagaaattgacctttggtccaatgagggcctcgaatattacaaggaagtcaacctgctgtacacacacgtccccatcaaggatggtgtaatacacagttaccctaattgtggccctgcctgtggctgggaaaagcaatccaacaaaatttcgtgcctcccgagagtggcacaaaatttgggctaccactattccccagacttaccaggattttgccccataccaaaagaactcgctgagcattggcccgtagtgtccaatgatagatacccgaattgcttgcaaattaccttacagcaagtatgtgaactcagtaaaccgtgctcagcgggctatatggttggacaatcggttttcgtgcagacgcctggtgtgacatcttactggcttactgaatgggtcgacggcaaagcgcgtgctctaccagattccttattctcgtccggtaggttcgagactaacagccgcgctttcctcgatgaagccgaggaaaagtttgccgccgctcaccctcatgcctgtttgggagaaattaataagtccaccgtgggaggatcccacttcatcttttcccaatatttaccaccattgctacccgcagacgctgttgccctggtaggtgcttcattggctgggaaagctgctaaagctgcttgcagcgttgttgatgtctatgctccatcatttgaaccttatctacaccctgagacactgagtcgcgtgtacaagattatgatcgatttcaagccgtgtaggcttatggtgtggagaaacgcgaccttttatgtccaagagggtgttgatgcagttacatcagcactagcagctgtgtccaaactcatcaaagtgccggccaatgagcctgtttcattccatgtggcatcagggtacagaaccaacgcgctggtagcgccccaggctaaaatttcaattggagcctacgccgccgagtgggcactgtcaactgaaccgccacctgctggttatgcgatcgtgcggcgatatattgtaaagaggctcctcagctcaacagaagtgttcttgtgccgcaggggtgttgtgtcttccacctcagtgcagaccatttgtgcactagagggatgtaaacctctgttcaacttcttacaaattggttcagtcattgggcccgtgtgactctagagtggacctgttcccatcccccgctcaactactcaggtagtggttcgcggcaacgggtacaccgcagttggtaacaagcttgtcgatggaaaatatggaaaacgacgagaacatcgtggtgggccccaagcccttctaccccatcgaggaaggcagcgccggcacccagctgcggaagtacatggaaagatacgccaagctgggcgccattgccttcaccaacgccgtgaccggcgtggactacagctacgccgagtacctggaaaagagctgctgcctgggcaaggctctgcagaactacggcctggtggtggacggccggatcgccctgtgcagcgagaactgcgaggaattcttcatccccgtgatcgccggcctgttcatcggcgtgggcgtggctcccaccaacgagatctacaccctgcgggagctggtgcacagcctgggcatcagcaagcccaccatcgtgttcagcagcaagaagggcctggacaaagtcatcaccgtgcagaaaaccgtgaccaccatcaagaccatcgtgatcctggacagcaaggtggactaccggggctaccagtgcctggacaccttcatcaagcggaacaccccccctggcttccaggccagcagcttcaagaccgtggaggtggaccggaaagaacaggtggccctgatcatgaacagcagcggcagcaccggcctgcccaagggcgtgcagctgacccacgagaacaccgtgacccggttcagccacgccagggaccccatctacggcaaccaggtgtcccccggcaccgccgtgctgaccgtggtgcccttccaccacggcttcggcatgttcaccaccctgggctacctgatctgcggcttccgggtggtgatgctgaccaagttcgacgaggaaaccttcctgaaaaccctgcaggactacaagtgcacctacgtgattctggtgcccaccctgttcgccatcctgaacaagagcgagctgctgaacaagtacgacctgagcaacctggtggagatcgccagcggcggagcccccctgagcaaagaagtgggagaggccgtcgccaggcggttcaatctgcccggcgtgcggcagggctacggcctgaccgagacaaccagcgccatcatcatcacccccgagggcgacgacaagcctggagccagcggcaaggtggtgcccctgttcaaggccaaagtgatcgacctggacaccaagaagagcctgggccccaacagacggggcgaagtgtgcgtgaagggccccatgctgatgaagggctacgtgaacaaccccgaggccaccaaagagctgatcgacgaagagggctggctgcacaccggcgacatcggctactacgacgaagagaagcacttcttcatcgtggaccggctgaagagcctgatcaagtacaagggctatcaggtgccccctgccgagctggaaagcgtcctgctgcagcaccccagcatcttcgacgccggcgtggccggggtgccagatcctgtggccggcgagctgcctggcgccgtggtggtgctggaatccggcaagaacatgaccgagaaagaagtgatggactacgtcgccagccaggtgtccaacgccaagcggctgagaggcggcgtgagattcgtggacgaagtgccaaagggcctgaccggcaagatcgacggcagggccatccgggagatcctgaagaaacccgtggccaagatgtgattataactcgagggagccatagattcattttgtggtgacgggattttaggtgagtatctagattactttattctgtccgtcccactcttgctgttgcttactaggtatgtagcatctgggttagtgtatgttttgactgccttgttctattcctttgtattagcagcttatatttggtttgttatagttggaagagccttttctactgcttatgcttttgtgcttttggctgcttttctgttattagtaatgaggatgattgtgggtatgatgcctcgtcttcggtccattttcaaccatcgccaactggtggtagctgattttgtggacacacctagtggacctgttcccatcccccgcccaaccactcaggtagtggttcgcggcaacgggtacaccgcagttggtaacaagcttgtcgatggcgtcaagacgatcacgtccgcaggccgcctcttttcgaaacggacggcggcgacagcctacaagctacaatgacctactgcgcatgtttggtcagatgcgggtccgcaaaccgcccgcgcaacccactcaggctattattgcagagcctggagaccttaggcatgatttaaatcaacaggagcgcgccaccctttcgtcgaacgtacaacggttcttcatgattgggcatggttcactcactgcagatgccggaggactcacgtacaccgtcagttgggttcctaccaaacaaatccagcgcaaagttgcgcctccagcagggccgtaagacgtggatattctcctgtgtggcgtcatgttgaagtagttattagccacccaggaaccaaaaaaaaaaaaaaaaaaaaaaaaaaaaaaaaaaaaaaaa

SEQ ID NO: 35 - DNA, 8554 bp, synthetic construct
  misc_feature 1..8554: note = Synthetic polypeptide
  misc_feature 1..8554: note = Construct Alpha-R-eGFP
  source 1..8554: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatgggaagagccggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaggctcttcgtaattaattgatcgatacagcagcaattggcaagctgcttacatagaaggcgcgccgtttaaacggccggccttaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaa

SEQ ID NO: 36 - DNA, 59 bp, synthetic construct
  misc_feature 1..59: note = Synthetic polynucleotide
  misc_feature 1..59: note = 5 human beta globin UTR
  source 1..59: mol_type = other DNA
acatttgcttctgacacaactgtgttcactagcaacctcaaacagacaccgccgccacc

SEQ ID NO: 37 - DNA, 18 bp, synthetic construct
  misc_feature 1..18: note = Synthetic polynucleotide
  misc_feature 1..18: note = T7 Promoter
  source 1..18: mol_type = other DNA
taatacgactcactatag

SEQ ID NO: 38 - DNA, 142 bp, synthetic construct
  misc_feature 1..142: note = Synthetic polynucleotide
  misc_feature 1..142: note = DLP
  source 1..142: mol_type = other DNA
atagtcagcatagtacatttcatctgactaatactacaacaccaccaccatgaatagaggattctttaacatgctcggccgccgccccttcccggcccccactgccatgtggaggccgcggagaaggaggcaggcggccccg

SEQ ID NO: 39 - DNA, 66 bp, synthetic construct; identical to SEQ ID NO: 3
  misc_feature 1..66: note = Synthetic polynucleotide
  misc_feature 1..66: note = P2A
  source 1..66: mol_type = other DNA

SEQ ID NO: 40 - DNA, 846 bp, synthetic construct
  misc_feature 1..846: note = Synthetic polynucleotide
  misc_feature 1..846: note = dsGFP
  source 1..846: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtag

SEQ ID NO: 41 - DNA, 134 bp, synthetic construct
  misc_feature 1..134: note = Synthetic polynucleotide
  misc_feature 1..134: note = 3 Human beta globin UTR
  source 1..134: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 42 - DNA, 29 bp, synthetic construct
  misc_feature 1..29: note = Synthetic polynucleotide
  misc_feature 1..29: note = T7 Terminator
  source 1..29: mol_type = other DNA
aacccctctctaaacggaggggttttttt

SEQ ID NO: 43 - DNA, 1412 bp, synthetic construct
  misc_feature 1..1412: note = Synthetic polynucleotide
  misc_feature 1..1412: note = Sequence of DLP dsGFP Mrna
  source 1..1412: mol_type = other DNA
taatacgactcactatagacatttgcttctgacacaactgtgttcactagcaacctcaaacagacaccgccgccaccatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacccctctctaaacggaggggttttttt

SEQ ID NO: 44 - protein, 9 aa, synthetic construct
  REGION 1..9: note = Synthetic polypeptide
  REGION 1..9: note = misc_feature - H-2 Kd peptide
IYSTVASSL

SEQ ID NO: 45 - protein, 15 aa, synthetic construct
  REGION 1..15: note = Synthetic polypeptide
  REGION 1..15: note = misc_feature - CD4 T cell epitope
KSSFFRNVVWLIKKN

SEQ ID NO: 46 - DNA, 104 bp, synthetic construct
  source 1..104: mol_type = other DNA; note = Sindbis virus DNA
atgaatagaggattctttaacatgctcggccgccgccccttcccggcccccactgccatgtggaggccgcggagaaggaggcaggcggccccgatgcctgcccg

SEQ ID NO: 47 - DNA, 120 bp, synthetic construct
  source 1..120: mol_type = other DNA; note = Aura virus DNA
atgaactctgtcttttacaatccgtttggccgaggtgcctacgctcaacctccaatagcatggaggccaagacgtagggctgcacctgcgcctcgaccatccgggttgactacccagatc

SEQ ID NO: 48 - DNA, 71 bp, synthetic construct
  source 1..71: mol_type = other DNA; note = Eastern Equine Encephalitis virus SA DNA
atgtttccgtatccaacattgaactacccgcctatggcaccggttaatccgatggcatacagggaccccaa

SEQ ID NO: 49 - DNA, 91 bp, synthetic construct
  source 1..91: mol_type = other DNA; note = O'Nyong-Nyong virus DNA
atggagttcataccagcacaaacttactacaatagaagataccagcctagaccctggactcaacgccctactatccaggtgatcaggccaa

SEQ ID NO: 50 - DNA, 67 bp, synthetic construct
  source 1..67: mol_type = other DNA; note = Semliki Forest virus DNA
atgaattacatccctacgcaaacgttttacggccgccggtggcgcccgcgcccggcggcccgtcctt

SEQ ID NO: 51 - DNA, 69 bp, synthetic construct
  source 1..69: mol_type = other DNA; note = Ross River virus DNA
atgaattacataccaacccagactttttacggacgccgttggcggcctcgcccggcgttccgtccatgg

SEQ ID NO: 52 - DNA, 91 bp, synthetic construct
  source 1..91: mol_type = other DNA; note = Mayaro virus DNA
atggatttcctaccaacacaagtgttttatggcaggcgatggagaccacgaatgccgccacgcccttggaggccacgcccacctacaattc